(19) United States
(12) Patent Application Publication
(10) Pub. No.: US 2023/0059390 A1
(43) Pub. Date: Feb. 23, 2023

(54) METHODS AND SYSTEMS FOR MULTIPLEX ALLELE DETECTION

(71) Applicant: GENOMIND, INC., King of Prussia, PA (US)

(72) Inventors: David J. Robbins, New Orleans, LA (US); Kathleen Davis, Havertown, PA (US)

(21) Appl. No.: 17/797,253

(22) PCT Filed: Feb. 4, 2021
(86) PCT No.: PCT/US21/16570

§ 371 Date: Aug. 3, 2022

Related U.S. Application Data
(60) Provisional application No. 62/969,906, filed on Feb. 4, 2020.

(51) Int. Cl. C12Q 1/6883; C12Q 1/686; C12Q 1/6858

(52) U.S. Cl. CPC C12Q 1/6883; C12Q 1/686; C12Q 1/6858; C12Q 2600/106; C12Q 2600/156

(57) ABSTRACT

Methods and systems for determining allele status at a plurality of loci. A first plurality of loci are amplified, in a single reaction vessel, with a plurality of fluorescently labeled detection reagents. A first detection reagent that is specific for the most prevalent allele in a population is labeled with a first fluorescent moiety. A second detection reagent that is specific for a minor allele in the population is labeled with a second fluorescent moiety that is distinguishable from the first fluorescent moiety. A fluorescent signal corresponding to the first fluorescent moiety and the second fluorescent moiety in the reaction vessel is detected. When the contribution of the second fluorescent moiety does not satisfy a threshold, report that the subject does not carry the second allele at any of the first plurality of genomic loci. When the contribution of the second fluorescent satisfies the threshold, perform secondary allele detection assays.

### CROSS-REFERENCE TO RELATED APPLICATIONS

This application claims priority to U.S. Provisional Patent Application No. 62/969,906, filed on Feb. 4, 2020, the disclosure of which is hereby incorporated by reference in its entirety for all purposes.

### TECHNICAL FIELD

The present disclosure relates to the field of pharmacogenetics and, more specifically, to methods and systems for performing multiplexed single nucleotide polymorphism (SNP) detection assays.

### BACKGROUND

Not all patients react to a therapy in a uniform and beneficial manner. A number of factors including age, gender, ethnicity, and environmental and/or behavioral factors can influence the therapeutic efficacy and adverse reactions of therapeutic agents. Importantly, genetic variations among patients have been shown to account for variable drug reactions. Meyer, Urs A., “Pharmacogenetics and adverse drug reactions,” The Lancet 356:1667-71 (2000). For example, citalopram is one of the most commonly prescribed drugs for treating mental illness, such as depression. However, in populations with certain permutations of the gene CYP2C19 (2-4% Caucasians and 8-13% of Asians), administering a normal dosage of citalopram poses a significant risk of drug overexposure and adverse reaction. As such, a dosage adjustment is necessary for these patients. Thus, it is oftentimes beneficial for clinicians to have patients' pertinent genetic profiles available when making decisions, such as prescribing and dosing therapeutic agents.

Over the past 30 years, precision medicine has grown substantially, facilitated particularly by advances in molecular genetics and genotyping technologies. Modern genotyping technology allows for rapid detection and measurement of genetic variations, such as single nucleotide polymorphisms (SNPs), across a large span of the human genome. Over one-hundred million SNPs have been identified in the human population (Auton, A., et al., Nature, 526:68-74 (2015)), making them the most common type of genetic variation in humans. SNPs occur normally throughout the human genome and are mostly clinically insignificant. However, a relatively small portion of SNPs have been identified as important biomarkers associated with susceptibility to certain diseases and/or metabolism of different drugs. Syvanen, A., Nature Genetics, 37:S5-10 (2005). The SNP-based genotyping technology has been reportedly used in a variety of areas such as molecular diagnosis, prenatal analysis, predictive genetic testing, and in particular, pharmacotherapy, giving rise to the concept of “pharmacogenetics.” Roses, A., Nature, 405(3788):857-65 (2000).

Compared to conventional pharmaceutical approaches, where all patients diagnosed with a particular condition are prescribed a common therapy, pharmacogenetics provides personalized treatment based on the genotype profile specific to an individual patient. This allows for more accurate predictions about the patient's susceptibility of developing disease, the progression of the disease, and the patient's likely reaction to treatment. Accordingly, this approach helps clinicians achieve higher drug efficacy, increased drug tolerability, and reduced adverse reactions through better selection of therapeutic agents with dosages optimized for the individual patient.

Because of the rapidly growing understanding of key genetic biomarkers, like SNPs, and the impact the biology underlying the biomarkers has on drug metabolism, many pharmaceuticals have FDA-approved labels that list pertinent genetic biomarkers, warnings particular to specific patient populations, and information about metabolism of the drug relative to such genetic biomarkers and warnings. FDA-approved labels commonly also contain information about pharmacokinetic and pharmacodynamics drug interactions. FDA, “Table of Pharmacogenomic Biomarkers in Drug labeling.” For example, the FDA-approved label for aripiprazole, an atypical antipsychotic drug used to treat schizophrenia and other mental disorders, states that “[d]osage adjustments are recommended in patients who are known CYP2D6 poor metabolizers and in patients taking concomitant CYP3A4 inhibitors or CYP2D6 inhibitors or strong CYP3A4 inducers.” ABILIFY® Prescribing information, Otsuka America Pharmaceutical Inc., 03US19IBR0002, (2019). These genes encode important enzymes that metabolize pharmaceuticals in the liver. As such, best medical practices warrant clinicians to consult with drug labels constantly for gene-drug association information and comply with the label's instructions.

One widely used genotyping technology is the TaqMan® assay. The assay relies on oligonucleotide probes—TaqMan® probes—that recognize and bind complementary DNA sequences with high specificity. Holland, P M et al., PNAS U.S.A. 88(16):7276-80 (1991), the content of which is incorporated herein by reference. A TaqMan® probe is labeled with a donor fluorophore at one end and a corresponding quencher fluorophore at the other end. While the TaqMan® probe is intact, excitation of the donor fluorophore in solution does not result in a detectable fluorescent signal because of the proximity of the quencher fluorophore at the other end of the probe. However, when the probe is degraded by the 5′ to 3′ exonuclease activity of a polymerase, e.g., Taq polymerase, the donor and quencher fluorophores are decoupled and, thus, excitement of the donor fluorophore results in a detectable fluorescent signal because the quencher fluorophore is no longer coupled in nearby proximity. Accordingly, whether or not a particular nucleotide sequence is present in a sample can be detected based on whether or not a donor fluorophore produces a detectable signal when excited.

Specifically, during a TaqMan® assay, a sample containing DNA is subjected to polymerase chain reaction (PCR) in the presence of a first TaqMan® probe complementary to a sequence of interest. Optionally, a second TaqMan® probe that is complementary to a highly similar sequence, e.g., which varies from the sequence targeted by the first TaqMan® probe by a single nucleotide difference, is also included in the assay to compete with the first TaqMan® probe. If the target sequence is not present in the DNA sample, the donor fluorophore on the first TaqMan® probe does not fluoresce when excited because of the presence of the quencher fluorophore at the other end of the probe. However, if the target DNA sequence is present in the sample, the first TaqMan® probe will bind to its target sequence, and then be degraded by the exonuclease activity of the polymerase during the PCR reaction, causing physical decoupling of the donor and quencher fluorophores. As a result, a fluorescence emission from the donor fluorophore, following laser excitation, can be detected. In this way, genotype variations in patient DNAs, such as SNPs, can be detected and analyzed. Shen, G Q et al., “The TaqMan® Method for SNP Genotyping,” In: Komar A. (eds) Single Nucleotide Polymorphisms. Methods in Molecular Biology (Methods and Protocols), vol 578. Humana Press, Totowa, N.J. (2009).

Unfortunately, the clinical adoption/implementation of pharmacogenetic testing by TaqMan® assay is limited by its high cost and low throughput, notwithstanding its capability to improve clinical therapy and reduce healthcare costs for society. This is increasingly true as more gene-drug interactions are identified and larger pharmacogenetic panels are developed. The larger size of these panels increases the amount of biological sample needed from the patient, as well as increases the volume of test reagents needed to run the assays. This is particularly problematic when the genotyping assay requires a whole blood sample from patient, e.g., rather than saliva which is more easily collected. All of these limitations contribute to the time and human expense of administering the assay, making the operation difficult to scale up and meet clinical needs. As a result, conventional genotyping assays fail by not (1) testing multiple patient samples simultaneously and/or (2) analyzing SNPs across a plurality of genomic loci for a single patient simultaneously.

The difficulties associated with in scaling up genotyping assays pose a substantial hurdle to clinicians prescribing therapies with adverse gene-drug interactions, as they often consider multiple pharmaceutical solutions for a single patient. Each of these therapeutic options may be associated with multiple pharmacogenetic interactions, further compounding the problem. For example, when treating patients with similar symptoms of serious mental illness (SMI), clinicians often need to choose an appropriate prescription from a list of more than 30 FDA-approved drugs, many of which have known pharmacogenetic interactions. FDA, “Table of Pharmacogenomic Biomarkers in Drug labeling.” As such, only having a small set of these genes tested can hardly provide clinicians comprehensive and informative evaluation of the patient's drug profile. However, as outlined above, obtaining test results for all known pharmacogenetic interactions can become very expensive.

In addition, the lack of effective means to reduce cost and conduct genotyping assays on a large scale become a more pronounced problem in light of the frequent necessity for analyzing a combination of interrelated SNPs for individual patients. This is partly due to the rapidly increasing number of clinically relevant SNPs and the enhanced understanding of the complex networks among SNPs. In particular, although a single SNP alone may cause a Mendelian disease by disrupting the normal functionality of a single gene, SNPs can operate in coordination with other SNPs at other loci for complex diseases such as cancer, mental disorder, infectious diseases, and autoimmune diseases. Singh M et al., Rheumatology International, 31(3):421-23 (2011). The need for analyzing a combination of interrelated SNPs for individual patients can further increase the total cost of the genotyping assay.

For instance, mental illnesses are highly prevalent in the United States, and a major public health concern impacting nearly one in five adults. Serious mental illness (SMI), defined as a mental, behavioral, or emotional disorder resulting in serious functional impairment which substantially interferes with or limits one or more major life activities, is also prevalent in the U.S. More than 10 million adults, representing 4.2% of the adult U.S. population, have been diagnosed with SMI. NIH, Mental Illness, November 2017. SMI costs more than $193 billion per year in lost earnings in the U.S. Major depressive disorder (MDD), bipolar disorder, schizophrenia, schizoaffective disorder, and other SMIs are associated with increased mortality from various causes, including but not limited to suicide. John A. et al., Schizophr Res., 199:154-62 (2018); Laursen T M et al., J Clin Psychiatry, 68(6):899-907 (2007). In military veterans, posttraumatic stress disorder and traumatic brain injury also increase the risk of suicidal behavior. Wilks C R et al., J Psychiatr Res., 109:139-44 (2019). Because the distinction between serious and any mental illness is not always apparent, even mental illnesses which are not typically thought of as serious may be associated with excess mortality. For example, attention-deficit hyperactivity disorder (ADHD) is associated with excess mortality. In part this may be due to co-morbidities, but this excess remains even when accounting for co-morbid mental health diagnoses. The excess mortality in ADHD is driven mostly by unnatural causes, including accidents. Dalsgaard S. et al., Lancet, 385(9983):2190-96 (2015).

Many individuals suffering with SMI do not respond adequately or completely to initial therapy. For example, among patients with MDD, response to initial treatment fails to occur in approximately half of all individuals; remission is even less frequent. Trivedi M H et al., Am J Psych, 163:28-40 (2006). In MDD, work-related disability and productivity loss are critical determinants of patient quality of life, and contribute significantly to the human and economic costs caused by this disease. Lee et al., J Affect Disord, 227:406-15 (2018). Schizophrenia, another SMI, follows a fairly consistent natural history characterized by initial response to antipsychotic drugs, but subsequent non-adherence, deterioration and recurrent episodes of psychosis. Lieberman J A., J Clin Psychiatry, 67(10):e14 (2006).

In patients with SMI, pharmacogenetic testing has the potential to assist in the selection of drugs which are more likely well tolerated, and to avoid serious adverse events (SAEs), as genetic variation is an important factor that influences the efficacy and tolerability (benefit: risk profile) of pharmaceutical agents, including psychotropic drugs. For example, cytochrome p450 (CYP450) enzymes account for the metabolism of most pharmaceuticals. The identification and validation of these pharmacokinetic genes and of pharmacodynamic gene variants has enabled the emergence of precision medicine in psychiatry. Many pharmaceuticals, including many psychotropic drugs, now have biomarker warnings or precautions in their prescribing information, or contain pertinent information on the agent's metabolism, with respect to the effect of variants of genes encoding for CYP450 enzymes on the drug's exposure. Some product labels also contain information regarding the drug's ability to influence the exposure of concomitantly administered drugs via inhibition or induction of CYP450 enzymes. As the Agency notes, “Pharmacogenomics can play an important role in identifying responders and non-responders to medications, avoiding adverse events, and optimizing drug dose.” FDA, Table of Pharmacogenomic Biomarkers in Drug Labeling.

For example, aripiprazole, a second generation (or atypical) antipsychotic drug, is indicated to treat schizophrenia, mania associated with bipolar disorder, and several other serious disorders. Aripiprazole's label states: “Dosage adjustments are recommended in patients who are known CYP2D6 poor metabolizers and in patients taking concomitant CYP3A4 inhibitors or CYP2D6 inhibitors or strong CYP3A4 inducers.” Aripiprazole's label recommends half the usual starting dose in CYP2D6 poor metabolizers, and the dosage may vary by a factor of 8 in the presence of concomitant inducers or inhibitors of CYP450 enzymes. Abilify® Prescribing Information, 2018. Otsuka America Pharmaceutical Inc.

Inefficacy is an obvious potential consequence of underexposure. Alternatively, excessive exposure may be associated with common and manageable or infrequent and serious tolerability issues, such as orthostasis or tardive dyskinesia. One of the most common drugs used in psychiatry is citalopram. In CYP2C19 poor metabolizers (2-4% of Caucasians and 8-13% of Asians) the AUC exposure to citalopram may be doubled, increasing the risk of QT prolongation. Celexa® (citalopram HBr) Prescribing Information. Forest Laboratories, Inc.

Other biomarkers may also have an important role in the safe use of psychotropics, including the avoidance of SAEs. One example is the presence of the HLA-B*1502 gene variant, which is associated with increased risk for severe and sometimes fatal skin reactions, such as Stevens-Johnson syndrome and toxic epidermal necrolysis, to carbamazepine and oxcarbazepine. Phillips E J et al., Clinical Pharmacogenetics Implementation Consortium Guideline for HLA Genotype and Use of Carbamazepine and Oxcarbazepine: 2017 Update, Clinical Pharmacology & Therapeutics (2018). Carbamazepine is indicated for epilepsy and trigeminal neuralgia, but is widely utilized as a mood stabilizer in bipolar disorder, and as adjunctive therapy in MDD. APA, Practice Guideline for the Treatment of Major Depressive Disorder (3rd ed. 2010). Carbamazepine extended release capsules have the additional indication of acute mania or mixed episodes associated with bipolar I disorder. This risk is highlighted in carbamazepine's current drug label, which contains a boxed warning, and specifically calls for biomarker screening in individuals of Asian descent.

### SUMMARY

Given the background above, there is a need in the art for faster, more cost-effective methods of contemporaneously determining allele status at a plurality of genomic loci. The present disclosure provides solutions to these, as well as other, needs. In part, the advantages of the present disclosure flow from the development of a method for simultaneously screening the allele status of multiple genomic loci, e.g., for one or more subject in a single reaction of a multiplexed assay. In other aspects, the present disclosure provides improved methods for assigning therapy for neuropsychiatric disorders.

The improved multiplexing assays disclosed herein are based on the premise that many of the alleles known to have pharmacogenetic effects are fairly rare in the human population, e.g., having population allele frequencies of less than 5%. Accordingly, it was discovered that genotyping assays for several loci could be performed simultaneously, in the same reaction vessel, as long as the same fluorophore was used to report the presence of the common allele, e.g., the wild type allele, at each loci. Although this schema results in the inability to determine which allele is not wild type, when a variant allele is present at one of the loci being tested, the relative scarcity of the variant alleles in the population renders a majority of the assays confirmatory that all alleles being tested are wild type. Advantageously, this method eliminates the conventional requirement of testing each loci in a separate reaction vessel, thereby significantly increasing the throughput, and reducing the cost, of genotyping assays that query multiple loci.

Accordingly, one aspect of the present disclosure provides a method for determining an allele status at a plurality of genomic loci in a subject. The method includes amplifying, in a single in vitro reaction vessel, a first plurality of genomic loci, by PCR, from nucleic acids isolated from a sample obtained from the subject, in the presence of a plurality of fluorescently-labeled detection reagents. The plurality of fluorescently-labeled detection reagents includes, for each respective genomic locus in the first plurality of genomic loci, a first detection reagent specific for the presence of a first allele at the respective genomic locus, where the first allele is the most prevalent allele in a population of the species of the subject and the first detection reagent is labeled with a first fluorescent moiety, and a second detection reagent specific for the presence of a second allele at the respective genomic locus, where the second allele is a minor allele in the population and the second detection reagent is labeled with a second fluorescent moiety distinguishable from the first fluorescent moiety. The method also includes detecting a fluorescent signal corresponding to the first fluorescent moiety and the second fluorescent moiety in the reaction vessel during or after the amplifying. When the contribution of the second fluorescent moiety to the fluorescent signal detected in the reaction vessel does not satisfy a threshold contribution, the subject is reported to not carry the second allele at any of the first plurality of genomic loci. When the contribution of the second fluorescent moiety to the fluorescent signal detected in the reaction vessel satisfies a threshold contribution, a first plurality of secondary allele detection assays is performed, and the allele status at each of the first plurality of genomic loci is reported based on the first plurality of secondary allele detection assays.

Another aspect of the present disclosure provides a method for performing a high throughput genotyping assay. The method includes dispensing, into each respective well in a first plurality of wells in a multiwell plate, according to one or more template plate definitions associated with the high throughput genotyping assay, a respective template nucleic acid preparation, reagents for amplifying a first plurality of genomic loci, and a first plurality of fluorescently-labeled detection reagents. The respective template nucleic acid preparation dispensed into each respective well is prepared from a respective biological sample obtained from a different test subject in a plurality of test subjects. The first plurality of fluorescently-labeled detection reagents includes, for each respective genomic locus in the first plurality of genomic loci, a first detection reagent specific for the presence of a first allele at the respective genomic locus, where the first allele is the most prevalent allele in a population of the species of the subject and the first detection reagent is labeled with a first fluorescent moiety. The first plurality of fluorescently-labeled detection reagents also includes, for the same respective genomic locus in the first plurality of genomic loci, a second detection reagent specific for the presence of a second allele at the respective genomic locus, where the second allele is a minor allele in the population and the second detection reagent is labeled with a second fluorescent moiety distinguishable from the first fluorescent moiety. The method then includes by amplifying the first plurality of genomic loci in each respective well after the dispensing. The method then includes by detecting in each respective well a fluorescent signal corresponding to the first fluorescent moiety and the second fluorescent moiety in the reaction vessel, during or after the amplifying. When the contribution of the second fluorescent moiety to the fluorescent signal detected in the respective well does not satisfy a threshold contribution, the corresponding subject in the plurality of subjects is reported to not carry the second allele at any of the first plurality of genomic loci. When the contribution of the second fluorescent moiety to the fluorescent signal detected in the respective well satisfies the threshold contribution, a first plurality of secondary allele detection assays using a template nucleic acid preparation from the corresponding subject in the plurality of subjects is performed, where each secondary allele detection assay in the first plurality of secondary allele detection assays determines the allele status at one respective genomic locus in the first plurality of genomic loci, and the allele status of the corresponding subject at each of the first plurality of genomic loci is reported based on the first plurality of secondary allele detection assays.

In another aspect, the present disclosure provides a method for providing guidance for the treatment of a neuropsychiatric disorder in a subject. The method includes determining the allele status for a plurality of genomic loci, where each respective loci in the plurality of loci is associated with a therapeutic efficacy of at least one therapy for a neuropsychiatric disorder. The determining includes amplifying, in a first single in vitro reaction vessel, a first set of two or more genomic loci in the plurality of genomic loci by PCR, from nucleic acids isolated from a sample obtained from the subject, in the presence of a plurality of fluorescently-labeled detection reagents. For each respective genomic locus in the two or more genomic loci, the plurality of fluorescently-labeled detection reagents includes a first detection reagent specific for the presence of a first allele at the respective genomic locus. The first allele is the most prevalent allele in a population of the species of the subject. The first detection reagent is labeled with a first fluorescent moiety. The plurality of fluorescently-labeled detection reagents also includes a second detection reagent specific for the presence of a second allele at the respective genomic locus. The second allele is a minor allele in the population. The second detection reagent is labeled with a second fluorescent moiety distinguishable from the first fluorescent moiety. The method also includes, during or after the amplifying, a fluorescent signal corresponding to the first fluorescent moiety and the second fluorescent moiety in the first single reaction vessel. Responsive to the detecting, when the contribution of the second fluorescent moiety to the fluorescent signal detected in the reaction vessel does not satisfy a threshold contribution, determining that the subject is homozygous for the first allele at each respective loci in the first set of two or more genomic loci. Responsive to the detecting, when the contribution of the second fluorescent moiety to the fluorescent signal detected in the reaction vessel satisfies the threshold contribution, a first plurality of secondary allele detection assays are performed, where each secondary allele detection assay in the first plurality of secondary allele detection assays determines the allele status at one respective genomic locus in the first set of two or more genomic loci, thereby determining the allele status at each respective loci in the first set of two or more genomic loci. The method then includes associating the allele status determined for the plurality of genomic loci with one or more recommendations for the treatment of the neuropsychiatric disorder. The method then includes generating a patient-specific report including the one or more recommendations for the treatment of the neuropsychiatric disorder.

In another aspect, the present disclosure provides a method for providing treatment guidance in a subject. The method includes determining the allele status for a plurality of genomic loci. The determining includes amplifying, in a first single in vitro reaction vessel, a first set of two or more genomic loci in the plurality of genomic loci by PCR, from nucleic acids isolated from a sample obtained from the subject, in the presence of a plurality of fluorescently-labeled detection reagents. For each respective genomic locus in the two or more genomic loci, the plurality of fluorescently-labeled detection reagents includes a first detection reagent specific for the presence of a first allele at the respective genomic locus. The first allele is the most prevalent allele in a population of the species of the subject. The first detection reagent is labeled with a first fluorescent moiety. The plurality of fluorescently-labeled detection reagents also includes a second detection reagent specific for the presence of a second allele at the respective genomic locus. The second allele is a minor allele in the population. The second detection reagent is labeled with a second fluorescent moiety distinguishable from the first fluorescent moiety. During or after the amplifying, the method includes detecting a fluorescent signal corresponding to the first fluorescent moiety and the second fluorescent moiety in the first single reaction vessel. Responsive to the detecting, when the contribution of the second fluorescent moiety to the fluorescent signal detected in the reaction vessel does not satisfy a threshold contribution, determining that the subject is homozygous for the first allele at each respective loci in the first set of two or more genomic loci. Responsive to the detecting, when the contribution of the second fluorescent moiety to the fluorescent signal detected in the reaction vessel satisfies the threshold contribution, the method includes performing a first plurality of secondary allele detection assays, where each secondary allele detection assay in the first plurality of secondary allele detection assays determines the allele status at one respective genomic locus in the first set of two or more genomic loci, thereby determining the allele status at each respective loci in the first set of two or more genomic loci. The method then includes associating the allele status determined for the plurality of genomic loci with one or more recommendations for the treatment of the neuropsychiatric disorder. The method then includes generating a patient-specific report comprising the one or more recommendations for the treatment of a condition within/in fulfillment of an ICD-10 code selected from the group including F31.0, F31.1, F31.2, F31.3, F31.5, F31.6, F31.7, F31.8, F31.9, F32.0, F32.2, F32.3, F32.4, F32.5, F32.8, F32.9, F33.0, F33.1, F33.2, F33.3, F33.4, F33.8, F33.9, F40.0, F40.1, F40.2, F40.8, F40.9, F41.0, F41.1, F41.3, F41.8, F41.9, F42.2, F42.3, F42.4, F42.8, F42.9, F60.5, F90.0, F90.1, F90.2, F90.8, F90.9, F43.1, F84.0, F20.0, F20.1, F20.2, F20.3, F20.5, F20.8, F20.9, F60.0, F60.1, F60.2, F60.3, F60.4, F60.5, F60.6, F60.7, F60.8, F60.9, F07.0, F07.8, F07.9, G89.2, G89.4, F10.1, F10.2, F10.9, F11.1, F11.2, F11.9, F12.1, F12.2, F12.9, F13.1, F13.2, F13.9, F14.1, F14.2, F14.9, F15.1, F15.2, F15.9, F16.1, F16.2, F16.9, F17.2, F18.1, F18.2, F18.9, F19.1, F19.2, F19.9, F55.0, F55.1, F55.2, F55.3, F55.4, and F55.8.

### BRIEF DESCRIPTION OF THE DRAWINGS

FIG. 1 illustrates a block diagram of an example computing device, in accordance with various embodiments of the present disclosure.

FIG. 2A illustrates the results of multiplexed allele discrimination assays for SNPs rs5030863, rs28371685, and rs5030867. The experimental results from 95 DNA patient samples and 1 negative control are shown, in aggregate, as a scatter plot of the intensity of the fluorescent signal emitted by FAM (corresponding to the wild type alleles) as a function of the intensity of the fluorescent signal emitted by VIC (corresponding to the variant alleles).

FIG. 2B illustrates the results of multiplexed allele discrimination assays for SNPs rs17884712, rs72552267, and rs72558187. The experimental results from 95 DNA patient samples and 1 negative control are shown, in aggregate, as a scatter plot of the intensity of the fluorescent signal emitted by FAM (corresponding to the wild type alleles) as a function of the intensity of the fluorescent signal emitted by VIC (corresponding to the variant alleles).

FIG. 3 illustrates the results of multiplexed allele discrimination assays for SNPs rs5030862, and rs56337013. The experimental results from 95 DNA patient samples and 1 negative control are shown, in aggregate, as a scatter plot of the intensity of the fluorescent signal emitted by VIC (corresponding to the wild type alleles) as a function of the intensity of the fluorescent signal emitted by FAM (corresponding to the variant alleles).

### DETAILED DESCRIPTION

### Introduction

In one aspect, the present disclosure provides faster, more cost-effective methods of contemporaneously determining allele status at a plurality of genomic loci. In some embodiments, these improvements are realized by determining the status of multiple alleles, e.g., that are each associated with a pharmacogenetic effect, in a single reaction using a set of detection probes designed such that detection of the most common allele in the human population, at each loci, results in the same type of fluorescent signal. In this fashion, the absence of a fluorescent signal other than that associated with the most common alleles indicates that the subject is homozygous at each loci tested. This efficiency is made possible by the observation that many pharmacogenetic alleles are found infrequently in the human population. Thus, although the presence of a variant allele in such a multiplexed reaction will necessitate occasional retesting of each loci to determine which variant alleles are present, the reactions can be designed such that this occurs with a sufficiently low frequency, resulting in savings in costs and time.

Advantageously, the multiplex genotyping assays described herein increase the capacity of genotyping assays, e.g., through the recognition that multiple rare alleles can be tested for using a single reporting fluorophore. This observation allows a number of genomic loci to be examined in a single genotyping reaction, as compared to conventional methods that test alleles individually. This results in a considerable reduction in the number of reactions that need to be performed. For instance, as outlined in Example 4, combining the detection of two loci in a single genotyping reaction, as done in Example 3, reduces the number of required reactions by half. Likewise, as outlined in Example 4, combining the detection of three loci in a single genotyping reaction, as done in Examples 1 and 2, reduces the number of required reactions by two-thirds. In total, by testing the eight alleles in three multiplexing reactions, as described in Examples 1-3, the efficiency of the testing methodology is improved by 62%. More remarkably, if all eight tests were combined in a single reaction, an 86% improvement in efficiency could be realized. This is significant, as the demand for testing services, and the number of alleles that need to be tested, continue to expand.

In another aspect, the present disclosure provides methods for providing improved guidance for the treatment of a neuropsychiatric disorder. The improvement is realized, at least in part, by the development of a test that determines the allele status for an improved combination of genes. For example, in some embodiments, the tested genes include combinations of the human genes CYP1A2, CYP2B6, CYP2C9, CYP2C19, CYP2D6, CYP3A4/5, SLC6A4, HTR2A, HLA-A, HLA-B, UGT2B15, MTHFR, BDNF, COMT, MC4R, 5HT2C, ADRA2A, ANK3, CACNA1C, DRD2, GRIK1, OPRM1, UGT1A4, and ABCB1. In some embodiments, the improvement is also realized, at least in part, by providing a patient-specific report including recommendations for the treatment of a neuropsychiatric disorder is generated based on the allele status of one or more tested genomic loci for these genes. In some embodiments, the method provided herein is useful for the treatment of a condition in fulfillment of an ICD-10 code.

Advantageously, by providing a comprehensive mental health pharmacogenetic profile, through evaluation of the improved combinations of gene and loci provided herein, clinicians can more intelligently select personalized pharmacotherapy for neuropsychiatric disorders. For example, many pharmaceuticals approved for treating neuropsychiatric disorders have FDA-approved labels that include therapeutic guidance relating to pertinent genetic biomarkers. Information about the allele status of these genetic biomarkers in a patient leads to optimized pharmacotherapy with higher efficacy, lower tolerability and/or lesser adverse drug reactions. Unfortunately, a comprehensive source of this information is not readily available for clinicians, because conventional genotyping assays only test for a limited subset of the genes and alleles necessary to make informed choices when deciding between the many therapeutic options for treatment of neuropsychiatric disorders. The methods of the present disclosure facilitate collection and analysis of this comprehensive data, which improves clinical outcomes for the treatment of neuropsychiatric disorders. Specifically, the methods described herein allow simultaneous screening of all the genes for clinically relevant SNPs. As such, the methods provided herein are capable of providing comprehensive evaluation of available pharmacotherapy options based on the specific genotype of a patient. In addition, the methods provided herein represent a streamlined, nimble process for generating a patient-specific report based on the genotyping assay results that can be readily useful for physicians in a clinical setting.

In various aspects, the present disclosure provides methods for determining the allele status at a plurality of genomic loci in a multiplexed genotyping reaction. These methods are based on detecting the same type of fluorescent signal to indicate the presence of the common allele at each locus, such that detection of a secondary type of fluorescent signal is evidence of the presence of a rare allele at one of the loci being tested. Methodologies for detecting a particular sequence using fluorescence are known in the art. In some embodiments, the methodology for detecting sequences in the multiplexed methods described herein are based on the TaqMan® assay. In this assay, two probes that hybridize to different alleles at the same loci, e.g., which vary only in respect to the difference between the alleles (e.g., a single nucleotide), are labeled with different donor fluorophores on one end of each probe and an acceptor/quencher fluorophore on the other end. When included in a PCR reaction using a polymerase with exonuclease activity, binding of one probe or the other—and thus the presence of one allele or the other—is detected by fluorescence of one of the two fluorophores after the bound probe is degraded by the exonuclease activity of the polymerase which untethers the donor fluorophore from the acceptor/quencher fluorophore.

A detailed description of TaqMan® assays is provided in subsequent sections. However, TaqMan® genotyping assays are well known to a person of ordinary skill in the art. See, for example, Gaedigk et al., Scientific reports, 5:9257 (2015); Shen et al., The TaqMan® method for SNP genotyping, In Single nucleotide polymorphisms, Humana Press, Totowa, N.J., pp. 293-306 (2009); and Schleinitz et al., Targeted SNP genotyping using the TaqMan® assay, In Disease Gene Identification, Humana Press, Totowa, N.J., pp. 77-87 (2011), the disclosures of which are incorporated herein by reference. One of skill in the art will be readily capable of applying the TaqMan® assay to the methods described herein. One of skill in the art will also appreciate that certain changes and modifications may be practiced within the scope of the present disclosure.

PCR techniques are well known in the art, and one of skill in the art is capable of applying PCR to the method described herein, including identifying proper reaction reagents, ascertaining optimal reaction conditions and parameters, and making necessary modifications. Examples of guidelines and protocols for PCR include: Innis et al., (2012), PCR protocols: a guide to methods and applications, Academic press; and Logan et al., (2009), Real-time PCR: current technology and applications, Horizon Scientific Press, the contents of which are expressly incorporated herein by reference, in their entireties, for all purposes. In some embodiments, where the allele status of a plurality of loci are being detected in a single reaction, a multiplex PCR reaction is used. In other embodiments, where the allele status of a single locus is being detected, a standard PCR reaction is used. In one embodiment, the reagents for the amplifying include a TaqMan® Genotyping Master Mix.

As described herein, the template nucleic acids for the detection assay are obtained from a biological sample from the subject being tested. Techniques for extracting nucleic acids from a sample of a test subject are well known in the art. Commercial kits and protocols for nucleic acid extraction are also widely available. In one embodiment, nucleic acids are isolated from biological samples obtained from buccal cells of a subject. In another embodiment, nucleic acids are isolated from biological samples obtained from saliva of a subject. In yet another embodiment, nucleic acids are isolated from biological samples obtained from saliva of a subject blood of a test subject. In some embodiments, the template nucleic acids isolated from a sample obtained from a subject are DNA molecules, e.g., genomic DNA. In some embodiments, the template nucleic acids isolated from a sample obtained from a subject are RNA molecules, e.g., mRNA transcripts. In one embodiment, the test subject is a human. In another embodiment, the test subject is a mammal.

### Definitions

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

As used herein, the term “biological sample,” “patient sample,” or “sample” refers to any sample taken from a subject, which includes nucleic acids reflecting the genotype of the subject with respect to the loci described herein. Examples of biological samples include, but are not limited to, blood samples, saliva samples, buccal cell samples, and the like. The term “nucleic acid” refers to deoxyribonucleic acid (DNA) and ribonucleic acid (RNA). The nucleic acid in the sample can be a cell-free nucleic acid. A sample can be a liquid sample or a solid sample (e.g., a cell or tissue sample).

As used herein, the term “genomic locus” or “locus” refers to a position (e.g., a site) within a genome, i.e., on a particular chromosome. In some embodiments, a locus refers to a single nucleotide position within a genome, i.e., on a particular chromosome. In some embodiments, a locus refers to a small group of nucleotide positions within a genome, e.g., as defined by a mutation (e.g., substitution, insertion, or deletion) of consecutive nucleotides within a cancer genome. Because normal mammalian cells have diploid genomes, a normal mammalian genome (e.g., a human genome) will generally have two copies of every locus in the genome, or at least two copies of every locus located on the autosomal chromosomes, i.e., one copy on the maternal autosomal chromosome and one copy on the paternal autosomal chromosome.

As used herein, the term “allele” refers to a particular sequence of one or more nucleotides at a genomic locus. Because normal mammalian cells have diploid genomes, a normal mammalian genome (e.g., a human genome) will have two alleles for each genomic locus, which may be the same or different. When a mammal has the same allele at both copies of a locus, they are homozygous for the allele. When an organism has different alleles at their two copies of a locus, they are heterozygous for the two alleles. Accordingly, in some embodiments, the “allele status” or “allelic status” of a mammal at a genomic locus may be homozygous for an allele (e.g., the wild type or most prevalent allele) or heterozygous (e.g., having one copy of the wild type or most prevalent allele and one copy of a variant allele or less prevalent allele). In some embodiments, determining the allelic status of a locus of a mammal includes determining whether the mammal carries, e.g., has at least one copy of, an allele with a known pharmacogenetic effect. In some embodiments, when it is determined that the mammal carries at least one copy of the particular allele, it is determined whether the mammal is homozygous or heterozygous for the particular allele. For example, in some embodiments, this is done when the pharmacogenetic effect of the particular allele is known to be dosage-dependent, that is, when the pharmacogenetic effect of the allele is different when the subject is homozygous for the particular allele than when the subject is heterozygous for the allele. However, in other embodiments, determining the allelic status of a locus of a subject only includes determination of whether the subject carries the particular allele, regardless of the copy number of the particular allele.

As used herein, the term “SNP” or “single nucleotide polymorphism” refers to refers to a substitution of one nucleotide to a different nucleotide at a position (e.g., locus) of a nucleotide sequence, e.g., of a chromosome or genome. A substitution from a first nucleotide X to a second nucleotide Y may be denoted as “X>Y.” For example, a cytosine to thymine SNP may be denoted as “C>T.” Because a SNP corresponds to a nucleotide substitution at a particular position of a genome, e.g., a particular position on a particular chromosome and/or particular gene, a SNP may be used to identify a particular locus. For instance, rs5030863 is a G>C SNP in the CYP2D6 gene, located at position 42525912 of chromosome 22 of human reference genome build GRCh37 37.1/131. Accordingly, the allele corresponding to the rs5030863 SNP is encompassed by the CYP2D6 gene at position 42525912 of chromosome 22 of human reference genome build GRCh37 37.1/131. As such, when a locus corresponding to a particular SNP is amplified, the nucleotide position of the SNP, as well as an appropriate number of nucleotides flanking the SNP, are amplified. Generally, this involves amplifying a region of the genome that is smaller than the entire gene in which the SNP is located. In some embodiments, primers are designed to amplify a region of at least 20 nt, or at least 25 nt, at least 30 nt, at least 40 nt, at least 50 nt, at least 75 nt, at least 100 nt, at least 150 nt, at least 200 nt, at least 250 nt, or more nucleotides encompassing the particular SNP.

As used herein, the term “template plate definition” refers to a plan defining which reagents will be included at which concentrations for a plurality of reactions performed in a plurality of wells of a multiwell plate. In some embodiments, where multiple reactions are prepared simultaneously, e.g., using an automated liquid handler, the template plate definition is defined in electronic instructions (e.g., software), which directs machinery (e.g., the automated liquid handler) to prepare the reactions. In other embodiments, the template plate definition is not defined in electronic instructions, e.g., when the reactions are prepared manually.

As used herein, the term “fluorescent moiety” refers to one or more fluorescent entities whose total fluorescence is such that the moiety may be detected. Thus, a fluorescent moiety may comprise a single entity (e.g., a Quantum Dot or fluorescent molecule) or a plurality of entities (e.g., a plurality of fluorescent molecules). Non-limiting example of fluorescent moieties include 4-acetamido-4′-isothiocyanatostilbene-2,2′-disulfonic acid, acridine, acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate, N-(4-anilino-1-naphthyl)maleimide; anthranilamide, BODIPY, Brilliant Yellow, coumarin, 7-amino methylcoumarin (AMC, Coumarin 120), 7-amino-4-trifluoromethylcouluarin (Coumaran 151), cyanine dyes, cyanosine, 4′,6-diaminidino-2-phenylindole (DAPI), 5′,5″-dibromopyrogallol-sulfonaphthalein (Bromopyrogallol Red), 7-diethylamino-3-(4′-isothiocyanatophenyl)-4-methylcoumarin, diethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylaminolnaphthalene-1-sulfonyl chloride (DNS, dansylchloride), 4-dimethylaminophenylazophenyl-4′-isothiocyanate (DABITC), eosin, eosin isothiocyanate, erythrosin, erythrosin B, isothiocyanate, ethidium, fluorescein, 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC), fluorescamine, IR144, IR1446, Malachite Green isothiocyanate, 4-methylumbelliferoneortho cresolphthalein, nitrotyrosine, pararosaniline, Phenol Red, B-phycoerythrin, o-phthaldialdehyde, pyrene and derivatives: pyrene, pyrene butyrate, succinimidyl 1-pyrene, butyrate quantum dots, Reactive Red 4 (Cibacron™ Brilliant Red 3B-A) rhodamine, 6-carboxy-X-rhodamine (ROX), 6-carboxyrhodamine (R6G), lissamine rhodamine B sulfonyl chloride rhodamine (Rhod), R110, rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, rosolic acid, Cy 3, Cy 5, Cy 5.5, Cy 7, IRD 700, IRD 800, La Jolla Blue, phthalo cyanine, Oregon green, and naphthalo cyanine.

As described herein, the assays used to determine allelic status incorporate two, distinguishable fluorescent moieties. That is, the assays use a first fluorescent moiety, which contributes a first fluorescent signal at a first wavelength, to indicate presence of a first allele and a second fluorescent moiety, which contributes a second fluorescent signal at a second wavelength, to indicate presence of a second allele. Accordingly, when assaying for the presence of a plurality of non-prevalent alleles in a multiplexed assay, as described herein, the contribution of the fluorescent signal corresponding to any particular non-prevalent allele is expected to be only a fraction of the overall fluorescent signal generated in the assay. For example, six different alleles are detected in a multiplex assay targeting three loci, i.e., two alleles at each of the three loci. As such, assuming that all the alleles are equally represented in the sample and that the fluorescent molecules contribute equally to the overall fluorescent signal, any one particular allele contributes approximately one sixth of the overall fluorescent signal generated by the assay. The threshold contribution is a level of fluorescence, detected from fluorophores that correspond to variant alleles, that indicates the presence of one or more variant alleles at a loci being tested. As such, the skilled artisan will know how to select a “threshold contribution,” indicating the presence of a non-prevalent allele, accounting for variables such as variations in the expected relative amount of each particular allele in the sample, relative fluorescent efficiencies, relative detector sensitivities at different wavelengths, and indiscriminate probe binding. For instance, assuming that each particular allele contributes equally to the overall fluorescent signal of a multiplex assay targeting three alleles, the skilled artisan might select a threshold contribution at or slightly below one sixth of the total signal, or one seventh, one eighth, one ninth, or less of the total signal. In some embodiments, where the absence of a most prevalent allele (as opposed to the presence of any particular less prevalent allele) is being assayed for, it might be expected that a less prevalent allele that does not correspond to a probe would be bound approximately evenly by a probe directed to the most prevalent allele and a probe directed to a different less prevalent allele and, therefore, only generate half of the variant allele signal expected to be generated by the less prevalent allele targeted by the probe.

U.S. Patent Application Publication No. 2001/0018184 discloses labeling of a nucleotide probe with a corresponding pair of donor and quencher fluorophores, e.g., in which a donor fluorophore is attached to the γ-phosphate of the polyphosphate moiety, and a quencher is linked elsewhere on the probe, preferably linked to the 5′ carbon of pyrimidine bases and to the 7′ carbon of deazapurine bases.

### I. Multiplex Genotyping Assay

In some embodiments, the methods described herein include steps of (1) amplifying a plurality of genomic loci in a sample in the presence of fluorescently-labeled probes, (2) detecting fluorescent signal from the probes corresponding to the particular alleles present in the sample, and (3) either reporting the status of the alleles when no variant alleles are present or performing secondary allele detection assays when at least one less prevalent allele is present in the sample.

A. Multiplex Assay Containing a First Plurality of Genomic Loci

In some embodiments, a method is provided for determining an allele status at a plurality of genomic loci in a subject. The method includes amplifying, in a single in vitro reaction vessel, a first plurality of genomic loci, by PCR, from nucleic acids isolated from a sample obtained from the subject, in the presence of a plurality of fluorescently-labeled detection reagents. The plurality of fluorescently-labeled detection reagents includes, for each respective genomic locus in the first plurality of genomic loci, a first detection reagent specific for the presence of a first allele at the respective genomic locus, where the first allele is the most prevalent allele in a population of the species of the subject and the first detection reagent is labeled with a first fluorescent moiety, and a second detection reagent specific for the presence of a second allele at the respective genomic locus, where the second allele is a minor allele in the population and the second detection reagent is labeled with a second fluorescent moiety distinguishable from the first fluorescent moiety. The method also includes detecting a fluorescent signal corresponding to the first fluorescent moiety and the second fluorescent moiety in the reaction vessel during or after the amplifying. When the contribution of the second fluorescent moiety to the fluorescent signal detected in the reaction vessel does not satisfy a threshold contribution, (e.g., when contribution of the second fluorescent moiety to the fluorescent signal detected is less than the threshold contribution) the subject is reported to not carry the second allele at any of the first plurality of genomic loci. When the contribution of the second fluorescent moiety to the fluorescent signal detected in the reaction vessel satisfies a threshold contribution, a first plurality of secondary allele detection assays is performed, and the allele status at each of the first plurality of genomic loci is reported based on the first plurality of secondary allele detection assays.

1. Amplifying Target Loci

In some embodiments, the methods described herein include amplifying, in a single in vitro reaction vessel, a first plurality of genomic loci, by polymerase chain reaction (PCR), from nucleic acids isolated from a sample obtained from a subject, in the presence of a plurality of fluorescently-labeled detection reagents. The plurality of fluorescently-labeled detection reagents includes, for each respective genomic locus in the first plurality of genomic loci, a first detection reagent that is specific for the presence of a first allele at the respective genomic locus, where the first allele is the most prevalent allele in a population of the species of the subject and the first detection reagent is labeled with a first fluorescent moiety, and a second detection reagent that is specific for the presence of a second allele at the respective genomic locus, where the second allele is a minor allele in the population and the second detection reagent is labeled with a second fluorescent moiety that is distinguishable from the first fluorescent moiety.

In some embodiments, amplifying a plurality of genomic loci comprises performing a polymerase chain reaction (PCR) on a sample of nucleic acid that comprises the plurality of genomic loci, wherein the copy number of the nucleic acid increases in the reaction. PCR techniques that can be used include but are not limited to digital PCR (dPCR), quantitative PCR (qPCR) or real-time PCR (e.g., TaqMan PCR; Applied Biosystems), reverse-transcription PCR (RT-PCR), allele-specific PCR, amplified fragment length polymorphism PCR (AFLP PCR), colony PCR, Hot Start PCR, in situ PCR (ISH PCR), inverse PCR (IPCR), long PCR, multiplex PCR, or nested PCR.

In some embodiments, the single in vitro reaction vessel is a reaction tube. In some embodiments, the single in vitro reaction vessel is a PCR reaction tube. In some embodiments, the single in vitro reaction vessel is an Eppendorf tube. In other embodiments, the single in vitro reaction vessel is a reaction well in a multiwell plate.

In some embodiments, the plurality of genomic loci include at least two genomic loci. In some embodiments, the plurality of genomic loci include at least three genomic loci. In some embodiments, the plurality of genomic loci include at least four, five, six, seven, eight, nine, ten, or more genomic loci.

In some embodiments, the genomic loci are associated with a particular condition or a class of related conditions, e.g., neuropsychiatric disorders, diabetes, cardiovascular disease (e.g., heart disease), hypertension, Alzheimer's disease, cancer, obesity, arthritis, or asthma. In one embodiment, the genomic loci are associated with alleles known to have pharmacogenetic effects, such that the results of the assay inform treatment of the underlying condition. For instance, in some embodiments, the genomic loci are associated with neuropsychiatric disorders, for instance with alleles at which SNPs having pharmacogenetic effects on various therapies used to treat neuropsychiatric disorders have been identified. Non-limiting examples of such SNPs having pharmacogenetic effects on various therapies used to treat neuropsychiatric disorders, and the loci they are associated with, are provided in Table 1 and Table 2, below. In some embodiments, the genomic loci amplified in the methods described herein are selected from those loci shown in Table 1 and/or Table 2. In some embodiments, the genomic loci include at least four, five, six, seven, eight, nine, ten, or more genomic loci listed in Table 1 and/or Table 2. Further description of the pharmacogenetic effects, and the therapy recommendations derived therefrom, are provided in the section titled “Single nucleotide polymorphism (SNP) markers,” below.

In one embodiment, the first plurality of genomic loci include the human alleles corresponding to the SNPs rs5030863, rs28371685, and rs5030867. Specifically, rs5030863 is a SNP at position 42525912 of human chromosome 22, in the CYP2D6 gene. The presence of SNP rs5030863 is associated with inactivity of P450 2D6. Accordingly, in some embodiments, the methods provided herein further include administering, to the subject, a low dose of a pharmaceutical agent that is metabolized by cytochrome P450 2D6 (CYP2D6) when the subject is determined to carry the rs5030863 SNP. rs28371685 is a SNP at position 94981224 of human chromosome 10, in the CYP2C9 gene. The presence of SNP rs28371685 is associated with reduced activity of P450 2C9. Accordingly, in some embodiments, the methods provided herein further include administering, to the subject, a low dose of a pharmaceutical agent that is metabolized by cytochrome P450 2C9 (CYP2C9) when the subject is determined to carry the rs28371685 SNP. rs5030867 is a SNP at position 42127856 of human chromosome 22, in the CYP2D6 gene. The presence of SNP rs5030867 is associated with inactivity of P450 2D6. Accordingly, in some embodiments, the methods provided herein further include administering, to the subject, a low dose of an antipsychotic drug when the subject is determined to carry the rs5030867 SNP.

In one embodiment, the first plurality of genomic loci include the human alleles corresponding to the SNPs rs17884712, rs72552267, and rs72558187. Specifically, rs17884712 is a SNP at position 94775489 of human chromosome 10, in the CYP2C19 gene. The presence of SNP rs17884712 is associated with reduced activity of P450 2C19. Accordingly, in some embodiments, the methods provided herein further include administering, to the subject, a low dose of a pharmaceutical agent that is metabolized by cytochrome P450 2C19 (CYP2C19) when the subject is determined to carry the rs17884712 SNP. rs72552267 is a SNP at position 94775453 of human chromosome 10, in the CYP2C19 gene. The presence of SNP rs72552267 is associated with reduced activity of P450 2C19. Accordingly, in some embodiments, the methods provided herein further include administering, to the subject, a low dose of a pharmaceutical agent that is metabolized by cytochrome P450 2C19 (CYP2C19) when the subject is determined to carry the rs72552267 SNP. rs72558187 is a SNP at position 94941958 of human chromosome 10, in the CYP2C9 gene. The presence of SNP rs72558187 is associated with reduced activity of P450 2C9. Accordingly, in some embodiments, the methods provided herein further include administering, to the subject, a low dose of a pharmaceutical agent that is metabolized by cytochrome P450 2C9 (CYP2C9) when the subject is determined to carry the rs72558187 SNP.

In one embodiment, the first plurality of genomic loci include the human alleles corresponding to the SNPs rs5030862 and rs56337013. Specifically, rs5030862 is a SNP at position 42130668 of human chromosome 22, in the CYP2D6 gene. The presence of SNP rs5030862 is associated with inactivity of P450 2D6. Accordingly, in some embodiments, the methods provided herein further include administering, to the subject, a low dose of a pharmaceutical agent that is metabolized by cytochrome P450 2D6 (CYP2D6) when the subject is determined to carry the rs5030862 SNP. rs56337013 is a SNP at position 94852738 of human chromosome 10, in the CYP2C19 gene. The presence of SNP rs56337013 is associated with reduced activity of P450 2C19. Accordingly, in some embodiments, the methods provided herein further include administering, to the subject, a low dose of a pharmaceutical agent that is metabolized by cytochrome P450 2C19 (CYP2C19) when the subject is determined to carry the rs56337013 SNP.

Generally, in order for the multiplex reaction to reduce the total number of detection reactions needed, the wild type allele must be prevalent enough in the population such that identification of a variant allele will be a fairly rare occurrence. Accordingly, in some embodiments, for each respective genomic locus in the first plurality of genomic loci, the frequency of the first allele in the population is at least 80%. In some embodiments, for each respective genomic locus in the first plurality of genomic loci, the frequency of the first allele in the population is at least 85%. In some embodiments, for each respective genomic locus in the first plurality of genomic loci, the frequency of the first allele in the population is at least 90%. In some embodiments, for each respective genomic locus in the first plurality of genomic loci, the frequency of the first allele in the population is at least 95%. In some embodiments, for each respective genomic locus in the first plurality of genomic loci, the frequency of the first allele in the population is at least 96%, 97%, 98%, or 99%. Similarly, in some embodiments, for each respective genomic locus in the first plurality of genomic loci, the frequency of the second allele in the population is no more than 20%, or no more than 15%, 10%, 5%, 4%, 3%, 2%, or 1%. Accordingly, in some embodiments, the combined frequency, in the population, of the second allele for each respective loci in the first plurality of loci is no more than 40%. In some embodiments, the combined frequency, in the population, of the second allele for each respective loci in the first plurality of loci is no more than 30%. In some embodiments, the combined frequency, in the population, of the second allele for each respective loci in the first plurality of loci is no more than 20%. In some embodiments, the combined frequency, in the population, of the second allele for each respective loci in the first plurality of loci is no more than 10%. In some embodiments, the combined frequency, in the population, of the second allele for each respective loci in the first plurality of loci is no more than 5%, 4%, 3%, 2%, or 1%.

Accordingly, in some embodiments, for each respective genomic locus in the first plurality of genomic loci, the first detection reagent comprises a first oligonucleotide labeled with a first matching pair of electronic energy transfer chromophores, wherein the sequence of the first oligonucleotide is complementary to the first allele at the respective genomic locus. For each respective genomic locus in the first plurality of genomic loci, the second detection reagent comprises a second oligonucleotide labeled with a second matching pair of electronic energy transfer chromophores, wherein the sequence of the second oligonucleotide is complementary to the second allele at the respective genomic locus.

In some embodiments, the emission spectra of fluorescent signal corresponding to the first and the second excitation chromophores are distinguishable enough in wavelength so as to allow independent detection for each excitation chromophore. In other embodiments, the emission spectra of the first and the second excitation chromophores overlap to the extent that the detection of fluorescence signal from both chromophores can be combined into one single detection. In a specific embodiment, the wavelength centers of emission spectra of the first and the second excitation chromophores are within 20 nm or less, or within 15 nm, 10 nm, 5 nm, or less.

In some embodiments, for each respective genomic locus in the first plurality of genomic loci, the first matching pair of electronic energy transfer chromophores consists of a first excitation chromophore and a first quenching chromophore for the first excitation chromophore; and the second matching pair of electronic energy transfer chromophores consists of a second excitation chromophore and a second quenching chromophore for the second excitation chromophore. Electronic energy transfer chromophores, as well as excitation and quenching effect, are commonly known to a person of ordinary skill in the art, and their description is provided in subsequent sections.

In one specific embodiment, for each respective genomic locus in the first plurality of genomic loci: one of the first matching pair of electronic energy transfer chromophores or the second matching pair of electronic energy transfer chromophores consists of a 6-carboxyfluorescein excitation chromophore and a 5-carboxytetramethylrhodamine quenching chromophore. The other of the first matching pair of electronic energy transfer chromophores or the second matching pair of electronic energy transfer chromophores consists of a 2′-chloro-7′phenyl-1,4-dichloro-6-carboxy-fluorescein excitation chromophore and a 5-carboxytetramethylrhodamine quenching chromophore. The selection of matching pair of EET chromophores is described in detail in subsequent sections.

In some embodiments, the plurality of fluorescently-labeled detection reagents are configured for a TaqMan® assay. These fluorescently-labeled detection reagents (probes) for TaqMan® assays are well-known in the art. A detailed description of the probes are provided in subsequent sections. However, generally, these probes include a polynucleotide with a sequence that is specific for a first allele at the locus of interest having a fluorescent moiety and a quencher moiety attached to the polynucleotide at a distance apart that is sufficient to allow for quenching of the fluorescent moiety by the quencher.

It was found that some combinations of probes result in a shift of the signal away from linearity when used in a TaqMan® assay. This is likely caused by non-specific binding. For instance, in some embodiments, probes directed to the same locus differ by only a single nucleotide. This results in less than 100% specificity. Since all of the reactions in a multiplex assay are run under a common set of conditions (temperature, salt, etc.), the conditions of any given reaction may not be optimal for all sets of probes. Further, in some cases, reagents for two loci may interfere with each other. This could be due to factors such as proximity of the loci or complementary reagent DNA sequences that could cause erroneous signal generation or suppression. Accordingly, in some embodiments, a putative set of loci/probe pairs for a multiplex assay should be tested, to determine empirically whether the particular combination of loci/probes are appropriate for a multiplexing reaction.

2. Detecting Fluorescent Signal

The methods then include detecting fluorescent signal corresponding to the first fluorescent moiety and the second fluorescent moiety in the reaction vessel.

In some embodiments, the detecting occurs during the amplifying. For instance, in some embodiments, the reaction is performed using a quantitative PCR thermal cycler. In some embodiments, the quantitative PCR thermal cycler uses an LED light source to excite the fluorescent moieties at excitation wavelengths, and then measures the emission at corresponding emission wavelengths. In some embodiments, e.g., where a TaqMan® reaction is employed, the detection of an emission wavelength indicates that the fluorescent moiety has been physically separated from the quencher moiety, evidencing the presence of the target allele in the assay sample.

In some embodiments, the detecting occurs after the amplifying. For instance, in some embodiments, the amplifying is performed in a thermal cycler, which may or may not be a quantitative PCR thermal cycler, and the product is evaluated for presence of the probe fluorophores, e.g., where detection of the emission wavelength indicates the presence of the target allele in the assay sample.

3. Reporting Allele Status

The methods then include, responsive to the detection, (i) when the contribution of the second fluorescent moiety to the fluorescent signal detected in the reaction vessel does not satisfy a threshold contribution, reporting that the subject does not carry the second allele at any of the first plurality of genomic loci, and (ii) when the contribution of the second fluorescent moiety to the fluorescent signal detected in the reaction vessel satisfies the threshold contribution, (e.g., when contribution of the second fluorescent moiety to the fluorescent signal detected is greater than the threshold contribution) performing a first plurality of secondary allele detection assays, where each secondary allele detection assay in the first plurality of secondary allele detection assays determines the allele status at one respective genomic locus in the first plurality of genomic loci, and reporting the allele status at each of the first plurality of genomic loci based on the first plurality of secondary allele detection assays.

The threshold contribution is a level of fluorescence, detected from fluorophores that correspond to variant alleles, that indicates the presence of one or more variant alleles at a loci being tested. Accordingly, satisfaction of a threshold contribution (e.g., by detecting a contribution of the second fluorescent moiety to the fluorescent signal that is greater than the threshold contribution) indicates the presence of at least one non-prevalent allele (minor allele) in one or more genomic loci tested. As described above, the threshold level is set based on considerations such as, the number of alleles being tested, the relative efficiencies of all fluorophores used in the reaction, the relative sensitivity of the detectors used for the fluorophores, the expected ratio of alleles represented in the sample, whether the presence of alternate alleles is desired, etc. Although each probe for a particular locus is specific for one allele or the other, the identity in the probe region flanking the SNP results in some cross-hybridization, such that some baseline signal from the second fluorophore is expected even when the sample does not contain a variant allele. Accordingly, the threshold contribution should be set to account for this baseline signal.

Accordingly, in some embodiments, the threshold contribution is set such that it is satisfied when the sample includes nucleic acids from a subject carrying a variant allele in at least one locus being tested. For instance, the contribution of the second fluorescent moiety to the fluorescent signal detected in the reaction vessel satisfies the threshold contribution when the contribution of the second fluorescent moiety to the fluorescent signal indicates that, for at least one respective genomic locus in the plurality of genomic loci, the first allele is not present in at least one half of the nucleic acids encompassing the respective loci. For example, where the reaction includes nucleic acids from a single subject, and three different loci are being tested, the threshold contribution is set such that it is satisfied when at least one sixth of the total fluorescent signal, accounting for variables as discussed above, is attributable to the second fluorophore.

Similarly, in some embodiments, the contribution of the second fluorescent moiety to the fluorescent signal detected in the reaction vessel does not satisfy the threshold contribution when the contribution of the second fluorescent moiety to the fluorescent signal indicates that, for each respective genomic locus in the plurality of genomic loci, the first allele is present in more than one half of the nucleic acids encompassing the respective loci. That is, the threshold contribution is satisfied when the subject does not carry a variant allele at any of the loci being tested.

In some embodiments, when at least one of the subject's two copies of a respective genomic locus carries a third allele, i.e., an allele other than the first allele or the second allele, the first probe and the second probe will bind to the nucleic acids encompassing the third allele with similar affinity, such that approximately a quarter of the fluorescent signal attributable to the loci will be from the second detection probe, assuming that the other copy of the loci contains the first (most prevalent) allele. Accordingly, in some embodiments, e.g., when it is desirable to detect the presence of a third allele at a loci, the threshold contribution is set such that it is satisfied when at least one quarter of the fluorescent signal from any locus being tested is attributable to the second fluorophore. For example, where the reaction includes nucleic acids from a single subject, and three different loci are being tested, the threshold contribution is set such that it is satisfied when at least one twelfth of the total fluorescent signal, accounting for variables as discussed above, is attributable to the second fluorophore.

In some embodiments, the threshold contribution of the second fluorescent moiety is determined by referring to the contribution of the second fluorescent moiety to the fluorescent signal corresponding to both a positive control subject and a negative control subject.

When the contribution of the second fluorescent moiety to the fluorescent signal detected in the reaction vessel satisfies the threshold contribution, the allele status of each genomic locus is then determined individually. This is because the multiplex assay is unable to determine which of the loci carries the variant allele, since each variant allele is associated with the same fluorophore (i.e., the second fluorophore). These secondary allele detection assays can be performed according to any known method for determining the identity of an allele, including sequencing, qPCR, hybridization, and TaqMan® assay. In some embodiments, the secondary allele detection assays are performed using non-multiplexed TaqMan® assays.

In some embodiments, when it is determined that a subject carries a minor allele at a respective genomic locus in the plurality of genomic loci that is associated with a pharmacogenetic effect, a warning, precaution, or drug interaction for a pharmaceutical agent associated with the minor allele of the respective loci is reported, e.g., to a healthcare professional and/or directly to the subject.

B. Multiplex Assay Containing a Second Plurality of Genomic Loci

In some embodiments, a second plurality of loci can be tested in the same reaction vessel, i.e., in the same reaction, as the first plurality of loci, by using a different set of fluorescent moieties for the prevalent and variant alleles that is distinguishable from the fluorescent moieties used to detect the alleles in the first plurality of loci. In such fashion, an even larger number of loci can be tested in a single reaction.

Accordingly, in some embodiments of the amplifying step described above, a second plurality of genomic loci is amplified, by PCR, from the nucleic acids isolated from the sample obtained from the subject, in the presence of the plurality of fluorescently-labeled detection reagents. For each respective genomic locus in the second plurality of genomic loci, the plurality of fluorescently-labeled detection reagents includes (i) a first detection reagent that is specific for the presence of a first allele at the respective genomic locus, where the first allele is the most prevalent allele in a population of the species of the subject and the first detection reagent is labeled with a third fluorescent moiety that is distinguishable from the first fluorescent moiety and the second fluorescent moiety, and (ii) a second detection reagent that is specific for the presence of a second allele at the respective genomic locus, where the second allele is a minor allele in the population and the second detection reagent is labeled with a fourth fluorescent moiety that is distinguishable from the first fluorescent moiety, the second fluorescent moiety, and the third fluorescent moiety. Generally, the emission spectra of fluorescent signals corresponding to the third and the fourth fluorescent moieties are distinguishable enough in wavelength so as to allow independent detection from each other, as well as from the first and second fluorescent moieties.

Similarly, in some embodiments of the detecting step described above, fluorescent signal corresponding to the third fluorescent moiety and the fourth fluorescent moiety are also detected in the reaction vessel, e.g., using separate detection channels that those used to detect fluorescent signal corresponding to the first and second fluorescent moieties.

Likewise, in some embodiments of the reporting step described above, responsive to the detecting, (i) when the contribution of the fourth fluorescent moiety to the fluorescent signal detected in the reaction vessel does not satisfy a threshold contribution, the method includes reporting that the subject does not carry the second allele at any of the second plurality of genomic loci, and (ii) when the contribution of the fourth fluorescent moiety to the fluorescent signal detected in the reaction vessel satisfies the threshold contribution, the method includes performing a second plurality of secondary allele detection assays, wherein each secondary allele detection assay in the second plurality of secondary allele detection assays determines the allele status at one respective genomic locus in the second plurality of genomic loci, and reporting the allele status at each of the second plurality of genomic loci based on the second plurality of secondary allele detection assays.

Likewise, in some embodiments, even more pluralities of genomic loci can be tested in a single multiplex reaction, as long as the corresponding wild type and variant probes employ fluorophores that are distinguishable from all other fluorophores used in the reaction. That is, in order to test a third plurality of loci, probes having a fifth fluorescent moiety that is distinguishable from the first, second, third, and fourth fluorescent moieties could be used to detect the most prevalent allele at each locus in the third plurality of loci, and probes having a sixth fluorescent moiety that is distinguishable from the first, second, third, fourth, and fifth fluorescent moieties could be used to detect the variant allele at each locus in the third plurality of loci. A similar approach could be used to assay for a fourth, fifth, sixth, etc., plurality of loci, limited only by the availability of distinguishable fluorophores and devices capable of differentiating and detecting each of the fluorophores.

### II. High-Throughput Multiplex Genotyping Assay

One aspect of the present disclosure provides a method for performing a high throughput genotyping assay to determine an allele status of more than one genomic loci in a group of test subjects. In some embodiments, the method includes: (1) dispensing reaction mixes into a plurality of single in vitro reaction vessels, (2) amplifying a plurality of genomic loci in a sample in the presence of fluorescently-labeled probes, (3) detecting fluorescent signal from the probes corresponding to the particular alleles present in the sample, and (4) either reporting the status of the alleles when no variant alleles are present or performing secondary allele detection assays when at least one less prevalent allele is present in the sample.

A. High-Throughput Multiplexing for a First Plurality of Genomic Loci

In some embodiments, a method is provided for performing a high throughput genotyping assay. The method includes dispensing, into each respective well in a first plurality of wells in a multiwell plate, according to one or more template plate definitions associated with the high throughput genotyping assay, a respective template nucleic acid preparation, reagents for amplifying a first plurality of genomic loci, and a first plurality of fluorescently-labeled detection reagents. The respective template nucleic acid preparation dispensed into each respective well is prepared from a respective biological sample obtained from a different test subject in a plurality of test subjects. The first plurality of fluorescently-labeled detection reagents includes, for each respective genomic locus in the first plurality of genomic loci, a first detection reagent specific for the presence of a first allele at the respective genomic locus, where the first allele is the most prevalent allele in a population of the species of the subject and the first detection reagent is labeled with a first fluorescent moiety. The first plurality of fluorescently-labeled detection reagents also includes, for the same respective genomic locus in the first plurality of genomic loci, a second detection reagent specific for the presence of a second allele at the respective genomic locus, where the second allele is a minor allele in the population and the second detection reagent is labeled with a second fluorescent moiety distinguishable from the first fluorescent moiety. The method then includes by amplifying the first plurality of genomic loci in each respective well after the dispensing. The method then includes by detecting in each respective well a fluorescent signal corresponding to the first fluorescent moiety and the second fluorescent moiety in the reaction vessel, during or after the amplifying. When the contribution of the second fluorescent moiety to the fluorescent signal detected in the respective well does not satisfy a threshold contribution, the corresponding subject in the plurality of subjects is reported to not carry the second allele at any of the first plurality of genomic loci. When the contribution of the second fluorescent moiety to the fluorescent signal detected in the respective well satisfies the threshold contribution, a first plurality of secondary allele detection assays using a template nucleic acid preparation from the corresponding subject in the plurality of subjects is performed, where each secondary allele detection assay in the first plurality of secondary allele detection assays determines the allele status at one respective genomic locus in the first plurality of genomic loci, and the allele status of the corresponding subject at each of the first plurality of genomic loci is reported based on the first plurality of secondary allele detection assays.

1. Dispensing

In some embodiments, the methods described herein include dispensing, into respective well in a first plurality of wells in a multiwell plate, following one or more template plate definitions associated with the high throughput genotyping assay, a respective template nucleic acid preparation, reagents for amplifying a first plurality of genomic loci, and a first plurality of fluorescently-labeled detection reagents. The respective template nucleic acid preparation dispensed into each respective well is prepared from a respective biological sample obtained from a different test subject in a plurality of test subjects. The first plurality of fluorescently-labeled detection reagents includes, for each respective genomic locus in the first plurality of genomic loci, (i) a first detection reagent that is specific for the presence of a first allele at the respective genomic locus, where the first allele is the most prevalent allele in a population of the species of the subject and the first detection reagent is labeled with a first fluorescent moiety, and (ii) a second detection reagent that is specific for the presence of a second allele at the respective genomic locus, where the second allele is a minor allele in the population and the second detection reagent is labeled with a second fluorescent moiety that is distinguishable from the first fluorescent moiety.

In one embodiment, the multiwell plate is a 96-well plate. In other embodiments, the multiwell plate is a 384-well plate, a 1536-well plate, a 3456-well plate, or a 9600-well plate. In yet another embodiment, the multiwell plate is a microchip array. The type of plate used in the reaction should be determined based on the number of samples being tested and compatibility with any automated liquid handlers being used to dispense the reagents and the instrumentation used for the reaction and fluorescent detection. In some embodiments, an automated liquid handler is used to dispense the reagents into the multiwell plate. In other embodiments, multi-channel pipettes and/or microfluidic channels are used for dispensing.

In some embodiments, the reagents for amplifying a first plurality of genomic loci comprises TaqMan® Genotyping Master Mix. In another non-limiting embodiment, the reagents for amplifying a first plurality of genomic loci comprises TaqMan® Genotyping Assay Mixes.

In some embodiments, the first plurality of genomic loci include at least two genomic loci. In some embodiments, the first plurality of genomic loci include at least three genomic loci. In some embodiments, the first plurality of genomic loci include at least four, five, six, seven, eight, nine, ten, or more genomic loci.

In some embodiments, the genomic loci are associated with a particular condition or a class of related conditions, e.g., neuropsychiatric disorders, diabetes, cardiovascular disease (e.g., heart disease), hypertension, Alzheimer's disease, cancer, obesity, arthritis, or asthma. In one embodiment, the genomic loci are associated with alleles known to have pharmacogenetic effects, such that the results of the assay inform treatment of the underlying condition. For instance, in some embodiments, the genomic loci are associated with neuropsychiatric disorders, for instance with alleles at which SNPs having pharmacogenetic effects on various therapies used to treat neuropsychiatric disorders have been identified. Non-limiting examples of such SNPs having pharmacogenetic effects on various therapies used to treat neuropsychiatric disorders, and the loci they are associated with, are provided in Table 1 and/or Table 2, below. In some embodiments, the genomic loci amplified in the methods described herein are selected from those loci shown in Table 1 and/or Table 2. In some embodiments, the genomic loci include at least four, five, six, seven, eight, nine, ten, or more genomic loci listed in Table 1 and/or Table 2. Further description of the pharmacogenetic effects, and the therapy recommendations derived therefrom, are provided in the section titled “Single nucleotide polymorphism (SNP) markers,” below.

In one embodiment, the first plurality of genomic loci include the human alleles corresponding to the SNPs rs5030863, rs28371685, and rs5030867. Specifically, rs5030863 is a SNP at position 42525912 of human chromosome 22, in the CYP2D6 gene. The presence of SNP rs5030863 is associated with inactivity of P450 2D6. Accordingly, in some embodiments, the methods provided herein further include administering, to the subject, a low dose of a pharmaceutical agent that is metabolized by cytochrome P450 2D6 (CYP2D6) when the subject is determined to carry the rs5030863 SNP. rs28371685 is a SNP at position 94981224 of human chromosome 10, in the CYP2C9 gene. The presence of SNP rs28371685 is associated with reduced activity of P450 2C9. Accordingly, in some embodiments, the methods provided herein further include administering, to the subject, a low dose of a pharmaceutical agent that is metabolized by cytochrome P450 2C9 (CYP2C9) when the subject is determined to carry the rs28371685 SNP. rs5030867 is a SNP at position 42127856 of human chromosome 22, in the CYP2D6 gene. The presence of SNP rs5030867 is associated with inactivity of P450 2D6. Accordingly, in some embodiments, the methods provided herein further include administering, to the subject, a low dose of an antipsychotic drug when the subject is determined to carry the rs5030867 SNP.

In one embodiment, the first plurality of genomic loci include the human alleles corresponding to the SNPs rs17884712, rs72552267, and rs72558187. Specifically, rs17884712 is a SNP at position 94775489 of human chromosome 10, in the CYP2C19 gene. The presence of SNP rs17884712 is associated with reduced activity of P450 2C19. Accordingly, in some embodiments, the methods provided herein further include administering, to the subject, a low dose of a pharmaceutical agent that is metabolized by cytochrome P450 2C19 (CYP2C19) when the subject is determined to carry the rs17884712 SNP. rs72552267 is a SNP at position 94775453 of human chromosome 10, in the CYP2C19 gene. The presence of SNP rs72552267 is associated with reduced activity of P450 2C19. Accordingly, in some embodiments, the methods provided herein further include administering, to the subject, a low dose of a pharmaceutical agent that is metabolized by cytochrome P450 2C19 (CYP2C19) when the subject is determined to carry the rs72552267 SNP. rs72558187 is a SNP at position 94941958 of human chromosome 10, in the CYP2C9 gene. The presence of SNP rs72558187 is associated with reduced activity of P450 2C9. Accordingly, in some embodiments, the methods provided herein further include administering, to the subject, a low dose of a pharmaceutical agent that is metabolized by cytochrome P450 2C9 (CYP2C9) when the subject is determined to carry the rs72558187 SNP.

In one embodiment, the first plurality of genomic loci include the human alleles corresponding to the SNPs rs5030862 and rs56337013. Specifically, rs5030862 is a SNP at position 42130668 of human chromosome 22, in the CYP2D6 gene. The presence of SNP rs5030862 is associated with inactivity of P450 2D6. Accordingly, in some embodiments, the methods provided herein further include administering, to the subject, a low dose of a pharmaceutical agent that is metabolized by cytochrome P450 2D6 (CYP2D6) when the subject is determined to carry the rs5030862 SNP. rs56337013 is a SNP at position 94852738 of human chromosome 10, in the CYP2C19 gene. The presence of SNP rs56337013 is associated with reduced activity of P450 2C19. Accordingly, in some embodiments, the methods provided herein further include administering, to the subject, a low dose of a pharmaceutical agent that is metabolized by cytochrome P450 2C19 (CYP2C19) when the subject is determined to carry the rs56337013 SNP.

Generally, in order for the multiplex reaction to reduce the total number of detection reactions needed, the wild type allele must be prevalent enough in the population such that identification of a variant allele will be a fairly rare occurrence. Accordingly, in some embodiments, for each respective genomic locus in the first plurality of genomic loci, the frequency of the first allele in the population is at least 80%. In some embodiments, for each respective genomic locus in the first plurality of genomic loci, the frequency of the first allele in the population is at least 85%. In some embodiments, for each respective genomic locus in the first plurality of genomic loci, the frequency of the first allele in the population is at least 90%. In some embodiments, for each respective genomic locus in the first plurality of genomic loci, the frequency of the first allele in the population is at least 95%. In some embodiments, for each respective genomic locus in the first plurality of genomic loci, the frequency of the first allele in the population is at least 96%, 97%, 98%, or 99%. Similarly, in some embodiments, for each respective genomic locus in the first plurality of genomic loci, the frequency of the second allele in the population is no more than 20%, or no more than 15%, 10%, 5%, 4%, 3%, 2%, or 1%. Accordingly, in some embodiments, the combined frequency, in the population, of the second allele for each respective loci in the first plurality of loci is no more than 40%. In some embodiments, the combined frequency, in the population, of the second allele for each respective loci in the first plurality of loci is no more than 30%. In some embodiments, the combined frequency, in the population, of the second allele for each respective loci in the first plurality of loci is no more than 20%. In some embodiments, the combined frequency, in the population, of the second allele for each respective loci in the first plurality of loci is no more than 10%. In some embodiments, the combined frequency, in the population, of the second allele for each respective loci in the first plurality of loci is no more than 5%, 4%, 3%, 2%, or 1%.

Accordingly, in some embodiments, for each respective genomic locus in the first plurality of genomic loci, the first detection reagent comprises a first oligonucleotide labeled with a first matching pair of electronic energy transfer chromophores, wherein the sequence of the first oligonucleotide is complementary to the first allele at the respective genomic locus. For each respective genomic locus in the first plurality of genomic loci, the second detection reagent comprises a second oligonucleotide labeled with a second matching pair of electronic energy transfer chromophores, wherein the sequence of the second oligonucleotide is complementary to the second allele at the respective genomic locus.

In some embodiments, the emission spectra of fluorescent signal corresponding to the first and the second excitation chromophores are distinguishable enough in wavelength so as to allow independent detection for each excitation chromophore. In other embodiments, the emission spectra of the first and the second excitation chromophores overlap to the extent that the detection of fluorescence signal from both chromophores can be combined into one single detection. In a specific embodiment, the wavelength centers of emission spectra of the first and the second excitation chromophores are within 20 nm or less, or within 15 nm, 10 nm, 5 nm, or less.

In some embodiments, for each respective genomic locus in the first plurality of genomic loci, the first matching pair of electronic energy transfer chromophores consists of a first excitation chromophore and a first quenching chromophore for the first excitation chromophore; and the second matching pair of electronic energy transfer chromophores consists of a second excitation chromophore and a second quenching chromophore for the second excitation chromophore. Electronic energy transfer chromophores, as well as excitation and quenching effect, are commonly known to a person of ordinary skill in the art, and their description is provided in subsequent sections.

In one specific embodiment, for each respective genomic locus in the first plurality of genomic loci: one of the first matching pair of electronic energy transfer chromophores or the second matching pair of electronic energy transfer chromophores consists of a 6-carboxyfluorescein excitation chromophore and a 5-carboxytetramethylrhodamine quenching chromophore. The other of the first matching pair of electronic energy transfer chromophores or the second matching pair of electronic energy transfer chromophores consists of a 2′-chloro-7′phenyl-1,4-dichloro-6-carboxy-fluorescein excitation chromophore and a 5-carboxytetramethylrhodamine quenching chromophore. The selection of matching pair of EET chromophores is described in detail in subsequent sections.

In some embodiments, the plurality of fluorescently-labeled detection reagents are configured for a TaqMan® assay. These fluorescently-labeled detection reagents (probes) for TaqMan® assays are well-known in the art. A detailed description of the probes are provided in subsequent sections. However, generally, these probes include a polynucleotide with a sequence that is specific for a first allele at the locus of interest having a fluorescent moiety and a quencher moiety attached to the polynucleotide at a distance apart that is sufficient to allow for quenching of the fluorescent moiety by the quencher.

2. Amplifying Target Loci

The methods then includes, after dispensing, amplifying the first plurality of genomic loci in each respective well. In some embodiments, amplifying a plurality of genomic loci includes performing a polymerase chain reaction (PCR) on a sample of nucleic acids that include the plurality of genomic loci to increase the copy number of the plurality of genomic loci. PCR techniques that can be used include but are not limited to digital PCR (dPCR), quantitative PCR (qPCR) or real-time PCR (e.g., TaqMan PCR; Applied Biosystems), reverse-transcription PCR (RT-PCR), allele-specific PCR, amplified fragment length polymorphism PCR (AFLP PCR), colony PCR, Hot Start PCR, in situ PCR (ISH PCR), inverse PCR (IPCR), long PCR, multiplex PCR, or nested PCR. As described above, PCR techniques for amplifying target loci are well known in the art, and one of skill in the art is capable of applying PCR to the method described herein, including identifying proper reaction reagents, ascertaining optimal reaction conditions and parameters, and making necessary modifications. Examples of guidelines and protocols for PCR include: Innis et al., (2012), PCR protocols: a guide to methods and applications, Academic press; and Logan et al., (2009), Real-time PCR: current technology and applications, Horizon Scientific Press

3. Detecting Fluorescent Signal

The methods then include detecting, during or after the amplifying, in each respective well, a fluorescent signal corresponding to the first fluorescent moiety and the second fluorescent moiety in the reaction vessel.

In some embodiments, the detecting occurs during the amplifying. For instance, in some embodiments, the reaction is performed using a quantitative PCR thermal cycler. In some embodiments, the quantitative PCR thermal cycler uses an LED light source to excite the fluorescent moieties at excitation wavelengths, and then measures the emission at corresponding emission wavelengths. In some embodiments, e.g., where a TaqMan® reaction is employed, the detection of an emission wavelength indicates that the fluorescent moiety has been physically separated from the quencher moiety, evidencing the presence of the target allele in the assay sample.

In some embodiments, the detecting occurs after the amplifying. For instance, in some embodiments, the amplifying is performed in a thermal cycler, which may or may not be a quantitative PCR thermal cycler, and the product is evaluated for presence of the probe fluorophores, e.g., where detection of the emission wavelength indicates the presence of the target allele in the assay sample.

In some embodiments, the methods described herein reduce the time needed to genotype a plurality of loci from a plurality of subjects, by performing a plurality of multiplexing reactions in a single multiwell plate. In some embodiments, a plurality of loci are genotyped from at least 50, 100, 250, 500, 750, 1000, 2500, 5000, of more subjects in a single run. Accordingly, in some embodiments, the plurality of loci from the plurality of subjects are genotyped, e.g., the dispensing, amplifying, and detecting are performed, within twelve hours, or within six hours, or within five hours, or within four hours, or within three hours.

4. Reporting Allele Status

The methods then include, responsive to the detecting, for each respective well (i) when the contribution of the second fluorescent moiety to the fluorescent signal detected in the respective well does not satisfy a threshold contribution, reporting that the corresponding subject in the plurality of subjects does not carry the second allele at any of the first plurality of genomic loci, and (ii) when the contribution of the second fluorescent moiety to the fluorescent signal detected in the respective well satisfies the threshold contribution, performing a first plurality of secondary allele detection assays using a template nucleic acid preparation from the corresponding subject in the plurality of subjects, where each secondary allele detection assay in the first plurality of secondary allele detection assays determines the allele status at one respective genomic locus in the first plurality of genomic loci, and reporting the allele status of the corresponding subject at each of the first plurality of genomic loci based on the first plurality of secondary allele detection assays.

The threshold contribution is a level of fluorescence, detected from fluorophores that correspond to variant alleles, that indicates the presence of one or more variant alleles at a loci being tested. As described above, the threshold level is set based on considerations such as, the number of alleles being tested, the relative efficiencies of all fluorophores used in the reaction, the relative sensitivity of the detectors used for the fluorophores, the expected ratio of alleles represented in the sample, whether the presence of alternate alleles is desired, etc. Although each probe for a particular locus is specific for one allele or the other, the identity in the probe region flanking the SNP results in some cross-hybridization, such that some baseline signal from the second fluorophore is expected even when the sample does not contain a variant allele. Accordingly, the threshold contribution should be set to account for this baseline signal.

Accordingly, in some embodiments, the threshold contribution is set such that it is satisfied when the sample includes nucleic acids from a subject carrying a variant allele in at least one locus being tested. For instance, the contribution of the second fluorescent moiety to the fluorescent signal detected in the reaction vessel satisfies the threshold contribution when the contribution of the second fluorescent moiety to the fluorescent signal indicates that, for at least one respective genomic locus in the plurality of genomic loci, the first allele is not present in at least one half of the nucleic acids encompassing the respective loci. For example, where the reaction includes nucleic acids from a single subject, and three different loci are being tested, the threshold contribution is set such that it is satisfied when at least one sixth of the total fluorescent signal, accounting for variables as discussed above, is attributable to the second fluorophore.

Similarly, in some embodiments, the contribution of the second fluorescent moiety to the fluorescent signal detected in the reaction vessel does not satisfy the threshold contribution when the contribution of the second fluorescent moiety to the fluorescent signal indicates that, for each respective genomic locus in the plurality of genomic loci, the first allele is present in more than one half of the nucleic acids encompassing the respective loci. That is, the threshold contribution is satisfied when the subject does not carry a variant allele at any of the loci being tested.

In some embodiments, when at least one of the subject's two copies of a respective genomic locus carries a third allele, i.e., an allele other than the first allele or the second allele, the first probe and the second probe will bind to the nucleic acids encompassing the third allele with similar affinity, such that approximately a quarter of the fluorescent signal attributable to the loci will be from the second detection probe, assuming that the other copy of the loci contains the first (most prevalent) allele. Accordingly, in some embodiments, e.g., when it is desirable to detect the presence of a third allele at a locus, the threshold contribution is set such that it is satisfied when at least one quarter of the fluorescent signal from any locus being tested is attributable to the second fluorophore. For example, where the reaction includes nucleic acids from a single subject, and three different loci are being tested, the threshold contribution is set such that it is satisfied when at least one twelfth of the total fluorescent signal, accounting for variables as discussed above, is attributable to the second fluorophore.

In some embodiments, the threshold contribution of the second fluorescent moiety is determined by referring to the contribution of the second fluorescent moiety to the fluorescent signal corresponding to both a positive control subject and a negative control subject.

When the contribution of the second fluorescent moiety to the fluorescent signal detected in the reaction vessel satisfies the threshold contribution, the allele status of each genomic locus is then determined individually. This is because the multiplex assay is unable to determine which of the loci carries the variant allele, since each variant allele is associated with the same fluorophore (i.e., the second fluorophore). These secondary allele detection assays can be performed according to any known method for determining the identity of an allele, including sequencing, qPCR, hybridization, and TaqMan® assay. In some embodiments, the secondary allele detection assays are performed using non-multiplexed TaqMan® assays.

In some embodiments, when it is determined that a subject carries a minor allele at a respective genomic locus in the plurality of genomic loci that is associated with a pharmacogenetic effect, a warning, precaution, or drug interaction for a pharmaceutical agent associated with the minor allele of the respective loci is reported, e.g., to a healthcare professional and/or directly to the subject.

B. High-Throughput Multiplexing for a Second Plurality of Genomic Loci

In some embodiments, a second plurality of loci for each subject can be tested in a similar high-throughput fashion as the as the first plurality of loci, by using a second plurality of wells. In such fashion, an even larger number of loci can be tested at a single time.

Accordingly, in some embodiments of the dispensing step described above, a respective template nucleic acid preparation, reagents for amplifying a second plurality of genomic loci, and a second plurality of fluorescently-labeled detection reagents are dispensed into each respective well in a second plurality of wells in a multiwell plate, in accordance with the one or more template plate definitions associated with the high throughput genotyping assay. The respective template nucleic acid preparation dispensed into each respective well is prepared from a respective biological sample obtained from a different test subject in the plurality of test subjects. The second plurality of fluorescently-labeled detection reagents includes, for each respective genomic locus in the second plurality of genomic loci, (i) a third detection reagent that is specific for the presence of a first allele at the respective genomic locus, where the first allele is the most prevalent allele in a population of the species of the subject and the third detection reagent is labeled with a third fluorescent moiety, and (ii) a fourth detection reagent that is specific for the presence of a second allele at the respective genomic locus, where the second allele is a minor allele in the population and the fourth detection reagent is labeled with a second fluorescent moiety that is distinguishable from the third fluorescent moiety.

In some embodiments, the first plurality of wells and the second plurality of wells are in the same multiwell plate. In other embodiments, the first plurality of wells and the second plurality of wells are in different multiwell plates.

In some embodiments, the second plurality of genomic loci include at least two genomic loci. In some embodiments, the second plurality of genomic loci include at least three genomic loci. In some embodiments, the second plurality of genomic loci include at least four, five, six, seven, eight, nine, ten, or more genomic loci.

In some embodiments, the second plurality of genomic loci are associated with a same particular condition or a class of related conditions as the first plurality of loci, e.g., neuropsychiatric disorders, diabetes, cardiovascular disease (e.g., heart disease), hypertension, Alzheimer's disease, cancer, obesity, arthritis, or asthma. In some embodiments, the second plurality of loci include the human alleles corresponding to SNPs rs5030863, rs28371685, and rs5030867. In another embodiment, the second plurality of genomic loci includes the human alleles corresponding to SNPs rs17884712, rs72552267, and rs72558187. In another embodiment, the second plurality of genomic loci includes the human alleles corresponding to SNPs rs5030862 and rs56337013.

Similarly, in some embodiments of the amplifying step described above includes amplifying the second plurality of genomic loci in each respective well in the second plurality of wells. In some embodiments, the second plurality of genomic loci are amplified at the same time as the first plurality of genomic loci, e.g., when present in the same multiwell plate or in a different multiwell plate. During or after the amplifying, the method includes detecting, in each respective well, a fluorescent signal corresponding to the third fluorescent moiety and the fourth fluorescent moiety. In some embodiments, the third fluorescent moiety and the fourth fluorescent moiety are the same as the first fluorescent moiety and the second fluorescent moiety. This is possible because the first plurality of loci and the second plurality of loci are assayed in different wells.

Likewise, in some embodiments of the reporting step described above, responsive to the detecting, (i) when the contribution of the fourth fluorescent moiety to the fluorescent signal detected in a respective well does not satisfy a threshold contribution, the method includes reporting that the subject does not carry the second allele at any of the second plurality of genomic loci, and (ii) when the contribution of the fourth fluorescent moiety to the fluorescent signal detected in a respective well satisfies the threshold contribution, the method includes performing a second plurality of secondary allele detection assays, wherein each secondary allele detection assay in the second plurality of secondary allele detection assays determines the allele status at one respective genomic locus in the second plurality of genomic loci, and reporting the allele status at each of the second plurality of genomic loci based on the second plurality of secondary allele detection assays.

### III. Multiplexing for Managing Neuropsychiatric Disorders

Treatment guidelines for some SMIs, such as MDD, offer little to distinguish among first-line treatment options. APA, Practice Guideline for the Treatment of Major Depressive Disorder (3rd ed. 2010). A recent meta-analysis described comparable efficacy and lack of superiority for any of a range of antidepressants. Cipriani A. et al., Lancet, 391(10128):1357-66 (2018). Rates of remission for all of these interventions are modest, estimated at ˜1 in 3. Trivedi M H et al., Am J Psych, 163:28-40 (2006). However, available drugs differ in their potential to cause adverse drug effects. Without having patient-specific genetic variation information, physicians often base initial selection upon clinician experience and comfort, cost, known general tolerability profile, the presence of concomitant co-morbidities and medications, and patient preference (for example based on concerns about weight gain or sexual dysfunction). In the largest and longest evaluation of antidepressants, the Sequenced Treatment Alternatives to Relieve Depression (STAR*D) trial, it took more than 50 weeks and at least four sequential courses of therapy to obtain a cumulative remission rate of 67%. The STAR*D trial also demonstrated that the highest likelihood of remission with antidepressants is following the first or second drug selected by the provider and falls below 15% with subsequent course of treatment. Using genetic variant data to identify initial medications with a more favorable patient-specific tolerability, and ruling out drugs that are less likely to be well-tolerated, or would require dose adjustments based upon label guidance would be highly beneficial.

The individual and societal consequences of ineffective or poorly tolerated drugs in SMI patients are profound, and include adverse drug effects, low adherence, unnecessary medical expenses, decreased quality of life, and increased mortality. A method that improved the selection of appropriate pharmacotherapy for an individual with SMI would be very desirable because it would lead to more effective and/or better-tolerated treatment. The pharmacoeconomic advantage of pharmacogenetic testing has been demonstrated repeatedly, including in patients with SMI, such as MDD and schizophrenia. Fagerness J. et al., Am J Manag Care, 20(5):e146-56 (2014); Perlis R H et al., Depress Anxiety, 35(10):946-52 (2018); and Herbild L. et al., Basic Clin Pharmacol Toxicol, 113(4):266-72 (2013).

Advantageously, the methods described herein provide clinicians with information on the biomarker genetic variants listed in Table 1 and/or Table 2. Although there are warnings, precautions, and/or drug interaction statements in various drug labels regarding these associations, these biomarkers are often not measured. Currently, there are no FDA-cleared tests that include all of the relevant genes in one assay. Thus, even though FDA has determined that it is clinically informative to have this information, it cannot currently be readily obtained. Moreover, even though there are tests for several of these genes, they are for single genes, most of which require whole blood for testing. Furthermore, clinicians often consider multiple pharmaceutical options for a single patient. Having all of these gene-drug associations and results in a single assay, readily obtained in a clinical setting, e.g., via buccal swab, would facilitate treatment decisions by enabling physicians to have access to information that FDA has deemed clinically important. However, given the growing number of known pharmacogenetic associations, conventional detection methodologies, even if clinically validated assay for each SNP existed, would not be economically feasible for a medical professional to obtain a full panel of associations for a patient.

Accordingly, one aspect of the disclosure provides a method for providing treatment guidance for a neuropsychiatric disorder in a subject. In some embodiments, the neuropsychiatric disorder is major depression, anxiety disorder, obsessive-compulsive disorder, attention deficit hyperactivity disorder (ADHD), bipolar disorder, post-traumatic stress disorder (PTSD), autism, schizophrenia, personality disorder, chronic pain, substance abuse, or any combination thereof. In some embodiments, the method includes (A) determining the allele status for a plurality of genomic loci, (B) associating the allele status determined for the plurality of genomic loci with one or more recommendations for the treatment of the neuropsychiatric disorder, and (C) generating a patient-specific report comprising the one or more recommendations for the treatment of the neuropsychiatric disorder.

In some embodiments, the allele statuses are determined using the multiplex reaction methods described above. For the sake of brevity, a thorough description of these multiplexing methods is not repeated here. Rather, the methods, including each variation described above, is incorporated into this section, in their entireties, as if they were repeated verbatim. The skilled artisan will recognize that the methods above are as equally applicable for the detection of alleles associated with neuropsychiatric disorders, e.g., known to have pharmacogenetic associations with various treatments used to manage neuropsychiatric disorders, as for any other group of alleles.

A. Analysis of a First Set of Genomic Loci

In one embodiment, a method for providing guidance for the treatment of a neuropsychiatric disorder in a subject, e.g., a human, is provided. The method includes determining the allele status for a plurality of genomic loci, wherein each respective loci in the plurality of loci is associated with a therapeutic efficacy of at least one therapy for a neuropsychiatric disorder. In some embodiments, the determining is performed by amplifying, in a first single in vitro reaction vessel, a first set of two or more genomic loci in the plurality of genomic loci, by polymerase chain reaction (PCR), from nucleic acids isolated from a sample obtained from the subject. As described above, in some embodiments, the first set of genomic loci is at least three loci, or at least four, five, six, seven, eight, nine, ten, or more genomic loci. In some embodiments, as described above, the sample obtained from the subject comprises buccal cells, saliva, or blood.

The amplification is performed in the presence of a plurality of fluorescently-labeled detection reagents, e.g., as described above. The plurality of fluorescently-labeled detection reagents includes, for each respective genomic locus in the two or more genomic loci (i) a first detection reagent that is specific for the presence of a first allele at the respective genomic locus, where the first allele is the most prevalent allele in a population of the species of the subject and the first detection reagent is labeled with a first fluorescent moiety, and (ii) a second detection reagent that is specific for the presence of a second allele at the respective genomic locus, where the second allele is a minor allele in the population and the second detection reagent is labeled with a second fluorescent moiety that is distinguishable from the first fluorescent moiety. As described above, in some embodiments, for each respective genomic locus in the first plurality of genomic loci, the frequency of the first allele in the population is at least 80%, 90%, 95%, or higher. Similarly, as described above, in some embodiments, for each respective genomic locus in the first plurality of genomic loci, the frequency of the second allele in the population is no more than 20%, 10%, 5%, or less.

In some embodiments, the first set of two or more genomic loci include two or more of the genomic loci listed in Table 1 and/or Table 2. In some embodiments, the genomic loci include at least four, five, six, seven, eight, nine, ten, or more genomic loci listed in Table 1 and/or Table 2. Further description of the pharmacogenetic effects, and the therapy recommendations derived therefrom, are provided in the section titled “Single nucleotide polymorphism (SNP) markers,” below.

During or after the amplifying, the method includes detecting a fluorescent signal corresponding to the first fluorescent moiety and the second fluorescent moiety in the first single reaction vessel. Responsive to the detecting, when the contribution of the second fluorescent moiety to the fluorescent signal detected in the reaction vessel does not satisfy a threshold contribution, determining that the subject is homozygous for the first allele at each respective loci in the first set of two or more genomic loci, and when the contribution of the second fluorescent moiety to the fluorescent signal detected in the reaction vessel satisfies the threshold contribution, performing a first plurality of secondary allele detection assays, wherein each secondary allele detection assay in the first plurality of secondary allele detection assays determines the allele status at one respective genomic locus in the first set of two or more genomic loci, thereby determining the allele status at each respective loci in the first set of two or more genomic loci.

In some embodiments, the method then includes associating the allele status determined for the plurality of genomic loci with one or more recommendations for the treatment of a neuropsychiatric disorder. In some embodiments, this is performed by a suitably programed computer, e.g., having a look-up table with known pharmacogenetic associations for each variant allele detectable by the assay. Example systems for performing this step are described in more detail below.

In some embodiments, the method then includes generating a patient-specific report comprising the one or more recommendations for the treatment of the neuropsychiatric disorder. In some embodiments, this is again performed by a suitably programed computer. Example systems for performing this step are described in more detail below.

B. Analysis of a Second Set of Genomic Loci

In some embodiments, a second set of loci can be tested in a similar fashion as the first set of loci, using a second reaction vessel. In such fashion, an even larger number of loci can be tested at a single time.

Accordingly, in some embodiments of the amplifying step described above, a second set of two or more genomic loci in the plurality of genomic loci are amplified, in a second single in vitro reaction vessel, by polymerase chain reaction (PCR), from nucleic acids isolated from a sample obtained from the subject, in the presence of a plurality of fluorescently-labeled detection reagents. The plurality of fluorescently-labeled detection reagents includes, for each respective genomic locus in the second set of two or more genomic loci, (i) a third detection reagent that is specific for the presence of a first allele at the respective genomic locus, where the first allele is the most prevalent allele in a population of the species of the subject and the third detection reagent is labeled with a third fluorescent moiety, and (ii) a fourth detection reagent that is specific for the presence of a second allele at the respective genomic locus, wherein the second allele is a minor allele in the population and the fourth detection reagent is labeled with a fourth fluorescent moiety that is distinguishable from the third fluorescent moiety.

During or after the amplifying, the method includes detecting a fluorescent signal corresponding to the third fluorescent moiety and the fourth fluorescent moiety in the second single reaction vessel. In some embodiments, the third fluorescent moiety and the fourth fluorescent moiety are the same as the first fluorescent moiety and the second fluorescent moiety. This is possible because the first plurality of loci and the second plurality of loci are assayed in different reaction vessels. In some embodiments, the third fluorescent moiety and the fourth fluorescent moiety are different than the first fluorescent moiety and the second fluorescent moiety.

Likewise, in some embodiments of the reporting step described above, responsive to the detecting, (i) when the contribution of the fourth fluorescent moiety to the fluorescent signal detected in the reaction vessel does not satisfy a threshold contribution, determining that the subject is homozygous for the first allele at each respective loci in the second set of two or more loci, and (ii) when the contribution of the fourth fluorescent moiety to the fluorescent signal detected in the reaction vessel satisfies the threshold contribution, performing a second plurality of secondary allele detection assays, where each secondary allele detection assay in the second plurality of secondary allele detection assays determines the allele status at one respective genomic locus in the second set of two or more genomic loci, thereby determining the allele status at each respective loci in the second set of two or more genomic loci.

C. Analysis of a Third Set of Genomic Loci

In some embodiments, a third set of loci can be tested in a similar fashion as the first set of loci and second set of loci, using a third reaction vessel. In such fashion, an even larger number of loci can be tested at a single time.

Accordingly, in some embodiments of the amplifying step described above, a third set of two or more genomic loci in the plurality of genomic loci are amplified, in a third single in vitro reaction vessel, by polymerase chain reaction (PCR), from nucleic acids isolated from a sample obtained from the subject, in the presence of a plurality of fluorescently-labeled detection reagents. The plurality of fluorescently-labeled detection reagents includes, for each respective genomic locus in the third set of two or more genomic loci, (i) a fifth detection reagent that is specific for the presence of a first allele at the respective genomic locus, where the first allele is the most prevalent allele in a population of the species of the subject and the fifth detection reagent is labeled with a fifth fluorescent moiety, and (ii) a sixth detection reagent that is specific for the presence of a second allele at the respective genomic locus, wherein the second allele is a minor allele in the population and the sixth detection reagent is labeled with a sixth fluorescent moiety that is distinguishable from the fifth fluorescent moiety.

During or after the amplifying, the method includes detecting a fluorescent signal corresponding to the fifth fluorescent moiety and the sixth fluorescent moiety in the second single reaction vessel. In some embodiments, the fifth fluorescent moiety and the sixth fluorescent moiety are the same as the first fluorescent moiety and the second fluorescent moiety. This is possible because the first plurality of loci and the third plurality of loci are assayed in different reaction vessels. In some embodiments, the fifth fluorescent moiety and the sixth fluorescent moiety are different from the first fluorescent moiety and the second fluorescent moiety.

Likewise, in some embodiments of the reporting step described above, responsive to the detecting, (i) when the contribution of the sixth fluorescent moiety to the fluorescent signal detected in the reaction vessel does not satisfy a threshold contribution, determining that the subject is homozygous for the first allele at each respective loci in the third set of two or more loci, and (ii) when the contribution of the sixth fluorescent moiety to the fluorescent signal detected in the reaction vessel satisfies the threshold contribution, performing a third plurality of secondary allele detection assays, where each secondary allele detection assay in the third plurality of secondary allele detection assays determines the allele status at one respective genomic locus in the third set of two or more genomic loci, thereby determining the allele status at each respective loci in the third set of two or more genomic loci.

D. Genomic Loci Associated with Neuropsychiatric Disorders

In some embodiments, the plurality of genomic loci associated with a neuropsychiatric disorder includes one or more genomic loci corresponding to a SNP selected from the group consisting of rs7997012, rs3813929, rs1045642, rs2032583, rs1800544, rs10994336, rs6265, rs1006737, rs4680, rs2470890 (CYP1A2*1B), rs2069514, rs35694136, rs2069526 (CYP1A2*1E), rs762551, rs12720461 (CYP1A2*1K), rs2069526 (CYP1A2*1K), rs72547513, rs2279343 (CYP2B6*4), rs3211371, rs3745274 (CYP2B6*6), rs2279343 (CYP2B6*6), rs4244285, rs17878459 (CYP2C19*2B), rs4986893 (CYP2C19*3), rs57081121 (CYP2C19*3), rs28399504, rs56337013, rs72552267, rs72558186, rs41291556, rs17884712, rs6413438, rs12248560, rs12769205 (CYP2C19*35), rs3758581 (CYP2C19*35), rs1799853, rs1057910, rs56165452, rs28371686, rs9332131, rs7900194, rs28371685, rs72558187, rs7900194 (CYP2C9*27), rs16947 (CYP2D6*2), rs1135840 (CYP2D6*2), rs1135824 (CYP2D6*3), rs35742686 (CYP2D6*3), rs3892097, rs5030655, rs5030867, rs5030865, rs5030656, rs1065852, rs5030863, rs5030862, rs5030865, rs774671100, rs28371706 (CYP2D6*17), rs16947 (CYP2D6*17), rs61736512 (CYP2D6*29), rs1058164 (CYP2D6*29), rs16947 (CYP2D6*29), rs59421388 (CYP2D6*29), rs1135840 (CYP2D6*29), rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023.

In some embodiments, the plurality of genomic loci associated with a neuropsychiatric disorder includes at least the genomic loci corresponding to SNPs rs7997012, rs3813929, rs1045642, rs2032583, rs1800544, rs10994336, rs6265, rs1006737, rs4680, rs2470890 (CYP1A2*1B), rs2069514, rs35694136, rs2069526 (CYP1A2*1E), rs762551, rs12720461 (CYP1A2*1K), rs2069526 (CYP1A2*1K), rs72547513, rs2279343 (CYP2B6*4), rs3211371, rs3745274 (CYP2B6*6), rs2279343 (CYP2B6*6), rs4244285, rs17878459 (CYP2C19*2B), rs4986893 (CYP2C19*3), rs57081121 (CYP2C19*3), rs28399504, rs56337013, rs72552267, rs72558186, rs41291556, rs17884712, rs6413438, rs12248560, rs12769205 (CYP2C19*35), rs3758581 (CYP2C19*35), rs1799853, rs1057910, rs56165452, rs28371686, rs9332131, rs7900194, rs28371685, rs72558187, rs7900194 (CYP2C9*27), rs16947 (CYP2D6*2), rs1135840 (CYP2D6*2), rs1135824 (CYP2D6*3), rs35742686 (CYP2D6*3), rs3892097, rs5030655, rs5030867, rs5030865, rs5030656, rs1065852, rs5030863, rs5030862, rs5030865, rs774671100, rs28371706 (CYP2D6*17), rs16947 (CYP2D6*17), rs61736512 (CYP2D6*29), rs1058164 (CYP2D6*29), rs16947 (CYP2D6*29), rs59421388 (CYP2D6*29), rs1135840 (CYP2D6*29), rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023.

In some embodiments, the plurality of genomic loci comprises any 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or all 82 of the genomic loci corresponding to a SNP selected from the group above. Further description of the pharmacogenetic effects, and the therapy recommendations derived therefrom, are provided in the section titled “Single nucleotide polymorphism (SNP) markers,” below.

In some embodiments, these genomic loci and their SNP status may be evaluated in conjunction with other markers known in the art as associated with therapeutic efficacy of treatment of a neuropsychiatric disorder. Exemplary types of the other markers includes but are not limited to gene expression product, epigenetic modification of genomic DNA such as methylation, and metabolic profile/signature. Archer et al., (2010) “Epigenetics and Biomarkers in the Staging of Neuropsychiatric Disorders” Neurotox Res (2010) 18: 347; Quinones et al., “Metabolomics tools for identifying biomarkers for neuropsychiatric diseases,” (2009), Neurobiology of Disease, 35, (2) 165-176.

### IV. Multiplexing for Treatment in Accordance with ICD-10 Codes Associated with Neuropsychiatric Disorders

One aspect of the disclosure provides a method for providing treatment guidance for neuropsychiatric disorder in accordance with ICD-10 codes associated with a neuropsychiatric disorder.

In some embodiments, the ICD-10 codes associated with a neuropsychiatric disorder comprise F31.0, F31.1, F31.2, F31.3, F31.5, F31.6, F31.7, F31.8, F31.9, F32.0, F32.2, F32.3, F32.4, F32.5, F32.8, F32.9, F33.0, F33.1, F33.2, F33.3, F33.4, F33.8, F33.9, F40.0, F40.1, F40.2, F40.8, F40.9, F41.0, F41.1, F41.3, F41.8, F41.9, F42.2, F42.3, F42.4, F42.8, F42.9, F60.5, F90.0, F90.1, F90.2, F90.8, F90.9, F43.1, F84.0, F20.0, F20.1, F20.2, F20.3, F20.5, F20.8, F20.9, F60.0, F60.1, F60.2, F60.3, F60.4, F60.5, F60.6, F60.7, F60.8, F60.9, F07.0, F07.8, F07.9, G89.2, G89.4, F10.1, F10.2, F10.9, F11.1, F11.2, F11.9, F12.1, F12.2, F12.9, F13.1, F13.2, F13.9, F14.1, F14.2, F14.9, F15.1, F15.2, F15.9, F16.1, F16.2, F16.9, F17.2, F18.1, F18.2, F18.9, F19.1, F19.2, F19.9, F55.0, F55.1, F55.2, F55.3, F55.4, or F55.8.

In some embodiments, the neuropsychiatric disorder is major depression, anxiety disorder, obsessive-compulsive disorder, attention deficit hyperactivity disorder (ADHD), bipolar disorder, post-traumatic stress disorder (PTSD), autism, schizophrenia, personality disorder, chronic pain, substance abuse, or any combination thereof.

In some embodiments, the method comprises (A) determining the allele status for a plurality of genomic loci, (B) associating the allele status determined for the plurality of genomic loci with one or more recommendations for the treatment of the neuropsychiatric disorder in accordance with the pertinent ICD-10 codes, and (C) generating a patient-specific report comprising the one or more recommendations for the treatment of the neuropsychiatric disorder. In one non-limiting embodiment, the subject is a human.

In some embodiments, the allele statuses are determined using the multiplex reaction methods described above. For the sake of brevity, a thorough description of these multiplexing methods is not repeated here. Rather, the methods, including each variation described above, is incorporated into this section, in their entireties, as if they were repeated verbatim. The skilled artisan will recognize that the methods above are as equally applicable for the detection of alleles associated with these ICD-10 codes as for any other group of alleles.

A. Analysis of a First Set of Genomic Loci

In one embodiment, a method for providing guidance for the treatment in accordance with an ICD-10 code associated with a neuropsychiatric disorder is provided. The method includes determining the allele status for a plurality of genomic loci, wherein each respective loci in the plurality of loci is associated with a therapeutic efficacy of at least one therapy for a neuropsychiatric disorder. In some embodiments, the determining is performed by amplifying, in a first single in vitro reaction vessel, a first set of two or more genomic loci in the plurality of genomic loci, by polymerase chain reaction (PCR), from nucleic acids isolated from a sample obtained from the subject. As described above, in some embodiments, the first set of genomic loci is at least three loci, or at least four, five, six, seven, eight, nine, ten, or more genomic loci. In some embodiments, as described above, the sample obtained from the subject comprises buccal cells, saliva, or blood.

The amplification is performed in the presence of a plurality of fluorescently-labeled detection reagents, e.g., as described above. The plurality of fluorescently-labeled detection reagents includes, for each respective genomic locus in the two or more genomic loci (i) a first detection reagent that is specific for the presence of a first allele at the respective genomic locus, where the first allele is the most prevalent allele in a population of the species of the subject and the first detection reagent is labeled with a first fluorescent moiety, and (ii) a second detection reagent that is specific for the presence of a second allele at the respective genomic locus, where the second allele is a minor allele in the population and the second detection reagent is labeled with a second fluorescent moiety that is distinguishable from the first fluorescent moiety. As described above, in some embodiments, for each respective genomic locus in the first plurality of genomic loci, the frequency of the first allele in the population is at least 80%, 90%, 95%, or higher. Similarly, as described above, in some embodiments, for each respective genomic locus in the first plurality of genomic loci, the frequency of the second allele in the population is no more than 20%, 10%, 5%, or less.

In some embodiments, the first set of two or more genomic loci include two or more of the genomic loci listed in Table 1 and/or Table 2. In some embodiments, the genomic loci include at least four, five, six, seven, eight, nine, ten, or more genomic loci listed in Table 1 and/or Table 2. Further description of the pharmacogenetic effects, and the therapy recommendations derived therefrom, are provided in the section titled “Single nucleotide polymorphism (SNP) markers,” below.

During or after the amplifying, the method includes detecting a fluorescent signal corresponding to the first fluorescent moiety and the second fluorescent moiety in the first single reaction vessel. Responsive to the detecting, when the contribution of the second fluorescent moiety to the fluorescent signal detected in the reaction vessel does not satisfy a threshold contribution, determining that the subject is homozygous for the first allele at each respective loci in the first set of two or more genomic loci, and when the contribution of the second fluorescent moiety to the fluorescent signal detected in the reaction vessel satisfies the threshold contribution, performing a first plurality of secondary allele detection assays, wherein each secondary allele detection assay in the first plurality of secondary allele detection assays determines the allele status at one respective genomic locus in the first set of two or more genomic loci, thereby determining the allele status at each respective loci in the first set of two or more genomic loci.

In some embodiments, the method then includes associating the allele status determined for the plurality of genomic loci with one or more recommendations for the treatment of a neuropsychiatric disorder. In some embodiments, this is performed by a suitably programed computer, e.g., having a look-up table with known pharmacogenetic associations for each variant allele detectable by the assay. Example systems for performing this step are described in more detail below.

In some embodiments, the method then includes generating a patient-specific report including one or more recommendations for the treatment of the neuropsychiatric disorder in fulfillment of an ICD-10 code selected from F31.0, F31.1, F31.2, F31.3, F31.5, F31.6, F31.7, F31.8, F31.9, F32.0, F32.2, F32.3, F32.4, F32.5, F32.8, F32.9, F33.0, F33.1, F33.2, F33.3, F33.4, F33.8, F33.9, F40.0, F40.1, F40.2, F40.8, F40.9, F41.0, F41.1, F41.3, F41.8, F41.9, F42.2, F42.3, F42.4, F42.8, F42.9, F60.5, F90.0, F90.1, F90.2, F90.8, F90.9, F43.1, F84.0, F20.0, F20.1, F20.2, F20.3, F20.5, F20.8, F20.9, F60.0, F60.1, F60.2, F60.3, F60.4, F60.5, F60.6, F60.7, F60.8, F60.9, F07.0, F07.8, F07.9, G89.2, G89.4, F10.1, F10.2, F10.9, F11.1, F11.2, F11.9, F12.1, F12.2, F12.9, F13.1, F13.2, F13.9, F14.1, F14.2, F14.9, F15.1, F15.2, F15.9, F16.1, F16.2, F16.9, F17.2, F18.1, F18.2, F18.9, F19.1, F19.2, F19.9, F55.0, F55.1, F55.2, F55.3, F55.4, and F55.8. In some embodiments, this is again performed by a suitably programed computer. Example systems for performing this step are described in more detail below.

B. Analysis of a Second Set of Genomic Loci

In some embodiments, a second set of loci can be tested in a similar fashion as the first set of loci, using a second reaction vessel. In such fashion, an even larger number of loci can be tested at a single time.

Accordingly, in some embodiments of the amplifying step described above, a second set of two or more genomic loci in the plurality of genomic loci are amplified, in a second single in vitro reaction vessel, by polymerase chain reaction (PCR), from nucleic acids isolated from a sample obtained from the subject, in the presence of a plurality of fluorescently-labeled detection reagents. The plurality of fluorescently-labeled detection reagents includes, for each respective genomic locus in the second set of two or more genomic loci, (i) a third detection reagent that is specific for the presence of a first allele at the respective genomic locus, where the first allele is the most prevalent allele in a population of the species of the subject and the third detection reagent is labeled with a third fluorescent moiety, and (ii) a fourth detection reagent that is specific for the presence of a second allele at the respective genomic locus, wherein the second allele is a minor allele in the population and the fourth detection reagent is labeled with a fourth fluorescent moiety that is distinguishable from the third fluorescent moiety.

During or after the amplifying, the method includes detecting a fluorescent signal corresponding to the third fluorescent moiety and the fourth fluorescent moiety in the second single reaction vessel. In some embodiments, the third fluorescent moiety and the fourth fluorescent moiety are the same as the first fluorescent moiety and the second fluorescent moiety. This is possible because the first plurality of loci and the second plurality of loci are assayed in different reaction vessels. In some embodiments, the third fluorescent moiety and the fourth fluorescent moiety are different than the first fluorescent moiety and the second fluorescent moiety.

Likewise, in some embodiments of the reporting step described above, responsive to the detecting, (i) when the contribution of the fourth fluorescent moiety to the fluorescent signal detected in the reaction vessel does not satisfy a threshold contribution, determining that the subject is homozygous for the first allele at each respective loci in the second set of two or more loci, and (ii) when the contribution of the fourth fluorescent moiety to the fluorescent signal detected in the reaction vessel satisfies the threshold contribution, performing a second plurality of secondary allele detection assays, where each secondary allele detection assay in the second plurality of secondary allele detection assays determines the allele status at one respective genomic locus in the second set of two or more genomic loci, thereby determining the allele status at each respective loci in the second set of two or more genomic loci.

C. Analysis of a Third Set of Genomic Loci

In some embodiments, a third set of loci can be tested in a similar fashion as the first set of loci and second set of loci, using a third reaction vessel. In such fashion, an even larger number of loci can be tested at a single time.

Accordingly, in some embodiments of the amplifying step described above, a third set of two or more genomic loci in the plurality of genomic loci are amplified, in a third single in vitro reaction vessel, by polymerase chain reaction (PCR), from nucleic acids isolated from a sample obtained from the subject, in the presence of a plurality of fluorescently-labeled detection reagents. The plurality of fluorescently-labeled detection reagents includes, for each respective genomic locus in the third set of two or more genomic loci, (i) a fifth detection reagent that is specific for the presence of a first allele at the respective genomic locus, where the first allele is the most prevalent allele in a population of the species of the subject and the fifth detection reagent is labeled with a fifth fluorescent moiety, and (ii) a sixth detection reagent that is specific for the presence of a second allele at the respective genomic locus, wherein the second allele is a minor allele in the population and the sixth detection reagent is labeled with a sixth fluorescent moiety that is distinguishable from the fifth fluorescent moiety.

During or after the amplifying, the method includes detecting a fluorescent signal corresponding to the fifth fluorescent moiety and the sixth fluorescent moiety in the second single reaction vessel. In some embodiments, the fifth fluorescent moiety and the sixth fluorescent moiety are the same as the first fluorescent moiety and the second fluorescent moiety. This is possible because the first plurality of loci and the third plurality of loci are assayed in different reaction vessels. In some embodiments, the fifth fluorescent moiety and the sixth fluorescent moiety are different from the first fluorescent moiety and the second fluorescent moiety.

Likewise, in some embodiments of the reporting step described above, responsive to the detecting, (i) when the contribution of the sixth fluorescent moiety to the fluorescent signal detected in the reaction vessel does not satisfy a threshold contribution, determining that the subject is homozygous for the first allele at each respective loci in the third set of two or more loci, and (ii) when the contribution of the sixth fluorescent moiety to the fluorescent signal detected in the reaction vessel satisfies the threshold contribution, performing a third plurality of secondary allele detection assays, where each secondary allele detection assay in the third plurality of secondary allele detection assays determines the allele status at one respective genomic locus in the third set of two or more genomic loci, thereby determining the allele status at each respective loci in the third set of two or more genomic loci.

D. Genomic Loci Associated with Neuropsychiatric Disorders

In some embodiments, the plurality of genomic loci comprises one or more genomic loci corresponding to a SNP selected from the group consisting of rs7997012, rs3813929, rs1045642, rs2032583, rs1800544, rs10994336, rs6265, rs1006737, rs4680, rs2470890 (CYP1A2*1B), rs2069514, rs35694136, rs2069526 (CYP1A2*1E), rs762551, rs12720461 (CYP1A2*1K), rs2069526 (CYP1A2*1K), rs72547513, rs2279343 (CYP2B6*4), rs3211371, rs3745274 (CYP2B6*6), rs2279343 (CYP2B6*6), rs4244285, rs17878459 (CYP2C19*2B), rs4986893 (CYP2C19*3), rs57081121 (CYP2C19*3), rs28399504, rs56337013, rs72552267, rs72558186, rs41291556, rs17884712, rs6413438, rs12248560, rs12769205 (CYP2C19*35), rs3758581 (CYP2C19*35), rs1799853, rs1057910, rs56165452, rs28371686, rs9332131, rs7900194, rs28371685, rs72558187, rs7900194 (CYP2C9*27), rs16947 (CYP2D6*2), rs1135840 (CYP2D6*2), rs1135824 (CYP2D6*3), rs35742686 (CYP2D6*3), rs3892097, rs5030655, rs5030867, rs5030865, rs5030656, rs1065852, rs5030863, rs5030862, rs5030865, rs774671100, rs28371706 (CYP2D6*17), rs16947 (CYP2D6*17), rs61736512 (CYP2D6*29), rs1058164 (CYP2D6*29), rs16947 (CYP2D6*29), rs59421388 (CYP2D6*29), rs1135840 (CYP2D6*29), rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023.

In some embodiments, the plurality of genomic loci comprises at least the genomic loci corresponding to SNPs rs7997012, rs3813929, rs1045642, rs2032583, rs1800544, rs10994336, rs6265, rs1006737, rs4680, rs2470890 (CYP1A2*1B), rs2069514, rs35694136, rs2069526 (CYP1A2*1E), rs762551, rs12720461 (CYP1A2*1K), rs2069526 (CYP1A2*1K), rs72547513, rs2279343 (CYP2B6*4), rs3211371, rs3745274 (CYP2B6*6), rs2279343 (CYP2B6*6), rs4244285, rs17878459 (CYP2C19*2B), rs4986893 (CYP2C19*3), rs57081121 (CYP2C19*3), rs28399504, rs56337013, rs72552267, rs72558186, rs41291556, rs17884712, rs6413438, rs12248560, rs12769205 (CYP2C19*35), rs3758581 (CYP2C19*35), rs1799853, rs1057910, rs56165452, rs28371686, rs9332131, rs7900194, rs28371685, rs72558187, rs7900194 (CYP2C9*27), rs16947 (CYP2D6*2), rs1135840 (CYP2D6*2), rs1135824 (CYP2D6*3), rs35742686 (CYP2D6*3), rs3892097, rs5030655, rs5030867, rs5030865, rs5030656, rs1065852, rs5030863, rs5030862, rs5030865, rs774671100, rs28371706 (CYP2D6*17), rs16947 (CYP2D6*17), rs61736512 (CYP2D6*29), rs1058164 (CYP2D6*29), rs16947 (CYP2D6*29), rs59421388 (CYP2D6*29), rs1135840 (CYP2D6*29), rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023.

In some embodiments, the plurality of genomic loci comprises any 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82 of the genomic loci corresponding to a SNP selected from the group. Further description of the pharmacogenetic effects, and the therapy recommendations derived therefrom, are provided in the section titled “Single nucleotide polymorphism (SNP) markers,” below.

In some embodiments, these genomic loci and their SNP status may be evaluated in conjunction with other markers known in the art as associated with therapeutic efficacy of treatment of a neuropsychiatric disorder. Exemplary types of the other markers includes but are not limited to gene expression product, epigenetic modification of genomic DNA such as methylation, and metabolic profile/signature. Archer et al., (2010) “Epigenetics and Biomarkers in the Staging of Neuropsychiatric Disorders” Neurotox Res (2010) 18: 347; Quinones et al., “Metabolomics tools for identifying biomarkers for neuropsychiatric diseases,” (2009), Neurobiology of Disease, 35, (2) 165-176.

E. Post-Determination of Allele Status

In some embodiments, the method further comprises (B) associating the allele status determined for the plurality of genomic loci in a subject with one or more recommendations for the treatment of a condition within/in fulfillment of an ICD-10 code.

In some embodiments, the ICD-10 code is selected from the group consisting of F31.0, F31.1, F31.2, F31.3, F31.5, F31.6, F31.7, F31.8, F31.9, F32.0, F32.2, F32.3, F32.4, F32.5, F32.8, F32.9, F33.0, F33.1, F33.2, F33.3, F33.4, F33.8, F33.9, F40.0, F40.1, F40.2, F40.8, F40.9, F41.0, F41.1, F41.3, F41.8, F41.9, F42.2, F42.3, F42.4, F42.8, F42.9, F60.5, F90.0, F90.1, F90.2, F90.8, F90.9, F43.1, F84.0, F20.0, F20.1, F20.2, F20.3, F20.5, F20.8, F20.9, F60.0, F60.1, F60.2, F60.3, F60.4, F60.5, F60.6, F60.7, F60.8, F60.9, F07.0, F07.8, F07.9, G89.2, G89.4, F10.1, F10.2, F10.9, F11.1, F11.2, F11.9, F12.1, F12.2, F12.9, F13.1, F13.2, F13.9, F14.1, F14.2, F14.9, F15.1, F15.2, F15.9, F16.1, F16.2, F16.9, F17.2, F18.1, F18.2, F18.9, F19.1, F19.2, F19.9, F55.0, F55.1, F55.2, F55.3, F55.4, or F55.8.

In some embodiments, the method further comprises (C) generating a patient-specific report comprising the one or more recommendations for the treatment of a condition within/in fulfillment of the ICD-10 code. The detailed method for generating a patient-specific report is provided in subsequent sections.

In one embodiment, the treatment guidance is for bipolar disorder and the ICD-10 code is F31.0 (bipolar disorder, current episode hypomanic), F31.1 (bipolar disorder, current episode manic without psychotic features), F31.2 (bipolar disorder, current episode manic severe with psychotic features), F31.3 (bipolar disorder, current episode depressed, mild or moderate severity), F31.5 (bipolar disorder, current episode depressed, severe, with psychotic features), F31.6 (bipolar disorder, current episode mixed), F31.7 (bipolar disorder, currently in remission), F31.8 (other bipolar disorders), or F31.9 (bipolar disorder, unspecified).

In another embodiment, the treatment guidance is for major depressive disorder, single episode and the ICD-10 code is F32.0 (major depressive disorder, single episode, mild), F32.1 (major depressive disorder, single episode, moderate), F32.2 (major depressive disorder, single episode, severe without psychotic features), F32.3 (major depressive disorder, single episode, severe with psychotic features), F32.4 (major depressive disorder, single episode, in partial remission), F32.5 (major depressive disorder, single episode, in full remission), F32.8 (other depressive episodes), F32.9 (major depressive disorder, single episode, unspecified), F33.0 (major depressive disorder, recurrent, mild), F33.1 (major depressive disorder, recurrent, moderate), F33.2 (major depressive disorder, recurrent severe without psychotic features), F33.3 (major depressive disorder, recurrent, severe with psychotic symptoms), F33.4 (major depressive disorder, recurrent, in remission), F32.4 (major depressive disorder, single episode, in partial remission), F33.8 (other recurrent depressive disorders), or F33.9 (major depressive disorder, recurrent, unspecified).

In yet another embodiment, the treatment guidance is for anxiety disorder and the ICD-10 code is F40.0 (agoraphobia), F40.1 (social phobias), F40.2 (specific (isolated) phobias), F40.8 (other phobic anxiety disorders), F40.9 (phobic anxiety disorder, unspecified), F41.0 (panic disorder (episodic paroxysmal anxiety)), F41.1 (generalized anxiety disorder), F41.3 (other mixed anxiety disorders), F41.8 (other specified anxiety disorders), or F41.9 (anxiety disorder, unspecified).

In yet another embodiment, the treatment guidance is for obsessive-compulsive disorder and the ICD-10 code is F42.2 (mixed obsessional thoughts and acts), F42.3 (hoarding disorder), F42.4 (excoriation (skin-picking) disorder), F42.8 (other obsessive-compulsive disorder), F42.9 (obsessive-compulsive disorder, unspecified), or F60.5 (obsessive-compulsive personality disorder).

In yet another embodiment, the treatment guidance is for attention-deficit hyperactivity disorder and the ICD-10 code is F90.0 (attention-deficit hyperactivity disorder, predominantly inattentive type), F90.1 (attention-deficit hyperactivity disorder, predominantly hyperactive type), F90.2 (attention-deficit hyperactivity disorder, combined type), F90.8 (attention-deficit hyperactivity disorder, other type), or F90.9 (attention-deficit hyperactivity disorder, unspecified type).

In yet another embodiment, the treatment guidance is for post-traumatic stress disorder and the ICD-10 code is F43.1 (post-traumatic stress disorder (PTSD)).

In yet another embodiment, the treatment guidance is for autistic disorder and the ICD-10 code is F84.0 (autistic disorder).

In yet another embodiment, the treatment guidance is for schizophrenia and the ICD-10 code is F20.0 (paranoid schizophrenia), F20.1 (disorganized schizophrenia) F20.2 (catatonic schizophrenia) F20.3 (undifferentiated schizophrenia) F20.5 (residual schizophrenia), F20.8 (other schizophrenia), or F20.9 (schizophrenia, unspecified).

In yet another embodiment, the treatment guidance is for personality disorder and the ICD-10 code is F60.0 (paranoid personality disorder), F60.1 (schizoid personality disorder), F60.2 (antisocial personality disorder), F60.3 (borderline personality disorder), F60.4 (histrionic personality disorder), F60.5 (obsessive-compulsive personality disorder), F60.6 (avoidant personality disorder), F60.7 (dependent personality disorder), F60.8 (other specific personality disorders), F60.9 (personality disorder, unspecified), F07.0 (personality change due to known physiological condition), F07.8 (other personality and behavioral disorders due to known physiological condition), or F07.9 (unspecified personality and behavioral disorder due to known physiological condition).

In yet another embodiment, the treatment guidance is for chronic pain and the ICD-10 code is G89.2 (chronic pain, not elsewhere classified), or G89.4 (chronic pain syndrome).

In yet another embodiment, the treatment guidance is for substance abuse and the ICD-10 code is F10.1 (alcohol abuse), F10.2 (alcohol dependence), F10.9 (alcohol use, unspecified), F11.1 (opioid abuse), F11.2 (opioid dependence), F11.9 (opioid use, unspecified), F12.1 (cannabis abuse), F12.2 (cannabis dependence), F12.9 (cannabis use, unspecified), F13.1 (sedative, hypnotic or anxiolytic-related abuse), F13.2 (sedative, hypnotic or anxiolytic-related dependence), F13.9 (sedative, hypnotic or anxiolytic-related use, unspecified), F14.1 (cocaine abuse), F14.2 (cocaine dependence), F14.9 (cocaine use, unspecified), F15.1 (other stimulant abuse), F15.2 (other stimulant dependence), F15.9 (other stimulant use, unspecified), F16.1 (hallucinogen abuse), F16.2 (hallucinogen dependence), F16.9 (hallucinogen use, unspecified), F17.2 (nicotine dependence), F18.1 (inhalant abuse), F18.2 (inhalant dependence), F18.9 (inhalant use, unspecified), F19.1 (other psychoactive substance abuse), F19.2 (other psychoactive substance dependence), F19.9 (other psychoactive substance use, unspecified), F55.0 (abuse of antacids), F55.1 (abuse of herbal or folk remedies), F55.2 (abuse of laxatives), F55.3 (abuse of steroids or hormones), F55.4 (abuse of vitamins), or F55.8 (abuse of other non-psychoactive substances).

### V. TaqMan® Genotyping Assay

As is widely known to those of ordinary sill in the art, the TaqMan® assay is performed concurrently with PCR and the results can be read in real-time as the PCR proceeds. The assay uses forward and reverse PCR primers that amplify a nucleic acid sequence that encompasses the genomic loci corresponding to a SNP or variants thereof.

TaqMan® assay is a common PCR amplification-based technique for characterizing allelic status of genomic loci. In a conventional, singleplex TaqMan® assay, allele status determination can be achieved using EET (electronic energy transfer)/FRET (fluorescence resonance energy transfer) signals combined with one or two allele-specific probes (i.e., detection reagents).

Probes for TaqMan® assays are designed such that they anneal within a DNA region amplified by a specific set of primers. The probes generally used have an excitation chromophore linked to their 5′ end and a quencher chromophore linked to their 3′ end. While the probe is intact, as is the case when the probe is isolated/unbound to any complementary nucleic acids, the quencher will remain in close proximity to the excitation chromophore, eliminating the chromophore's fluorescence. During the PCR amplification step, allele-specific probes and its non-specific counterpart probes compete for binding to the corresponding allele. If the allele-specific probe is perfectly complementary to the SNP allele, it will bind to the target DNA strand and then get cleaved by 5′-nuclease activity of the Taq polymerase as it extends the DNA from the PCR primers. The cleavage of the probe results in spatial separation of the excitation chromophore from the quencher molecule, generating a detectable signal due to the excitation chromophore's fluorescence. If the allele-specific probe is not perfectly complementary, it will have lower melting temperature and not bind as efficiently. As such, the non-specific probe will be outcompeted by the allele-specific probe in binding to the allele, where the binding between the allele and the probe will be saturated by the allele-specific probe. This prevents the nuclease from acting on the non-specific probe.

Normally, when more than one excitation chromophores are used to label various probes in a single reaction vessel, the emission spectra of the various excitation chromophores used in the same reaction tube should differ from one another such that the emitted fluorescence originating from each excitation chromophore can be distinguished by the real-time PCR apparatus capable of detecting fluorescent signal. Yet in other cases, the emission spectra of excitation chromophores may overlap to the extent that detection of fluorescent signal from various excitation chromophores can be combined.

A. PCR Primers

PCR primers are important components for the amplification of a template nucleic acid sequence using PCR. Primers are synthetic oligonucleotides of approximately 15-30 nucleotides. PCR primers are designed to bind (i.e., anneal) to sequences that flank the region of interest in the template DNA that are complementary in nucleotide sequences. During PCR, enzymatic DNA polymerase extends the primers from their 3′ ends to their 5′ ends such that a new copy of the template DNA sequence can be formed in the end of the extension process. As such, the primers' annealing sites must be unique to the vicinity of the target with minimal crossover with other regions of the template DNA to ensure specific amplification of the intended target.

Additionally, primers design must consider other factors to ensure specificity in amplification. First, primer sequences should possess melting temperatures (Tm) in the range of 55-70° C., with the Tms of the two primers within 5° C. of each other. Second, the primers should be designed without complementarity between the primers (especially at their 3′ ends) that causes their annealing (i.e., primer-dimers), self-complementarity that can cause self-priming (i.e., secondary structures), or direct repeats that can create imperfect alignment with the target area of the template.

Furthermore, the guanine-cytosine content (GC-content) of the primer should ideally be 40-60%, with minimal repetition of cytosine (C) and guanine (G) nucleotides and uniform distribution of C and G bases. No more than three G or C bases should be present at the 3′-ends of the primers. Moreover, one C or G nucleotide at the 3′ end of a primer can enhance specific binding and extension of a primer.

Primers with long sequences such as more than 50 nucleotide bases frequently requires to be purified to remove byproducts and unconjugated nucleotides from the chemical synthesis process.

When designing primers for PCR cloning, non-template sequences such as restriction sites, recombination sequences, and promoter binding sites can be introduced to the 5′ ends as extensions. These extension sequences need to be carefully designed for minimal impact on PCR amplification and downstream applications.

In addition, primer concentration needs to be optimized for PCR. In PCR setup, primers are added to the reaction in the concentration range of 0.1-1 μM. For primers with degenerate bases or those used in long PCR, primer concentrations of 0.3-1 μM are often recommended. A general recommendation is to start with standard concentrations and adjust as necessary. Higher primer concentrations often contribute to non-specific binding and amplification of unwanted sequences. On the other hand, low primer concentrations can result in low or no yield of the desired target.

Lastly, methods of designing PCR primers are well-known to a person of ordinary skill in the art. guidelines and protocols for the methods are available, including NCBI Primer-Blast at https://www.ncbi.nlm.nih.gov/tools/primer-blast/, Innis et al. (1990) PCR Protocols, A Guide to Methods and Applications, Academic Press, Inc. N.Y.), and Dieffenbach et al. General Concept for PCR Primer Design, Genome Res. (1993). 3: S30-S37.

B. Probes

1. General Principle of Electronic Energy Transfer and Quenching

Electronic energy transfer (EET), fluorescence resonance energy transfer (FRET), resonance energy transfer (RET), or Förster resonance energy transfer (FRET), is a mechanism describing energy transfer between two chromophores—an excitation chromophore and a acceptor/quencher. An excitation chromophore, also often referred to as donor or donor chromophore, initially in its electronic excited state, may transfer energy to an acceptor chromophore, through non-radiative dipole-dipole coupling. A quencher is a chromophore of which the absorption spectrum overlaps with the emission spectrum of a pairing excitation chromophore. The overall process of excitation, transfer to a second chromophore is called electronic energy transfer (EET) or fluorescence resonance energy transfer (FRET).

In particular, when an excitation chromophore is excited at a particular wavelength, it is then promoted to an excited state. In the absence of a quencher, the excited chromophore emits light in returning to the ground state. If a quencher chromophore is in the vicinity of an excitation chromophore, then the excited chromophore can return to the ground state by transferring its energy to the quencher, without the emission of light, while the quencher is promoted to its excited state. The excited quencher will later return to the ground state via non-radiative decay pathways, without the emission of light. The As such, EET/FRET depends on the ability of the excitation chromophore to transfer energy to the quencher. The efficiency of the energy transfer between an excitation chromophore and an quencher chromophore is inversely proportional to the sixth power of the distance between the two chromophores, which makes FRET highly sensitive to small changes in distance on the level of nanometers. Thus, measurements of FRET efficiency can be used to determine if (donor) chromophore and acceptor are within a certain distance from each other, typically in the range of 1-10 nm.

As a powerful tool in molecular biology, EET is frequently used to characterize molecular dynamics in biophysics and biochemistry, especially molecular interaction such as DNA-DNA interactions, DNA-protein interactions, and protein-protein interactions. Oftentimes, various molecules or components within a molecule are labeled chromophores to monitor intermolecular formation of a complex or intra-molecular conformational changes, as reflected by changes in distance between the excitation and the acceptor chromophores.

EET in TaqMan® assays relies on the utilization of double-labeled TaqMan® probes, which are disclosed in the U.S. Pat. Nos. 5,210,015 and 5,487,972. Double-labeled TaqMan® probes carry two chromophores on a probe comprising an oligonucleotide sequence that hybridizes to a target nucleic acid. The excitation chromophores is here located at the 5′ end, the quencher chromophore at the 3′ end. In addition, there may still be a phosphate group on the 3-end of the probe, so that the probe cannot function as a primer during PCR amplification. As long as the probe is intact, the intensity of light emission is minimal, because almost all of the light energy produced by the excitation of the excitation chromophore is absorbed and transformed by the quencher due to its proximity. As such, the emitted light from the excitation chromophore is “quenched.” This EET effect is also retained after the probe has bound to the complementary DNA strand. However, during PCR amplification, as a primer extends themselves along the template DNA strand from its 5′ end to the 3′ end, polymerases with exonuclease activity that travels along the primer hits the probe and hydrolyzes it, thereby setting free the 5′ excitation chromophore from the vicinity of the quencher chromophore into solution. As more and more DNAs are amplified during PCR, this eventually leads to an increase in fluorescent signal corresponding to the excitation chromophores that can be registered and measured.

2. Selection of Excitation and Quencher Chromophores

Some of the well-known commercially available excitation chromophores for EET techniques include but are not limited to: 5- or 6-carboxyfluorescein (FAM™), VIC™, NED™, fluorescein, fluorescein isothiocyanate (FITC), IRDYE-700/800, cyanine dyes, such as CY3™, CY5™, CY3.5™, CY5.5™, Cy7™, xanthen, 6-carboxy-2′,4′,7′,4,7-hexachlorofluorescein (HEX), 6-carboxy-1,4-dichloro-2′,7′-dichloro-fluorescein (TET®), 6-carboxy-4′,5′-dichloro-2′,7′-dimethodyfluorescein (JOE™), N,N,N′,N′-tetramethyl-6-carboxyrhodamine (TAMRA™), 6-carboxy-X-rhodamine (ROX), 5-carboxyrhodamine-6G (R6G5), 6-carboxyrhodamine-6G (RG6), rhodamine, rhodamine green, rhodamine red, rhodamine 110, Rhodamin 6G®, BODIPY dyes, such as BODIPY TMR, oregon green, coumarines, such as umbelliferone, benzimides, such as Hoechst 33258; phenanthridines, such as Texas Red®, California Red®, Yakima Yellow, Alexa Fluor® 350, Alexa Fluor® 405, Alexa Fluor® 430, Alexa Fluor® 488, Alexa Fluor® 500, Alexa Fluor® 514, Alexa Fluor0532, Alexa Fluor® 546, Alexa Fluor® 555, Alexa Fluor® 568, Alexa Fluor® 594, Alexa Fluor® 610, Alexa Fluor® 633, Alexa Fluor® 647, Alexa Fluor® 660, Alexa Fluor® 680, Alexa Fluor® 700, Alexa Fluor® 750, PET®, ethidium bromide, acridinium dyes, carbazol dyes, phenoxazine dyes, porphyrine dyes, polymethin dyes, Atto 390, Atto 425, Atto 465, Atto 488, Atto 495, Atto 520, Atto 532, Atto 550, Atto 565, Atto 590, Atto 594, Atto 620, Atto 633, Atto 647N, Atto 655, Atto RhoG6, Atto Rhol1, Atto Rhol2, Atto Rhol01, BMN™-5, BMN™-6, CEQ8000 D2, CEQ8000 D3, CEQ8000 D4, DY-480XL, DY-485XL, DY-495, DY-505, DY-510XL, DY-521XL, DY-521XL, DY-530, DY-547, DY-550, DY-555, DY-610, DY-615, DY-630, DY-631, DY-633, DY-635, DY-647, DY-651, DY-675, DY-676, DY-680, DY-681, DY-700, DY-701, DY-730, DY-731, DY-732, DY-750, DY-751, DY-776, DY-780, DY-781, DY-782, 6-carboxy-4′,5′-dichloro-2′,7′-dimethoxyfluorescein (JOE), TET™, CAL Fluor® Gold 540, CAL Fluor RED 590, CAL Fluor Red 610, CAL Fluor Red 635, IRDye® 700Dx, IRDye® 800CW, Marina Blue®, Pacific Blue®, Yakima Yellow®, 6-(4,7-Dichloro-2′,7′-diphenyl-3′,6′-dipivaloylfluorescein-6-carboxamido)-hexyl-1-O-(2-cyanoethyl)-(N,N-diisopropyl)-phosphoramidite (SIMA), CAL Fluor® Gold 540, CAL Fluor® Orange 560, CAL Fluor Red 635, Quasar 570, Quasar 670, LIZ, Sunnyvale Red, LC Red® 610, LC Red® 640, LC Red® 670, and LC Red® 705.

Well-known commercially available non-fluorescent quenchers include but not are limited to, Black Hole Quencher® (BHQ®), DQ, dimethylaminoazobenzenesulfonic acid (DABCYL), Iowa Black® (IWB), and TAMRA. Further discussion of these molecules is provided by Johansson, M. K, et al, J. Am. Chem., Soc., (2002). These and similar non-fluorescent quenchers improve the sensitivity of TaqMan® probes by suppressing background fluorescence, thereby increasing the signal gain following enzymatic cleavage of the probe.

As discussed above, it is known to those of skill in the art that, for ETT to occur, the emission spectrum of the excitation chromophore and the absorption spectrum of the quencher chromophore typically need to overlap. And such a person is able to ascertain suitable combinations of an excitation chromophore and a quencher. Examples of some of the commonly used paring chromophores for EET-based techniques include but are not limited to: FAM/TAMRA, VIC/BHQ1, HEX/BHQ2, Cy3/BHQ1, Cy5/BHQ1, and TET/DHQ and the like. Examples of guidelines and protocols of selecting suitable chromophores combinations and making necessary adjustments include Lee et al. (1997), “New energy transfer dyes for DNA sequencing,” Nucleic Acids Research 25:2816; Bajar, Bryce T et al. (2016), “A Guide to Fluorescent Protein FRET Pairs,” Sensors (Basel, Switzerland) vol. 16, 9 1488. It will be readily apparent to those of ordinary skill in the art in light of the teachings herein that certain changes and modifications may be made thereto without departing from the spirit and scope of the disclosure.

3. Length of Oligonucleotide TaqMan® Probes

In addition to the spectral overlap requirement stated above, a second parameter critical to EET-based TaqMan® assay relates to the length of oligonucleotide portion of a TaqMan® probe, i.e., the number of nucleotide bases in the oligonucleotide probe. In some cases, the oligonucleotide portion of a TaqMan® probe has from 8 to 18 nucleotide bases. In some other cases, the probe has a length of from 10 to 18 bases. See U.S. Pat. No. 7,667,024. In some cases, the probes are 5-100 nucleotides in length, more preferably between 5-25 nucleotides in length, and even more preferably 5-12 nucleotides in length. In yet other cases, the length may be at least 5 nucleotides, at least 10 nucleotides, at least 11 nucleotides, at least 12 nucleotides, at least 13 nucleotides, at least 14 nucleotides, at least 15 nucleotides, at least 20 nucleotides, or at least 25 nucleotides, or more, in length. See PCT Publication No. WO 2007/002375. In yet another case, a probe has a length of about 32 to about 35 nucleotides (Keohler et al. (2005), “Effects of DNA secondary structure on oligonucleotide probe binding efficiency,” Comput Biol. Chem. 29(6):393-7; and Keohler et al. (2005) “Thermodynamic properties of DNA sequences: characteristic values for the human genome,” Bioinformatics 21(16):3333-9.)

The size consideration of TaqMan® probes is critical. EET is extremely sensitive to the relative distance change between matching chromophores, since the energy transfer is dependent on the inverse sixth power of the intermolecular separation. As such, the quenching effect in uncleaved/unhydrolyzed fluorogenic probes is largely dependent on the size of the oligonucleotide separating the excitation chromophore and the quencher.

In addition, the length of the probe can determine the specificity of the binding of a probe to its target nucleic acid sequence. The energetic cost of a mismatch between the probe and its target is relatively higher for shorter sequences than for longer ones. Therefore, hybridization of smaller nucleic acid probes is more specific than is hybridization of longer nucleic acid probes to the same target because the longer probes can tolerate more mismatches yet still continue to bind to the target. Zhang et al., (2007), “Free energy of DNA duplex formation on short oligonucleotide microarrays”, Nucleic Acids Research, Volume 35, Issue 3.

Also relevant to the size consideration here is the composition of other chemical conjugates to a TaqMan® probe. Chemical conjugates may affect the binding affinity of a probe, thereby affecting the length requirement for said probes. One non-limiting example of the chemical conjugate for oligonucleotide probes is minor groove binder (MGB), a molecule that binds within the minor groove of double stranded DNA. Oligonucleotide probes with conjugated MGB groups can bind to target nucleic acid sequences more efficiently, thereby allowing probes shorter than typical TaqMan® probes to be used for genotyping assays. Kutyavin et al., (2000), “3′-Minor groove binder-DNA probes increase sequence specificity at PCR extension temperatures,” Nucleic Acids Research, Volume 28, Issue 2, Pages 655-661. A variety of suitable minor groove binders have been described in the literature. See, for example, Kutyavin, et al. U.S. Pat. No. 5,801,155; Wemmer, D. E., and Dervan P. B., Current Opinion in Structural Biology, 7:355-361 (1997); Walker, W. L, Kopka, J. L. and Goodsell, D. S., Biopolymers, 44:323-334 (1997); Zimmer, C.& Wahnert, U. Prog. Biophys. Molec. Bio. 47:31-112 (1986) and Reddy, B. S. P., Dondhi, S. M., and Lown, J. W., Pharmacol. Therap., 84:1-111 (1999) (the disclosures of which are herein incorporated by reference in their entireties). A commonly used MGB is DPI3.

Thus, the length of a TaqMan® probe must be carefully determined when designing the probe. A person of ordinary skill in the art would be able to ascertain the optimal length and appreciate the need to weigh the various factors above and fine-tune the length of TaqMan® probes, such that an intact, unhydrolyzed TaqMan® probe does not emit significant amount of light.

### VI. Single Nucleotide Polymorphism (SNP) Markers

The present disclosure provides methods for determining the allelic status of multiple genomic loci in a subject by using allele-specific detection reagents/probes in a multiplex fashion. In one embodiment, the method provided herein includes screening across a plurality of genomic loci in one or more test subjects/patients for the presence of SNP markers listed in Table 1 and/or Table 2. The results obtained in the genotyping assay may then be apply to guiding or modifying a course of therapy in an individual patient, including generating a patient-specific report comprising treatment recommendations for treating a neuropsychiatric disorder, in accordance with the detected SNPs of the patient.

In some embodiments, the plurality of genomic loci associated with a neuropsychiatric disorder includes one or more genomic loci corresponding to a SNP selected from the group consisting of rs7997012, rs3813929, rs1045642, rs2032583, rs1800544, rs10994336, rs6265, rs1006737, rs4680, rs2470890 (CYP1A2*1B), rs2069514, rs35694136, rs2069526 (CYP1A2*1E), rs762551, rs12720461 (CYP1A2*1K), rs2069526 (CYP1A2*1K), rs72547513, rs2279343 (CYP2B6*4), rs3211371, rs3745274 (CYP2B6*6), rs2279343 (CYP2B6*6), rs4244285, rs17878459 (CYP2C19*2B), rs4986893 (CYP2C19*3), rs57081121 (CYP2C19*3), rs28399504, rs56337013, rs72552267, rs72558186, rs41291556, rs17884712, rs6413438, rs12248560, rs12769205 (CYP2C19*35), rs3758581 (CYP2C19*35), rs1799853, rs1057910, rs56165452, rs28371686, rs9332131, rs7900194, rs28371685, rs72558187, rs7900194 (CYP2C9*27), rs16947 (CYP2D6*2), rs1135840 (CYP2D6*2), rs1135824 (CYP2D6*3), rs35742686 (CYP2D6*3), rs3892097, rs5030655, rs5030867, rs5030865, rs5030656, rs1065852, rs5030863, rs5030862, rs5030865, rs774671100, rs28371706 (CYP2D6*17), rs16947 (CYP2D6*17), rs61736512 (CYP2D6*29), rs1058164 (CYP2D6*29), rs16947 (CYP2D6*29), rs59421388 (CYP2D6*29), rs1135840 (CYP2D6*29), rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023.

In some embodiments, the plurality of genomic loci associated with a neuropsychiatric disorder includes at least the genomic loci corresponding to SNPs rs7997012, rs3813929, rs1045642, rs2032583, rs1800544, rs10994336, rs6265, rs1006737, rs4680, rs2470890 (CYP1A2*1B), rs2069514, rs35694136, rs2069526 (CYP1A2*1E), rs762551, rs12720461 (CYP1A2*1K), rs2069526 (CYP1A2*1K), rs72547513, rs2279343 (CYP2B6*4), rs3211371, rs3745274 (CYP2B6*6), rs2279343 (CYP2B6*6), rs4244285, rs17878459 (CYP2C19*2B), rs4986893 (CYP2C19*3), rs57081121 (CYP2C19*3), rs28399504, rs56337013, rs72552267, rs72558186, rs41291556, rs17884712, rs6413438, rs12248560, rs12769205 (CYP2C19*35), rs3758581 (CYP2C19*35), rs1799853, rs1057910, rs56165452, rs28371686, rs9332131, rs7900194, rs28371685, rs72558187, rs7900194 (CYP2C9*27), rs16947 (CYP2D6*2), rs1135840 (CYP2D6*2), rs1135824 (CYP2D6*3), rs35742686 (CYP2D6*3), rs3892097, rs5030655, rs5030867, rs5030865, rs5030656, rs1065852, rs5030863, rs5030862, rs5030865, rs774671100, rs28371706 (CYP2D6*17), rs16947 (CYP2D6*17), rs61736512 (CYP2D6*29), rs1058164 (CYP2D6*29), rs16947 (CYP2D6*29), rs59421388 (CYP2D6*29), rs1135840 (CYP2D6*29), rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023.

In some embodiments, the plurality of genomic loci comprises any 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or all 82 of the genomic loci corresponding to a SNP selected from the group above.

In some embodiments, these genomic loci and their SNP status may be evaluated in conjunction with other markers known in the art as associated with therapeutic efficacy of treatment of a neuropsychiatric disorder. Exemplary types of the other markers includes but are not limited to gene expression product, epigenetic modification of genomic DNA such as methylation, and metabolic profile/signature. Archer et al., (2010) “Epigenetics and Biomarkers in the Staging of Neuropsychiatric Disorders” Neurotox Res (2010) 18: 347; Quinones et al., “Metabolomics tools for identifying biomarkers for neuropsychiatric diseases,” (2009), Neurobiology of Disease, 35, (2) 165-176.

In some embodiments, the plurality of genomic loci include SNP rs7997012, found in the HTR2A gene. The presence of SNP rs7997012 is associated with increased rate of successful response to treatment of depression with the drug citalopram. In some embodiments, the methods provided herein further include recommending, assigning, and/or administering antidepressant therapy including a selective serotonin reuptake inhibitor (SSRI) to a subject determined to carry the SNP. In one specific embodiment, the SSRI is citalopram. In some embodiments, in addition to detecting the presence of SNP rs7997012, the method further comprises detecting the presence of at least 3, 4, 5, 6, 7, 8, 9, 10, 15, 20, 30, 40, 50, 60, 70, 80, or more of the SNPs listed in Table 1 and/or Table 2.

In some embodiments, the plurality of genomic loci include SNP rs3813929, found in the HTR2C gene. The presence of SNP rs3813929 is associated with adverse response including weight gain in patients using antipsychotic medication. In some embodiments, the methods provided herein further include recommending, assigning, and/or administering antidepressant therapy including a low dose of an antipsychotic to a subject determined to carry the SNP. In one embodiment, the antipsychotic is amisulpride, clozapine, haloperidol, iloperidone, olanzapine, quetiapine, risperidone, ziprasidone, or any combination thereof. In some embodiments, in addition to detecting the presence of SNP rs3813929, the method further comprises detecting the presence of at least 3, 4, 5, 6, 7, 8, 9, 10, 15, 20, 30, 40, 50, 60, 70, 80, or more of the SNPs listed in Table 1 and/or Table 2.

In some embodiments, the plurality of genomic loci include SNP rs1045642, found in the ABCB1 gene. The presence of SNP rs1045642 is associated with adverse response in patients using antipsychotic medication. In some embodiments, the methods provided herein further include recommending, assigning, and/or administering a low dose of an antipsychotic to a subject determined to carry the SNP. In one specific embodiment, the antipsychotics is chlorpromazine. In some embodiments, in addition to detecting the presence of SNP rs1045642, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs2032583, found in the ABCB1 gene. The presence of SNP rs2032583 is associated with variable intracerebral concentrations of certain drugs and their efficacy or potential for adverse side effects, especially SSRI or tricyclic antidepressant (TCA). In some embodiments, the methods provided herein further include recommending, assigning, and/or administering antidepressant therapy comprising administration of antidepressant therapy comprising administration of a SSRI, a tricyclic antidepressant (TCA), or a combination of both, to a subject determined to carry the SNP. In one specific embodiment, the SSRI is citalopram, fluvoxamine, paroxetine, sertraline, or any combination thereof. In another specific embodiment, the TCA is amitriptyline. In some embodiments, in addition to detecting the presence of SNP rs2032583, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1800544, found in the ADRA2A gene. The presence of SNP rs1800544 is associated with efficacy of certain antidepressant medications including serotonin-norepinephrine reuptake inhibitor (SNRI). In some embodiments, the methods provided herein further include recommending, assigning, and/or administering antidepressant therapy comprising administration of a SNRI to a subject determined to carry the SNP. In one embodiment, the SNRI is milnacipran. In some embodiments, in addition to detecting the presence of SNP rs1800544, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs10994336, found in the ANK3 gene. The presence of SNP rs10994336 is associated with increased risk of developing certain neuropsychiatric disorders including bipolar disorder and schizophrenia. In some embodiments, the methods provided herein further include recommending, assigning, and/or administering antidepressant therapy comprising administration of a sodium channel modulating agent to a subject determined to carry the SNP. In one specific embodiment, the sodium channel modulating agent is lamotrigine. In some embodiments, in addition to detecting the presence of SNP rs10994336, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs6265, found in the BDNF gene. The presence of SNP rs6265 is associated with introversion, increased risk for ADHD or depression, impaired motor skills learning, and quicker mental decline in Alzheimer patients. In some embodiments, the methods provided herein further include recommending, assigning, and/or administering a low dose of an SSRI, an antipsychotic, or a combination of both, to a subject determined to carry the SNP. In one embodiment, the SSRI is paroxetine. In one embodiment, the antipsychotic is clozapine. In some embodiments, in addition to detecting the presence of SNP rs6265, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1006737, found in the CACNA1C gene. The presence of SNP rs1006737 is associated with increased risk of neuropsychiatric disorders including bipolar disorder. In some embodiments, the methods provided herein further include recommending, assigning, and/or administering a low dose of an SSRI to a subject determined to carry the SNP. In one embodiment, the SSRI is citalopram. In some embodiments, in addition to detecting the presence of SNP rs1006737, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs4680, found in the COMT gene. The presence of SNP rs4680 is associated with reduced activity of the COMT enzyme responsible for metabolizing dopamine in the brain's prefrontal cortex. In some embodiments, the methods provided herein further include recommending, assigning, and/or administering an antidepressant therapy including an SSRI, an SNRI, or a combination of both, to a subject determined to carry the SNP. In one embodiment, the SSRI is paroxetine. In one embodiment, the SNRI is venlafaxine. In some embodiments, in addition to detecting the presence of SNP rs4680, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs2470890, found in the CYP1A2 gene. The presence of SNP rs2470890 is associated with increased side effect to certain antipsychotics drugs. In some embodiments, the methods provided herein further include recommending, assigning, and/or administering a low dose of an antipsychotic to a subject determined to carry the SNP. In a specific embodiment, the antipsychotics is clozapine. In some embodiments, in addition to detecting the presence of SNP rs2470890, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs2069514, found in the CYP1A2 gene. The presence of SNP rs2069514 is associated with increased adverse response to antidepressant medication including antipsychotics. In some embodiments, the methods provided herein further include recommending, assigning, and/or administering SNP antidepressant therapy comprising administration of a low dose of antipsychotic to a subject determined to carry the SNP. In some embodiments, in addition to detecting the presence of SNP rs2069514, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs35694136, found in the CYP1A2 gene. The presence of SNP rs35694136 is associated with increased adverse response to antidepressant medication including antipsychotics. In some embodiments, the methods provided herein further include recommending, assigning, and/or administering antidepressant therapy comprising administration of a low dose of antipsychotic to a subject determined to carry the SNP. In some embodiments, in addition to detecting the presence of SNP rs35694136, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs2069526, found in the CYP1A2 gene. The presence of SNP rs2069526 is associated with decreased activity of cytochrome P450 1A2 and adverse reaction to SSRI medications. In some embodiments, the methods provided herein further include recommending, assigning, and/or administering an antidepressant therapy including a low dose of an SSRI to a subject determined to carry the SNP. In one specific embodiment, the SSRI is escitalopram. In some embodiments, in addition to detecting the presence of SNP rs2069526, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs762551, found in the CYP1A2 gene. The presence of SNP rs762551 is associated with the high inducibility form of the enzyme P450 1A2. In some embodiments, the methods provided herein further include recommending, assigning, and/or administering antidepressant therapy including a high dose of an SSRI to a subject determined to carry the SNP. In one specific embodiment, the SSRI is paroxetine. In some embodiments, in addition to detecting the presence of SNP rs762551, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs72547513, found in the CYP1A2 gene. The presence of SNP rs72547513 is associated with reduced enzymatic activity of cytochrome P450 1A2. In some embodiments, the methods provided herein further include recommending, assigning, and/or administering a low dose of a pharmaceutical agent that is metabolized by cytochrome P450 1A2 to a subject determined to carry the SNP. In some embodiments, in addition to detecting the presence of SNP rs72547513, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs2279343, found in the CYP2B6 gene. The presence of SNP rs2279343 is associated with increased risk of developing substance abuse such as heroin abuse. In some embodiments, the methods provided herein further include recommending, assigning, and/or administering therapy for substance abuse to a subject determined to carry the SNP. In one specific embodiment, the substance is heroin, and the therapy comprises administering a high dose of methadone. In one specific embodiment, the substance is nicotine, and the therapy comprises administering bupropion. In some embodiments, in addition to detecting the presence of SNP rs2279343, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs3211371, found in the CYP2B6 gene. The presence of SNP rs3211371 is associated with increased risk of developing substance abuse such as heroin abuse. In some embodiments, the methods provided herein further include recommending, assigning, and/or administering non-heroin therapy including a high dose of methadone to a subject determined to carry the SNP. In some embodiments, in addition to detecting the presence of SNP rs3211371, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs3745274, found in the CYP2B6 gene. The presence of SNP rs3745274 is associated with increased risk of developing substance abuse such as heroin abuse. In some embodiments, the methods provided herein further include recommending, assigning, and/or administering therapy for substance abuse to a subject determined to carry the SNP. In one specific embodiment, the substance is heroin, and the therapy comprises administering a high dose of methadone. In another specific embodiment, the substance is nicotine, and the therapy comprises administering bupropion. In some embodiments, in addition to detecting the presence of SNP rs3745274, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs2279343, found in the CYP2B6 gene. The presence of SNP rs2279343 is associated with increased risk of developing substance abuse such as heroin or nicotine abuse. In some embodiments, the methods provided herein further include recommending, assigning, and/or administering therapy for substance abuse to a subject determined to carry the SNP. In one specific embodiment, wherein the substance is heroin, and the therapy comprises administering a high dose of methadone. In another specific embodiment, wherein the substance is nicotine, and the therapy comprises administering bupropion. In yet another specific embodiment, the method comprises assigning antidepressant therapy comprising administration of mirtazapine. In some embodiments, in addition to detecting the presence of SNP rs2279343, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs4244285, found in the CYP2C19 gene. The presence of SNP rs4244285 is associated with reduced activity of P450 2C19. In some embodiments, the methods provided herein further include recommending, assigning, and/or administering antidepressant therapy including a low dose of a TCA, a SSRI, or a combination of both, to a subject determined to carry the SNP. In one specific embodiment, the TCA is amitriptyline. In one specific embodiment, the SSRI is citalopram, escitalopram, or a combination of both. In some embodiments, in addition to detecting the presence of SNP rs4244285, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs17878459, found in the CYP2C19 gene. The presence of SNP rs17878459 is associated with inactivity of cytochrome P450 2C19. In some embodiments, the methods provided herein further include recommending, assigning, and/or administering antidepressant therapy including a low dose of an antipsychotic to a subject determined to carry the SNP. In some embodiments, in addition to detecting the presence of SNP rs17878459, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs4986893, found in the CYP2C19 gene. The presence of SNP rs4986893 is associated with reduced activity of P450 2C19. In some embodiments, the methods provided herein further include recommending, assigning, and/or administering antidepressant therapy including a low dose of an SSRI to a subject determined to carry the SNP. In one specific embodiment, the SSRI is citalopram, escitalopram, or a combination of both. In some embodiments, in addition to detecting the presence of SNP rs4986893, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs57081121, found in the CYP2C19 gene. The presence of SNP rs57081121 is associated with reduced activity of P450 2C19. In some embodiments, the methods provided herein further include recommending, assigning, and/or administering antidepressant therapy including a low dose of a SSRI to a subject determined to carry the SNP. In one specific embodiment, the SSRI is citalopram, escitalopram, or a combination of both. In some embodiments, in addition to detecting the presence of SNP rs57081121, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs28399504, found in the CYP2C19 gene. The presence of SNP rs28399504 is associated with reduced activity of P450 2C19. In some embodiments, the methods provided herein further include recommending, assigning, and/or administering antidepressant therapy including a low dose of a SSRI to a subject determined to carry the SNP. In one specific embodiment, the SSRI is citalopram, escitalopram, or a combination of both. In some embodiments, in addition to detecting the presence of SNP rs28399504, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs56337013, found in the CYP2C19 gene. The presence of SNP rs56337013 is associated with reduced activity of P450 2C19 In some embodiments, the methods provided herein further include recommending, assigning, and/or administering a low dose of a pharmaceutical agent that is metabolized by cytochrome P450 2C19 (CYP2C19) to a subject determined to carry the SNP. In some embodiments, in addition to detecting the presence of SNP rs56337013, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs72552267, found in the CYP2C19 gene. The presence of SNP rs72552267 is associated with reduced activity of P450 2C19. In some embodiments, the methods provided herein further include recommending, assigning, and/or administering a low dose of a pharmaceutical agent that is metabolized by cytochrome P450 2C19 (CYP2C19) to a subject determined to carry the SNP. In some embodiments, in addition to detecting the presence of SNP rs72552267, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs72558186, found in the CYP2C19 gene. The presence of SNP rs72558186 is associated with reduced activity of P450 2C19. In some embodiments, the methods provided herein further include recommending, assigning, and/or administering a low dose of a pharmaceutical agent that is metabolized by cytochrome P450 2C19 (CYP2C19) to a subject determined to carry the SNP. In some embodiments, in addition to detecting the presence of SNP rs72558186, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs41291556, found in the CYP2C19 gene. The presence of SNP rs41291556 is associated with reduced activity of P450 2C19. In some embodiments, the methods provided herein further include recommending, assigning, and/or administering a low dose of a pharmaceutical agent that is metabolized by cytochrome P450 2C19 (CYP2C19) to a subject determined to carry the SNP. In some embodiments, in addition to detecting the presence of SNP rs41291556, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs17884712, found in the CYP2C19 gene. The presence of SNP rs17884712 is associated with reduced activity of P450 2C19. In some embodiments, the methods provided herein further include recommending, assigning, and/or administering a low dose of a pharmaceutical agent that is metabolized by cytochrome P450 2C19 (CYP2C19) to a subject determined to carry the SNP. In some embodiments, in addition to detecting the presence of SNP rs17884712, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs6413438, found in the CYP2C19 gene. The presence of SNP rs6413438 is associated with reduced activity of P450 2C19. In some embodiments, the methods provided herein further include recommending, assigning, and/or administering a low dose of a pharmaceutical agent that is metabolized by cytochrome P450 2C19 (CYP2C19) to a subject determined to carry the SNP. In some embodiments, in addition to detecting the presence of SNP rs6413438, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs12248560, found in the CYP2C19 gene. The presence of SNP rs12248560 is associated with extensive metabolism (“ultra-metabolizer”) of drugs normally metabolized by P450 2C19. In some embodiments, the methods provided herein further include recommending, assigning, and/or administering antidepressant therapy including a low dose of a SSRI or a TCA to a subject determined to carry the SNP. In one specific embodiment, the SSRI is citalopram, escitalopram, or a combination of both. In one specific embodiment, the TCA is amitriptyline, clomipramine, or a combination of both. In some embodiments, in addition to detecting the presence of SNP rs12248560, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs12769205, found in the CYP2C19 gene. The presence of SNP rs12769205 is associated with reduced activity of P450 2C19. In some embodiments, the methods provided herein further include recommending, assigning, and/or administering antidepressant therapy including a low dose of a SSRI or a TCA to a subject determined to carry the SNP. In one specific embodiment, the SSRI is sertraline, escitalopram, or a combination of both. In one specific embodiment, the TCA is amitriptyline or imipramine. In some embodiments, in addition to detecting the presence of SNP rs12769205, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs3758581, found in the CYP2C19 gene. The presence of SNP rs3758581 is associated with reduced activity of P450 2C19 In some embodiments, the methods provided herein further include recommending, assigning, and/or administering antidepressant therapy including a low dose of an SSRI, a TCA, or a combination of both, to a subject determined to carry the SNP. In one specific embodiment, the SSRI is sertraline, escitalopram, or a combination of both. In one specific embodiment, the TCA is amitriptyline, imipramine, or a combination of both. In some embodiments, in addition to detecting the presence of SNP rs3758581, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1799853, found in the CYP2C9 gene. The presence of SNP rs1799853 is associated with adverse drug response resulting from poor metabolism of certain drugs including NSAID drugs such as warfarin and valproic acid. In some embodiments, the methods provided herein further include recommending, assigning, and/or administering a psychotropic therapy including a low dose of valproic acid to a subject determined to carry the SNP. In some embodiments, in addition to detecting the presence of SNP rs1799853, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1057910, found in the CYP2C9 gene. The presence of SNP rs1057910 is associated with adverse drug response resulting from poor metabolism of certain drugs including NSAID drugs such as warfarin and valproic acid. In some embodiments, the methods provided herein further include recommending, assigning, and/or administering a psychotropic therapy including a low dose of a TCA, valproic acid, or a combination of both, to a subject determined to carry the SNP. In one specific embodiment, the TCA is trimipramine, doxepin, or a combination of both. In some embodiments, in addition to detecting the presence of SNP rs1057910, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs56165452, found in the CYP2C9 gene. The presence of SNP rs56165452 is associated with reduced activity of P450 2C9. In some embodiments, the methods provided herein further include recommending, assigning, and/or administering a low dose of a pharmaceutical agent that is metabolized by cytochrome P450 2C9 to a subject determined to carry the SNP. In some embodiments, in addition to detecting the presence of SNP rs56165452, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs28371686, found in the CYP2C9 gene. The presence of SNP rs28371686 is associated with reduced activity of P450 2C9. In some embodiments, the methods provided herein further include recommending, assigning, and/or administering a low dose of a pharmaceutical agent that is metabolized by cytochrome P450 2C9 to a subject determined to carry the SNP. In some embodiments, in addition to detecting the presence of SNP rs28371686, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs9332131, found in the CYP2C9 gene. The presence of SNP rs9332131 is associated with inactivity of P450 2C9. In some embodiments, the methods provided herein further include recommending, assigning, and/or administering a low dose of an antiepileptic drug (AED) to a subject determined to carry the SNP. In one specific embodiment, the AED is phenytoin. In some embodiments, in addition to detecting the presence of SNP rs9332131, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs7900194, found in the CYP2C9 gene. The presence of SNP rs7900194 is associated with reduced activity of P450 2C9. In some embodiments, the methods provided herein further include recommending, assigning, and/or administering a low dose of an AED to a subject determined to carry the SNP. In one specific embodiment, the AED is phenytoin. In some embodiments, in addition to detecting the presence of SNP rs7900194, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs28371685, found in the CYP2C9 gene. The presence of SNP rs28371685 is associated with reduced activity of P450 2C9. In some embodiments, the methods provided herein further include recommending, assigning, and/or administering a low dose of a pharmaceutical agent that is metabolized by cytochrome P450 2C9 (CYP2C9) to a subject determined to carry the SNP. In some embodiments, in addition to detecting the presence of SNP rs28371685, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs72558187, found in the CYP2C9 gene. The presence of SNP rs72558187 is associated with reduced activity of P450 2C9. In some embodiments, the methods provided herein further include recommending, assigning, and/or administering a low dose of AED to a subject determined to carry the SNP. In one specific embodiment, the AED is phenytoin. In some embodiments, in addition to detecting the presence of SNP rs72558187, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1135840, found in the CYP2D6 gene. In some embodiments, the methods provided herein further include recommending, assigning, and/or administering antidepressant therapy including a TCA, an SSRI, a norepinephrine reuptake inhibitor (NRI), or any combination thereof, to a subject determined to carry the SNP. In one specific embodiment, the TCA is imipramine, amitriptyline, trimipramine, clomipramine, desipramine, doxepin, or any combination thereof. In one specific embodiment, the SSRI is paroxetine, citalopram, escitalopram, or any combination thereof. In one specific embodiment, the NRI is atomoxetine. In some embodiments, in addition to detecting the presence of SNP rs1135840, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs16947, found in the CYP2D6 gene. The presence of SNP rs16947 is associated with extensive metabolism of drugs normally metabolized by P450 2D6. In some embodiments, the methods provided herein further include recommending, assigning, and/or administering antidepressant therapy including a TCA, an SSRI, an NRI, or any combination thereof, to a subject determined to carry the SNP. In one specific embodiment, the TCA is imipramine, amitriptyline, trimipramine, clomipramine, desipramine, doxepin, or any combination thereof. In one specific embodiment, the SSRI is paroxetine, citalopram, escitalopram, or any combination thereof. In one specific embodiment, the NRI is atomoxetine. In some embodiments, in addition to detecting the presence of SNP rs16947, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1135824, found in the CYP2D6 gene. The presence of SNP rs1135824 is associated with inactivity of P450 2D6. In some embodiments, the methods provided herein further include recommending, assigning, and/or administering antidepressant therapy including a low dose of an SSRI, a TCA, an NRI, or any combination thereof, to a subject determined to carry the SNP. In one specific embodiment, the SSRI is paroxetine, citalopram, fluvoxamine, fluoxetine, escitalopram, or any combination thereof. In one specific embodiment, the TCA is imipramine, amitriptyline, trimipramine, clomipramine, desipramine, doxepin, trimipramine, nortriptyline, or any combination thereof. In one specific embodiment, the NRI is atomoxetine. In some embodiments, in addition to detecting the presence of SNP rs1135824, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs35742686, found in the CYP2D6 gene. The presence of SNP rs35742686 is associated with inactivity of P450 2D6. In some embodiments, the methods provided herein further include recommending, assigning, and/or administering antidepressant therapy including a low dose of an SSRI, a TCA, an NRI, or any combination thereof, to a subject determined to carry the SNP. In one specific embodiment, the TCA is imipramine, amitriptyline, trimipramine, clomipramine, desipramine, doxepin, trimipramine, nortriptyline, or any combination thereof. In one specific embodiment, the SSRI is paroxetine, citalopram, fluvoxamine, fluoxetine, escitalopram, or any combination thereof. In one specific embodiment, the NRI is atomoxetine. In some embodiments, in addition to detecting the presence of SNP rs35742686, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs3892097, found in the CYP2D6 gene. The presence of SNP rs3892097 is associated with inactivity of P450 2D6. In some embodiments, the methods provided herein further include recommending, assigning, and/or administering antidepressant therapy including a low dose of an SSRI, a TCA, an NRI, or any combination thereof, to a subject determined to carry the SNP. In one specific embodiment, the SSRI is paroxetine, citalopram, fluvoxamine, fluoxetine, escitalopram, or any combination thereof. In one specific embodiment, the TCA is imipramine, amitriptyline, trimipramine, clomipramine, desipramine, doxepin, trimipramine, nortriptyline, or any combination thereof. In one specific embodiment, the NRI is atomoxetine. In some embodiments, in addition to detecting the presence of SNP rs3892097, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs5030655, found in the. The presence of SNP rs5030655 is associated with reduced activity of P450 2D6. In some embodiments, the methods provided herein further include recommending, assigning, and/or administering antidepressant therapy including a low dose of an SSRI, a TCA, an NRI, or any combination thereof, to a subject determined to carry the SNP. In one specific embodiment, the SSRI is paroxetine, citalopram, fluvoxamine, fluoxetine, escitalopram, or any combination thereof. In one specific embodiment, the TCA is imipramine, amitriptyline, trimipramine, clomipramine, desipramine, doxepin, trimipramine, nortriptyline, or any combination thereof. In one specific embodiment, the NRI is atomoxetine. In some embodiments, in addition to detecting the presence of SNP rs5030655, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs5030867, found in the CYP2D6 gene. The presence of SNP rs5030867 is associated with inactivity of P450 2D6. In some embodiments, the methods provided herein further include recommending, assigning, and/or administering a low dose of an antipsychotic to a subject determined to carry the SNP. In some embodiments, in addition to detecting the presence of SNP rs5030867, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs5030865, found in the CYP2D6 gene. The presence of SNP rs5030865 is associated with inactivity of P450 2D6. In some embodiments, the methods provided herein further include recommending, assigning, and/or administering antidepressant therapy including a low dose of an antipsychotic to a subject determined to carry the SNP. In one specific embodiment, the antipsychotics is risperidone. In some embodiments, in addition to detecting the presence of SNP rs5030865, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs5030656, found in the CYP2D6 gene. The presence of SNP rs5030656 is associated with poor metabolism of pharmaceutical agents normally metabolized by P450 2D6. In some embodiments, the methods provided herein further include recommending, assigning, and/or administering a low dose of a pharmaceutical agent that is metabolized by cytochrome P450 2D6 to a subject determined to carry the SNP. In some embodiments, in addition to detecting the presence of SNP rs5030656, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1065852, found in the CYP2D6 gene. The presence of SNP rs1065852 is associated with poor metabolism of pharmaceutical agents normally metabolized by P450 2D6. In some embodiments, the methods provided herein further include recommending, assigning, and/or administering antidepressant therapy including a low dose of an SSRI, a TCA, an NRI, an antipsychotic, or any combination thereof, to a subject determined to carry the SNP. In one specific embodiment, the SSRI is paroxetine, citalopram, fluvoxamine, fluoxetine, escitalopram, or any combination thereof. In one specific embodiment, the TCA is imipramine, amitriptyline, trimipramine, clomipramine, desipramine, doxepin, nortriptyline, or any combination thereof. In one specific embodiment, the NRI is atomoxetine. In some embodiments, in addition to detecting the presence of SNP rs1065852, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs5030863, found in the CYP2D6 gene. The presence of SNP rs5030863 is associated with inactivity of P450 2D6. In some embodiments, the methods provided herein further include recommending, assigning, and/or administering a low dose of a pharmaceutical agent that is metabolized by cytochrome P450 2D6 (CYP2D6) to a subject determined to carry the SNP. In some embodiments, in addition to detecting the presence of SNP rs5030863, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs5030862, found in the CYP2D6 gene. The presence of SNP rs5030862 is associated with inactivity of P450 2D6. In some embodiments, the methods provided herein further include recommending, assigning, and/or administering a low dose of a pharmaceutical agent that is metabolized by cytochrome P450 2D6 (CYP2D6) to a subject determined to carry the SNP. In some embodiments, in addition to detecting the presence of SNP rs5030862, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs5030865, found in the CYP2D6 gene. The presence of SNP rs5030865 is associated with inactivity of P450 2D6. In some embodiments, the methods provided herein further include recommending, assigning, and/or administering antidepressant therapy including a low dose of an antipsychotic to a subject determined to carry the SNP. In one specific embodiment, antipsychotics is risperidone. In some embodiments, in addition to detecting the presence of SNP rs5030865, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs774671100, found in the CYP2D6 gene. The presence of SNP rs774671100 is associated with inactivity of cytochrome P450 2D6. In some embodiments, the methods provided herein further include recommending, assigning, and/or administering a low dose of a pharmaceutical agent that is metabolized by cytochrome P450 2D6 to a subject determined to carry the SNP. In some embodiments, in addition to detecting the presence of SNP rs774671100, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs28371706, found in the CYP2D6 gene. The presence of SNP rs28371706 is associated with reduced activity of P450 2D6. In some embodiments, the methods provided herein further include recommending, assigning, and/or administering antidepressant therapy including a TCA to a subject determined to carry the SNP. In one specific embodiment, the TCA is haloperidol. In one specific embodiment, the TCA is desipramine, nortriptyline, or a combination of both, and a low dose of the TCA is administered. In some embodiments, in addition to detecting the presence of SNP rs28371706, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs61736512, found in the CYP2D6 gene. The presence of SNP rs61736512 is associated with decreased activity of P450 2D6 activity. In some embodiments, the methods provided herein further include recommending, assigning, and/or administering a low dose of a pharmaceutical agent that is metabolized by cytochrome P450 2D6 (CYP2D6) to a subject determined to carry the SNP. In some embodiments, in addition to detecting the presence of SNP rs61736512, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1058164, found in the CYP2D6 gene. The presence of SNP rs1058164 is associated with decreased activity of P450 2D6 activity. In some embodiments, the methods provided herein further include recommending, assigning, and/or administering a low dose of a pharmaceutical agent that is metabolized by cytochrome P450 2D6 (CYP2D6) to a subject determined to carry the SNP. In some embodiments, in addition to detecting the presence of SNP rs1058164, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs59421388, found in the CYP2D6 gene. The presence of SNP rs59421388 is associated with decreased activity of P450 2D6 activity. In some embodiments, the methods provided herein further include recommending, assigning, and/or administering a low dose of a pharmaceutical agent that is metabolized by cytochrome P450 2D6 (CYP2D6) to a subject determined to carry the SNP. In some embodiments, in addition to detecting the presence of SNP rs59421388, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs28371725, found in the CYP2D6 gene. The presence of SNP rs28371725 is associated with decreased activity of P450 2D6. In some embodiments, the methods provided herein further include recommending, assigning, and/or administering antidepressant therapy including a TCA, an SSRI, an SNRI, or any combination thereof, to a subject determined to carry the SNP. In one specific embodiment, the SSRI is citalopram or escitalopram. In one specific embodiment, the TCA is desipramine, aripiprazole, haloperidol, levomepromazine, quetiapine, risperidone, or any combination thereof, and a low dose of the TCA is administered. In one specific embodiment, the SSRI is desipramine, aripiprazole, haloperidol, levomepromazine, quetiapine, or any combination thereof, and a low dose of the SSRI is administered. In some embodiments, in addition to detecting the presence of SNP rs28371725, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs35599367, found in the CYP3A4 gene. The presence of SNP rs35599367 is associated with reduced expression of the CYP3A4 gene. In some embodiments, the methods provided herein further include recommending, assigning, and/or administering antidepressant therapy including a low dose of an antipsychotic to a subject determined to carry the SNP. In one specific embodiment, the antipsychotics is risperidone. In some embodiments, in addition to detecting the presence of SNP rs35599367, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs776746, found in the CYP3A5 gene. The presence of SNP rs776746 is associated with reduced activity of cytochrome P450 3A5. In some embodiments, the methods provided herein further include recommending, assigning, and/or administering a low dose of a pharmaceutical agent that is metabolized by cytochrome P450 3A5 to a subject determined to carry the SNP. In some embodiments, in addition to detecting the presence of SNP rs776746, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs10264272, found in the rs10264272 gene. The presence of SNP rs10264272 is associated with nonfunctional cytochrome P450 3A5. In some embodiments, the methods provided herein further include recommending, assigning, and/or administering a low dose of a pharmaceutical agent that is metabolized by cytochrome P450 3A5 to a subject determined to carry the SNP. In some embodiments, in addition to detecting the presence of SNP rs10264272, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs41303343, found in the CYP3A5 gene. The presence of SNP rs41303343 is associated with nonfunctional cytochrome P450 3A5. In some embodiments, the methods provided herein further include recommending, assigning, and/or administering a low dose of a pharmaceutical agent that is metabolized by cytochrome P450 3A5 to a subject determined to carry the SNP. In some embodiments, in addition to detecting the presence of SNP rs41303343, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1799732, found in the DRD2 gene. The presence of SNP rs1799732 is associated with abnormal binding of dopamine and certain antipsychotics. It is also associated with adverse reaction and reduced efficacy of certain antipsychotics. In some embodiments, the methods provided herein further include recommending, assigning, and/or administering an antipsychotic to a subject determined to carry the SNP. In one specific embodiment, the antipsychotic is aripiprazole, bromperidol, chlorpromazine, clozapine, nemonapride, olanzapine, risperidone, or any combination thereof. In one specific embodiment, the antipsychotic is risperidone, and a low dose of the antipsychotic is administered. In yet another embodiment, the method comprises assigning therapy for tobacco use disorder comprising administration of a non-nicotine replacement. In some embodiments, in addition to detecting the presence of SNP rs1799732, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs2832407, found in the GRIK1 gene. The presence of SNP rs2832407 is associated with increased risk of alcohol abuse. In some embodiments, the methods provided herein further include recommending, assigning, and/or administering therapy for alcohol abuse including a low dose of topiramate to a subject determined to carry the SNP. In some embodiments, in addition to detecting the presence of SNP rs2832407, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1061235, found in the HLA-A gene. The presence of SNP rs1061235 is associated with carbamazepine-induced adverse hypersensitivity reactions. In some embodiments, the methods provided herein further include recommending, assigning, and/or administering a low dose of an anticonvulsant or an AED to a subject determined to carry the SNP. In one specific embodiment, the anticonvulsant is carbamazepine, oxcarbazepine, lamotrigine, or any combination thereof. In one specific embodiment, the AED is phenytoin. In some embodiments, in addition to detecting the presence of SNP rs1061235, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs2395148, found in the HLA-B gene. The presence of SNP rs2395148 is associated with carbamazepine-induced adverse hypersensitivity reactions. In some embodiments, the methods provided herein further include recommending, assigning, and/or administering a lose dose of an anticonvulsant or an AED to a subject determined to carry the SNP. In one specific embodiment, the anticonvulsant is carbamazepine, oxcarbazepine, lamotrigine, or any combination thereof. In one specific embodiment, the AED is phenytoin. In some embodiments, in addition to detecting the presence of SNP rs2395148, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs489693, found in the MC4R gene. The presence of SNP rs489693 is associated with adverse response to certain antipsychotics. In some embodiments, the methods provided herein further include recommending, assigning, and/or administering a low dose of an antipsychotic to a subject determined to carry the SNP. In one specific embodiment, the antipsychotic is amisulpride, aripiprazole, clozapine, haloperidol, olanzapine, paliperidone, quetiapine, risperidone, ziprasidone, or any combination thereof. In some embodiments, in addition to detecting the presence of SNP rs489693, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1801131, found in the MTHFR gene. The presence of SNP rs1801131 is associated with increased risk of depression, bipolar disorder, negative symptoms of schizophrenia, substance abuse, and autism. In some embodiments, the methods provided herein further include recommending, assigning, and/or administering antidepressant therapy including a low dose of an antipsychotic to a subject determined to carry the SNP. In one specific embodiment, the antipsychotic is olanzapine, clozapine, or a combination of both. In some embodiments, the methods provided herein further include recommending, assigning, and/or administering anti-depression therapy including 1-methylfolate, vitamin B-complex, or a combination of both, to a subject determined to carry the SNP. In yet another specific embodiment, the method further comprises assigning therapy for cocaine abuse comprising administration of disulfiram. In some embodiments, in addition to detecting the presence of SNP rs1801131, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1801133, found in the MTHFR gene. The presence of SNP rs1801133 is associated with increased risk of depression, bipolar disorder, negative symptoms of schizophrenia, substance abuse, and autism. In some embodiments, the methods provided herein further include recommending, assigning, and/or administering antidepressant therapy including a low dose of an antipsychotic to a subject determined to carry the SNP. In one specific embodiment, the antipsychotic is olanzapine, clozapine, or a combination of both. In some embodiments, the methods provided herein further include recommending, assigning, and/or administering anti-depression therapy including 1-methylfolate, vitamin B-complex, or a combination of both, to a subject determined to carry the SNP. In some embodiments, the methods provided herein further include recommending, assigning, and/or administering therapy for cocaine abuse including disulfiram to a subject determined to carry the SNP. In some embodiments, in addition to detecting the presence of SNP rs1801133, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1799971, found in the OPRM1 gene. The presence of SNP rs1799971 is associated with increased risk of substance abuse including alcohol and nicotine. In some embodiments, the methods provided herein further include recommending, assigning, and/or administering therapy for substance abuse to a subject determined to carry the SNP. In one specific embodiment, the substance is tobacco, and the therapy comprises administering a nicotine-replacement. In one specific embodiment, the substance is opioid, and the therapy comprises administering a low dose of methadone. In some embodiments, in addition to detecting the presence of SNP rs1799971, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs25531, found in the SLC6A4 gene. The presence of SNP rs25531 is associated with reduced reuptake of serotonin, less satisfactory response to SSRI-based treatment, lower rate of stress resilience, and higher rate of PTSD. In some embodiments, the methods provided herein further include recommending, assigning, and/or administering antidepressant therapy including a low dose of SSRI to a subject determined to carry the SNP. In one specific embodiment, the SSRI is fluoxetine, citalopram, or a combination of both. In some embodiments, in addition to detecting the presence of SNP rs25531, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs63749047, found in the SLC6A4 gene. The presence of SNP rs63749047 is associated with reduced reuptake of serotonin, less satisfactory response to SSRI-based treatment, lower rate of stress resilience, and higher rate of PTSD. In some embodiments, the methods provided herein further include recommending, assigning, and/or administering antidepressant therapy including a low dose of SSRI to a subject determined to carry the SNP. In one specific embodiment, the SSRI is citalopram, escitalopram, fluoxetine, fluvoxamine, paroxetine, sertraline, or any combination thereof. In some embodiments, in addition to detecting the presence of SNP rs63749047, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs2011425, found in the UGT1A4 gene. The presence of SNP rs2011425 is associated with adverse responses to certain anticonvulsant drugs. In some embodiments, the methods provided herein further include recommending, assigning, and/or administering a low dose of lamotrigine, asenapine, trifluoperazine, or any combination thereof, to a subject determined to carry the SNP. In some embodiments, in addition to detecting the presence of SNP rs2011425, the method further comprises detecting the presence of at least 3, 4, 5, 6, 7, 8, 9, 10, 15, 20, 30, 40, 50, 60, 70, 80, or more SNPs listed in Table 1 and/or Table 2.

In some embodiments, the plurality of genomic loci include SNP rs1902023, found in the UGT2B15 gene. The presence of SNP rs1902023 is associated with adverse response to certain benzodiazepine (BZD) drugs. In some embodiments, the methods provided herein further include recommending, assigning, and/or administering psychotropic therapy including a low dose of a BZD to a subject determined to carry the SNP. In one specific embodiment, the BZD is clonazepam, diazepam, lorazepam, oxazepam, temazepam, or any combination thereof. In some embodiments, in addition to detecting the presence of SNP rs1902023, the method further comprises detecting the presence of at least 3, 4, 5, 6, 7, 8, 9, 10, 15, 20, 30, 40, 50, 60, 70, 80, or more SNPs listed in Table 1 and/or Table 2.

TABLE 1
Example SNPs with known pharmacogenetic associations
for the treatment of neuropsychiatric disorders.

| SNP | Position | Gene |
|---|---|---|
| rs7997012 | chr13: 46837850 | HTR2A |
| rs3813929 | chr10: 114584047 | HTR2C |
| rs1045642 | chr7: 87509329 | ABCB1 |
| rs2032583 | chr7: 87531245 | ABCBI |
| rs1800544 | chr10: 111076745 | ADRA2A |
| rs10994336 | chr10: 60420054 | ANK3 |
| rs6265 | chr11: 27658369 | BDNF |
| rs1006737 | chr12: 2236129 | CACNA1C |
| rs4680 | chr22: 19963748 | COMT |
| rs1799732 | chr11: 113475530 | DRD2 |
| rs2832407 | chr21: 29595188 | GRIK1 |
| rs489693 | chr18: 60215554 | MC4R |
| rs1801131 | chr1: 11794419 | MTHFR |
| rs1801133 | chr1: 11796321 | MTHFR |
| rs1799971 | chr6: 154039662 | OPRM1 |
| rs25531 | chr17: 30237328 | SLC6A4 |
| rs63749047 | chr17: 28564340 | SLC6A4 |

TABLE 2
Additional example SNPs with known pharmacogenetic
associations for the treatment of neuropsychiatric disorders.

| SNP | Position | Gene |
|---|---|---|
| rs2470890 | chr15: 74755085 | CYP1A2 |
| rs2069514 | chr15: 74745879 | CYP1A2 |
| rs35694136 | chr15: 74747272 | CYP1A2 |
| rs2069526 | chr15: 74749000 | CYP1A2 |
| rs762551 | chr15: 74749576 | CYP1A2 |
| rs72547513 | chr15: 74750296 | CYP1A2 |
| rs2279343 | chr19: 41009358 | CYP2B6 |
| rs3211371 | chr19: 41016810 | CYP2B6 |
| rs3745274 | chr19: 41006936 | CYP2B6 |
| rs2279343 | chr19: 41009358 | CYP2B6 |
| rs4244285 | chr10: 94781859 | CYP2C19 |
| rs17878459 | chr10: 94775165 | CYP2C19 |
| rs4986893 | chr10: 94780653 | CYP2C19 |
| rs57081121 | chr10: 94780653 | CYP2C19 |
| rs28399504 | chr10: 94762706 | CYP2C19 |
| rs56337013 | chr10: 94852738 | CYP2C19 |
| rs72552267 | chr10: 94775453 | CYP2C19 |
| rs72558186 | chr10: 94781999 | CYP2C19 |
| rs41291556 | chr10: 94775416 | CYP2C19 |
| rs17884712 | chr10: 94775489 | CYP2C19 |
| rs6413438 | chr10: 94781858 | CYP2C19 |
| rs12248560 | chr10: 94761900 | CYP2C19 |
| rs12769205 | chr10: 94775367 | CYP2C19 |
| rs3758581 | chr10: 94842866 | CYP2C19 |
| rs1799853 | chr10: 94942290 | CYP2C9 |
| rs1057910 | chr10: 94981296 | CYP2C9 |
| rs56165452 | chr10: 94981297 | CYP2C9 |
| rs28371686 | chr10: 94981301 | CYP2C9 |
| rs9332131 | chr10: 94949282 | CYP2C9 |
| rs7900194 | chr10: 94942309 | CYP2C9 |
| rs28371685 | chr10: 94981224 | CYP2C9 |
| rs72558187 | chr10: 94941958 | CYP2C9 |
| rs1135840 | chr22: 42126611 | CYP2D6 |
| rs16947 | chr22: 42127941 | CYP2D6 |
| rs1135824 | chr22: 42129042 | CYP2D6 |
| rs35742686 | chr22: 42128242 | CYP2D6 |
| rs3892097 | chr22: 421289454 | CYP2D6 |
| rs5030655 | chr22: 42129084 | CYP2D6 |
| rs5030867 | chr22: 42127856 | CYP2D6 |
| rs5030865 | chr22: 42129033 | CYP2D6 |
| rs5030656 | chr22: 42128174 | CYP2D6 |
| rs1065852 | chr22: 42130692 | CYP2D6 |
| rs5030863 | chr22: 42525912 | CYP2D6 |
| rs5030862 | chr22: 42130668 | CYP2D6 |
| rs5030865(T) | chr22: 42129033 | CYP2D6 |
| rs774671100 | chr22: 42130655 | CYP2D6 |
| rs28371706 | chr22: 42129770 | CYP2D6 |
| rs61736512 | chr22: 42129132 | CYP2D6 |
| rs1058164 | chr22: 42129130 | CYP2D6 |
| rs59421388 | chr22: 42127608 | CYP2D6 |
| rs28371725 | chr22: 42127803 | CYP2D6 |
| rs35599367 | chr7: 99768693 | CYP3A4 |
| rs776746 | chr7: 99672916 | CYP3A5 |
| rs10264272 | chr7: 99665212 | CYP3A5 |
| rs41303343 | chr7: 99652771 | CYP3A5 |
| rs1061235 | chr6: 29945521 | HLA-A |
| rs2395148 | chr6: 32353777 | HLA-B |
| rs2011425 | chr2: 233718962 | UGT1A4 |
| rs1902023 | chr4: 68670366 | UGT2B15 |

### VII. Patient-Specific Report

In treating neuropsychiatric disorders, it is critical for clinicians to have a patient-specific report that can provide personalized treatment recommendations based on interpretive analysis of a patient's genotype. Although various research and clinical studies have looked for diagnostic and therapeutic indicators in an almost overwhelming variety of genomic markers, gene expression markers and protein markers, this vast and growing body of data has proven difficult to interpret. Most physicians are unable to synthesize the tremendous amount of information on possible risk factors and indicators in order to apply this information clinically to diagnose and/or treat patients. As such, there is a need for patient-specific reports to enable a medical professional to apply the most relevant genetic, epigenetic, transcriptomic, proteomic and functional imaging tests in a meaningful manner to their patients.

U.S. Pat. No. 8,355,927 illustrates an exemplary method and report for presenting genetic information that is patient-specific and relevant to treatment of neuropsychiatric disorders, as well as treatment resistant psychiatric disorders, to aid in patient treatment in a phenotype, genotype or biomarker-specific manner. The patent is herein incorporated by reference in its entirety. The methods and reports examine biomarkers for dysfunction of three brain function areas (axes) relevant to treating neuropsychiatric disorders and provide interpretive comments to aid in treatment. Combining biomarker information from each of the three axes (the autonomic arousal axis, the emotional valence, attention, reward and executive brain function axis, and the long-term potentiation and long-term depression (LTP-LTD) function axis), such as SNP composition of relevant genes, provides an comprehensive and effective means for directing treatment of neuropsychiatric disorders including treatment resistant disorders (TRD).

In particular, the method as disclosed in U.S. Pat. No. 8,355,927 uses a computer processor to generate a patient-specific report to treat a neuropsychiatric disorder including depressive disorders, bipolar disorder, anxiety disorders, PTSD, schizophrenia, autism, ADHD, and treatment resistant forms of these disorders. In general, the method comprises (1) collecting, using the processor, the results of a biomarker test specific to a patient for at least one biomarker (e.g., a pharmacokinetic SNP variant of a gene that is involved in the metabolism pathway of a drug) for dysfunction in each of the three above neuropsychiatric axes; (2) selecting, using the processor, an interpretive comment based on the patient's results of the biomarker test; (3) organizing, using the processor, the results of the biomarker tests and one or more interpretive comments in a patient-specific neuropsychiatric report; and (4) presenting the patient-specific neuropsychiatric report, wherein the report comprises an interpretive analysis of the neuropsychological significance of the result of any biomarker test for dysfunction collected, wherein the interpretive analysis comprises interpretive comments that include a description of an association with a disorder or a dysfunction of brain activity or a disorder and dysfunction of brain activity based on the biomarker test results.

In a specific example, in treating a TRD of a patient with a neuropsychiatric disorder, A set of 6 core genomic loci were first selected from 6 genes associated with the condition, and SNP genotyping assays were conducted to screen these core genomic loci, corresponding to SLC6A4 (variants of a single-nucleotide polymorphism of Serotonin Transporter); MTHFR (variants in methylenetetrahydrofolate reductase); COMT (variants in Catechol-O-Methyl Transferase); DRD2 (variants of Dopamine receptor D2); CACNA1C (variants of L-type voltage-gated calcium channel); ANK3 (variants of ankyrin 3), HTR2C (5-hydroxytryptamine (serotonin) receptor 2C), CYP2D6, CYP2C 19, CYP3A4 (variants of cytochrome P450). In this example, members of this core group are relevant in part because they indicate a possible therapeutic decision. Genes present on the patient report were those that are relevant to patient treatment outcome, including the avoidance of side effects, increasing effectiveness of drug therapies, or the like. Drugs such as psychotropic agents are of particular interest, and the accompanying interpretive comments (if included) may be related to the influence of one or more of the core epistatic group on such drugs.

The report illustrated herein included not only the results of the genotyping assays, but also interpretive comments suggesting a treatment based on identified SNPs, such as those relevant to therapeutic efficacy and drug response. The various types of interpretive comments that may be included in the report include: physiological and/or clinical significance, association studies, current research findings, pharmacological implications, and the like. The information provided by the interpretive comments may be based on medical and scientific research, including both published and unpublished data. Here, interpretive comments included in the report for the serotonin neurotransmission loci (e.g., the SNP functional variant of a single-nucleotide polymorphism (rs25531) in 5-HTTLPR (serotonin-transporter-linked promoter region of the serotonin transporter gene)) included descriptions of the gene or region of the gene examined by the genotyping assay (“the gene SLC6A4 encodes the 5-HTT, a membrane protein that transports serotonin from synaptic spaces into presynaptic neurons”), as well as information specifically relevant to the drug response/treatment response (“pharmacodynamic studies of the serotonin transporter gene suggest that patients with the S/S genotype do not respond as well to SSRI antidepressants and may experience more side effects,” “in SSRI non responders who exhibit the S/S allele, consideration should be given to use of a non-SSRI,” etc.). References may also be provided.

In addition, in this example, the report also included an indexing or weighting system providing a confidence level for the provided interpretive comments. In general, all or a subset of the interpretive comments may be indexed with an indicator (which may also be referred to as an “index”) providing a confidence level for the interpretive comment. For example, in some cases the interpretive comments may include a description or mention of the results of one or more association studies relevant to the patient's biomarker test results. An index may provide weighting context by indicating the appropriateness of the association study to support the interpretive comment. Thus, the report may indicate after the study mentioned a “grade” applied to the study (or to other interpretive comments) indicating the nature of the study (e.g., multiple studies reporting or supporting the provided association with the biomarker, a meta-analysis of multiple or single genome-wide studies supporting the association, multiple studies supporting the association, and a single study supporting the association). A letter, number, symbol, color, or other grade may be used. In some variations the indexing may rank the confidence level (e.g., having grades A through D, 1-4, etc.), with the strongest support being ranked “highest.” A key to the indexing or weighting may be provided as part of the report.

The indexing or weighting may be directly associated with the interpretive comment in the report. For example, the index may be provided as a subscript, superscript, parenthetical, or other text or visual indicator at the beginning or end of the interpretive comment. The index may also be represented in the display of all or a part of the interpretive comment (e.g., changing the color of the interpretive comment, the font, the size, etc.).

Indexing values for all or a subset of the interpretive comments may be generated manually or otherwise. An indexing value may be assigned based on a formula that weighs the reproducibility of the association, the size of the study supporting the interpretive comment, the type of study supporting the interpretive comment, the publication status of the study (which may include the source, e.g., journal, etc., of the study), a metric of how accepted the association is to those of skill in the art, and the presence of contradictory findings.

The report here also included other patient-specific information such as a patient identifier such as name, address, and/or a patient ID etc. The report also indicated the source of the genotyping assay results, including the sample type, ordering clinician, receive date, any notes/comments relating to the assay etc.

Lastly, in this specific example, the report included a summary of the results section that briefly summarizing the results of each biomarker test as well as the associated interpretive information and confidence index. In this example, the information summarized for each biomarker included: the biomarker tested (e.g., the SNP for SLC6A4), a description of what the biomarker is (“Serotonin Transporter”), an indicator of the result (“S/S”), and a brief description of the physiological significance of the test results. An image of the brain region implicated by the biomarker test result was also be shown.

While the reports, methods of generating them, systems, and methods for using them, have been described in some detail in U.S. Pat. No. 8,355,927, such illustration and example is for purposes of clarity of understanding only. It will be readily apparent to those of ordinary skill in the art in light of the teachings herein that certain changes and modifications may be made thereto without departing from the spirit and scope of the disclosure.

### VIII. Example System Embodiments

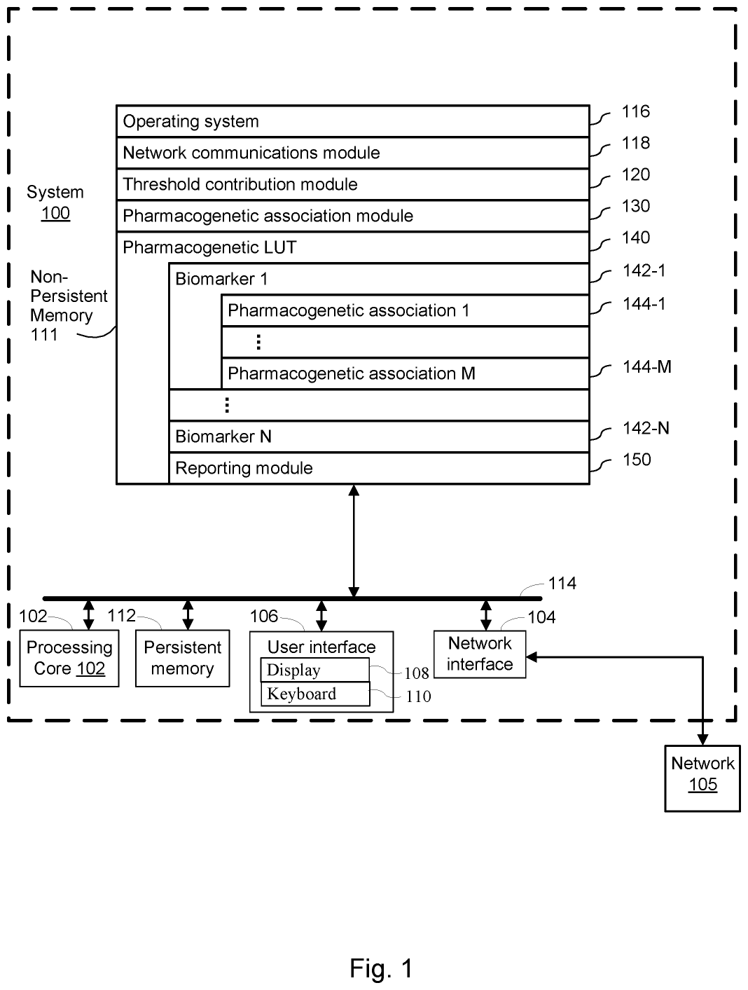

Details of an example system are described in relation to FIG. 1. FIG. 1 is a block diagram illustrating a system 100 for associating the allele status determined for the plurality of genomic loci with one or more recommendations for the treatment of a neuropsychiatric disorder and generating a patient-specific report comprising the one or more recommendations for the treatment of the neuropsychiatric disorder, in accordance with some implementations. Device 100, in some implementations, includes one or more processing units CPU(s) 102 (also referred to as processors or processing cores), one or more network interfaces 104, a user interface 106, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the system 100 with other devices and/or a communication network 105;
a threshold contribution module 120 for determining whether fluorescent signals corresponding to a variant allele meet a threshold contribution in the allele determination assay;
a pharmacogenetic association module 130 for associating variant alleles detected in the genotyping assay with recommendations for the management of one or more neuropsychiatric disorder, e.g., using a LUT 140 with pharmacogenetic associations 144 associated with each variant allele 142 detectable by the assay; and
a reporting module 150 for generating a patient-specific report comprising the one or more recommendations for the treatment of the neuropsychiatric disorder.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

Although FIG. 1 depicts a “system 100,” the figure is intended more as functional description of the various features, which may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules may be in persistent memory 112.

### EXAMPLES

### Example 1—Multiplexing Allele Discrimination Assay for SNPs rs5030863, rs28371685, and rs5030867

Multiplexed SNP assays were performed using 95 DNA samples plus 1 negative control. Each of these samples was added to respective multiplexed assay mixes containing fluorescent-labeled probes that are able to be detected by the QuantStudio 7 Flex instrument. Samples were then tested in singleton on a multi-well plate. Data analysis was performed using TaqMan® Genotyper software. Any genetic variant detected in a sample were followed up by singleplex SNP testing to identify the specific SNP variant.

Ninety-five DNA samples from various test subjects were collected using the Bucca Swabs technology. Each isolated DNA sample was diluted, aliquoted, and mixed with TaqMan® PCR reagents and three pairs of fluorescent-labeled oligonucleotide probes. Then, each of the 96 mixtures (including one mixture for negative control) was transferred into a single reaction well on a 384-well plate for QuantStudio 7. PCR amplification was performed. Fluorescent signal for each well was collected and quantified using two distinct fluorescence detection channels.

The oligonucleotide probe pairs were specifically designed to examine 3 genomic loci corresponding to SNPs rs5030863, rs28371685, and rs5030867 (Table 3). For each respective loci, one of the pairing probes were configured to recognize and anneal to a complementary DNA sequence containing the most prevalent wild-type (WT) SNP during the PCR process. Then, a 6-carboxyfluorescein (FAM) fluorophore at the 5′-end of the probe would be cleaved away by a Taq polymerase and released from a 5-carboxytetramethylrhodamine quencher at its 3′-end, resulting in significant fluorescence emission in the detection channel for FAM. The other pairing probe for the same loci recognizes a complementary DNA sequence containing the allelic variant (VAR) SNP. Similarly, this causes a 2′-chloro-7′phenyl-1,4-dichloro-6-carboxyfluorescein (VIC) fluorophore to be released from a 5-carboxytetramethylrhodamine quencher at the 3′-end of the probe, resulting in significant fluorescence emission in the detection channel for VIC.

TABLE 3
Genomic DNA Sequences Surrounding Targeted Loci

| NCBI SNP | | SEQ ID |
|---|---|---|
| Reference | Target and Surrounding Sequence | NO: |
| rs5030863 | GGCTGAACACGTCCCCGAAGCGGCGCCGCAA | 1 |
| | [G/C]TGCAGAGGGAGGGTCAGGGCCTCTTGTC | |
| rs28371685 | GATTGAACGTGTGATTGGCAGAAAC | 2 |
| | [T/C]GGAGCCCCTGCATGCAAGACAGGAG | |
| rs5030867 | GATGGGCTCACGCTGCACATCCGGA | 3 |
| | [G/T]GTAGGATCATGAGCAGGAGGCCCCA | |

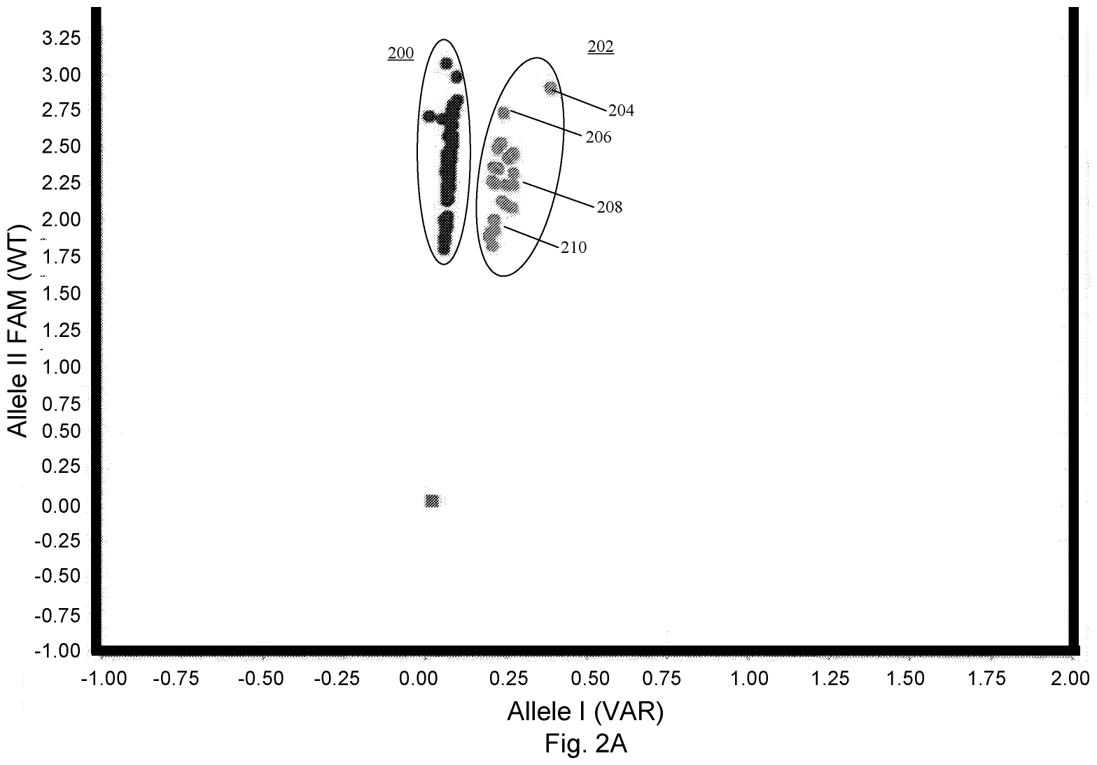

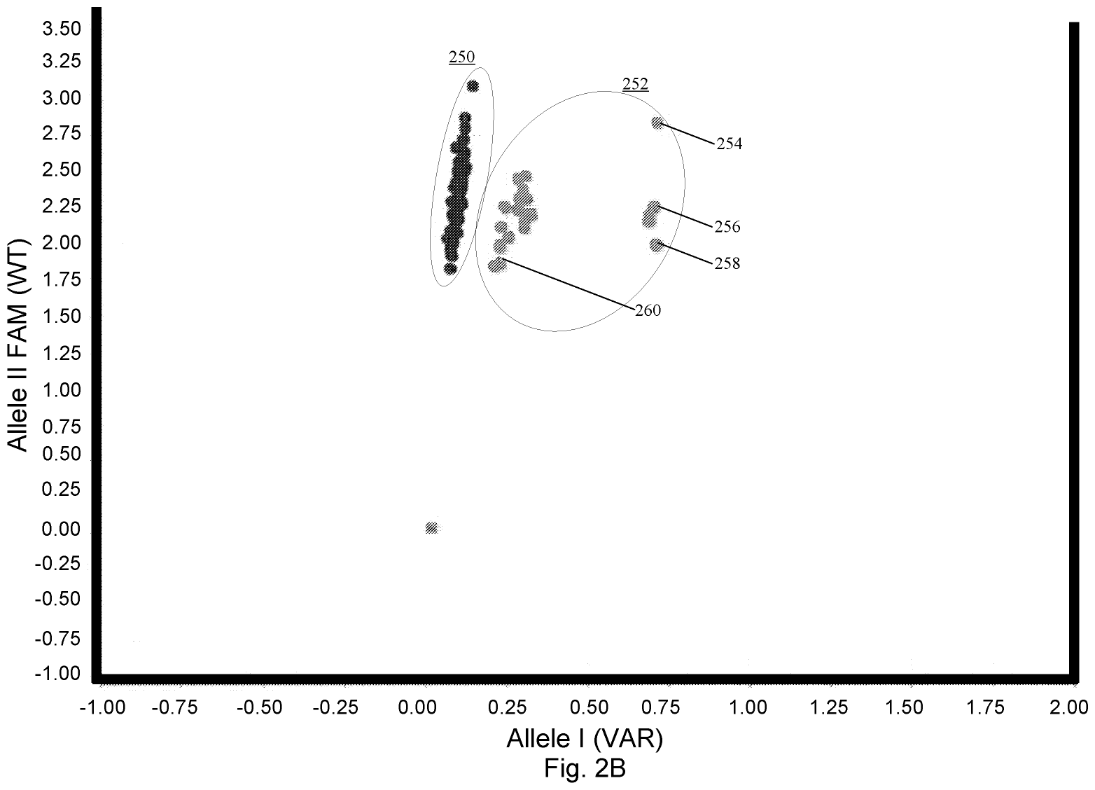

The testing result for each DNA sample was then plotted in aggregate as a function of the emission intensities from FAM and VIC, respectively (FIG. 2A). Based on the results, the samples can be classified into two groups: groups 200 and 202. DNA samples in both groups show substantial fluorescent signals from FAM in the range of about 1.75-3.25, indicating the presence of at least one WT allele in at least one of the three loci corresponding to rs5030863, rs28371685, and rs5030867. The finding of one or more WT alleles in these genomic loci is consistent with the high prevalence of the WT alleles in populations. By contrast, a majority of the samples show no VIC emission, indicating the absence of any VAR SNP at these loci. This finding is again consistent with the rarity of the VAR SNPs.

Notably, group 202 represents a substantial number of the DNA samples with distinguishable VIC emission signals at about 0.25. This distribution indicates the presence in these particular samples of at least one or more VAR SNPs across the tested loci. Next, each of these DNA samples would be selected and undergo single SNP test for each loci separately to determine their SNP genotypes. For example, the DNA samples corresponding to data points 204, 206, 208, and 210 were selected. Each of these DNAs was transferred to a respective reaction vessel on a multi-well plate, and then mixed with TaqMan® PCR reagents and the pairing TaqMan® probes configured for the corresponding loci for rs5030863. Similar PCR amplification as described above was performed and the fluorescent signal of each sample was measured to determine its SNP status at this particular loci. The same test was repeated for the corresponding loci for rs28371685, and rs5030867 as well, until the SNP status of all three loci were determined for each sample.

### Example 2—Multiplexing Allele Discrimination Assay for SNPs rs17884712, rs72552267, and rs72558187

Multiplexed SNP assays were performed using 95 DNA samples plus 1 negative control. Each of these samples was added to respective multiplexed assay mixes containing fluorescent-labeled probes that are able to be detected by the QuantStudio 7 Flex instrument. Samples were then tested in singleton on a multi-well plate. Data analysis was performed using TaqMan® Genotyper software. Any genetic variant detected in a sample were followed up by singleplex SNP testing to identify the specific SNP variant.

Ninety-five DNA samples from various test subjects were collected using the Bucca Swabs technology. Each isolated DNA sample was diluted, aliquoted, and mixed with TaqMan® PCR reagents and three pairs of fluorescent-labeled oligonucleotide probes. Then, each of the 96 mixtures (including one mixture for negative control) was transferred into a single reaction well on a 384-well plate for QuantStudio 7. PCR amplification was performed. Fluorescent signal for each well was collected and quantified using two distinct fluorescence detection channels.

The oligonucleotide probes were specifically designed to examine 3 genomic loci corresponding to SNPs rs17884712, rs72552267, and rs72558187 (Table 4). For each respective loci, one of the pairing probes were configured to recognize and anneal to a complementary DNA sequence containing the most prevalent wild-type (WT) SNP during the PCR process. Then, a 6-carboxyfluorescein (FAM) fluorophore at the 5′-end of the probe would be cleaved away by a Taq polymerase and released from a 5-carboxytetramethylrhodamine quencher at its 3′-end, resulting in significant fluorescence emission in the detection channel for FAM. The other pairing probe for the same loci recognizes a complementary DNA sequence containing the allelic variant (VAR) SNP. Similarly, this causes a 2′-chloro-7′phenyl-1,4-dichloro-6-carboxyfluorescein (VIC) fluorophore to be released from a 5-carboxytetramethylrhodamine quencher at the 3′-end of the probe, resulting in significant fluorescence emission in the detection channel for VIC.

TABLE 4
Genomic DNA Sequences Surrounding Targeted Loci

| | | SEQ |
|---|---|---|
| NCBI SNP | | ID |
| Reference | Target and Surrounding Sequence | NO: |
| rs17884712 | ATGGGGAAGAGGAGCATTGAGGACC[A/G] | 4 |
| | TGTTCAAGAGGAAGCCCGCTGCCTT | |
| rs72552267 | CGGCGTTTCTCCCTCATGACGCTGC[A/G] | 5 |
| | GAATTTTGGGATGGGGAAGAGGAGC | |
| rs72558187 | GCAGTGAAGGAAGCCCTGATTGATC[C/T] | 6 |
| | TGGAGAGGAGTTTTCTGGAAGAGGC | |

The testing result for each DNA sample was then plotted in aggregate as a function of the emission intensities from FAM and VIC, respectively (FIG. 2B). Based on the results, the samples can be classified into two groups: groups 250 and 252. DNA samples in both groups show substantial fluorescent signals from FAM in the range of about 1.75-3.25, indicating the presence of at least one WT allele in at least one of the three loci corresponding to rs17884712, rs72552267, and rs72558187. The finding of one or more WT alleles in these genomic loci is consistent with the high prevalence of the WT alleles in populations. By contrast, a majority of the samples show no VIC emission, indicating the absence of any VAR SNP at these loci. This finding is again consistent with the rarity of the VAR SNPs.

Notably, group 252 represents a substantial number of the DNA samples show distinguishable VIC emission signals ranging from about 0.25 to 0.75. This distribution indicates the presence in these particular samples of at least one or more VAR SNPs across the tested loci. Next, each of these DNA samples would be selected and undergo single SNP test for each loci separately to determine their SNP genotypes. For example, the DNA samples corresponding to data points 254, 256, 258, and 260 were selected. Each of these DNAs was transferred to a respective reaction vessel on a multi-well plate, and then mixed with TaqMan® PCR reagents and the pairing TaqMan® probes configured for the corresponding loci for rs17884712. Similar PCR amplification as described above was performed and the fluorescent signal of each sample was measured to determine its SNP status at this particular loci. The same test was repeated for the corresponding loci for rs72552267, and rs72558187 as well, until the SNP status of all three loci were determined for each sample.

### Example 3—Multiplexing Allele Discrimination Assay for SNPs rs5030862 and rs56337013

Multiplexed SNP assays were performed using 95 DNA samples plus 1 negative control. Each of these samples was added to respective multiplexed assay mixes containing fluorescent-labeled probes that are able to be detected by the QuantStudio 7 Flex instrument. Samples were tested in singleton on a multi-well plate. Analysis is performed using TaqMan® Genotyper software. Any genetic variant detected in a sample were followed up by single SNP testing to identify the specific SNP variant.

Ninety-five DNA samples from various test subjects were collected using the Bucca Swabs technology. Each isolated DNA sample was diluted, aliquoted, and mixed with TaqMan® PCR reagents and three pairs of fluorescent-labeled oligonucleotide probes. Then, each of the 96 mixtures (including one mixture for negative control) was transferred into a single reaction well on a 384-well plate for QuantStudio 7. PCR amplification was performed. Fluorescent signal for each well was collected and quantified using two distinct fluorescence detection channels.

The oligonucleotide probes were specifically designed to sample 2 genomic loci corresponding to SNPs rs5030862 and rs56337013 (Table 5). For each respective loci, one of the pairing probes were configured to recognize and anneal to a complementary DNA sequence containing the most prevalent wild-type (WT) SNP during the PCR process. Then, a 2′-chloro-7′phenyl-1,4-dichloro-6-carboxyfluorescein (VIC) fluorophore at the 5′-end of the probe would be cleaved away by a Taq polymerase and released from a 5-carboxytetramethylrhodamine quencher at its 3′-end, resulting in significant fluorescence emission in the detection channel for VIC. The other pairing probe for the same loci recognizes a complementary DNA sequence containing the allelic variant (VAR) SNP. Similarly, this causes a 6-carboxyfluorescein (FAM) fluorophore to be released from a 5-carboxytetramethylrhodamine quencher at the 3′-end of the probe, resulting in significant fluorescence emission in the detection channel for FAM.

TABLE 5
Genomic DNA Sequences Surrounding Targeted Loci

| NCBI SNP | | SEQ |
|---|---|---|
| Reference | Target and Surrounding Sequence | ID NO: |
| rs5030862 | TCCACATGCAGCAGGTTGCCCAGCC[C/T] | 7 |
| | GGGCAGTGGCAGGGGGCCTGGTGGG | |
| rs56337013 | CCTATGTTTGTTATTTTCAGGAAAA[C/T] | 8 |
| | GGATTTGTGTGGGAGAGGGCCTGGC | |

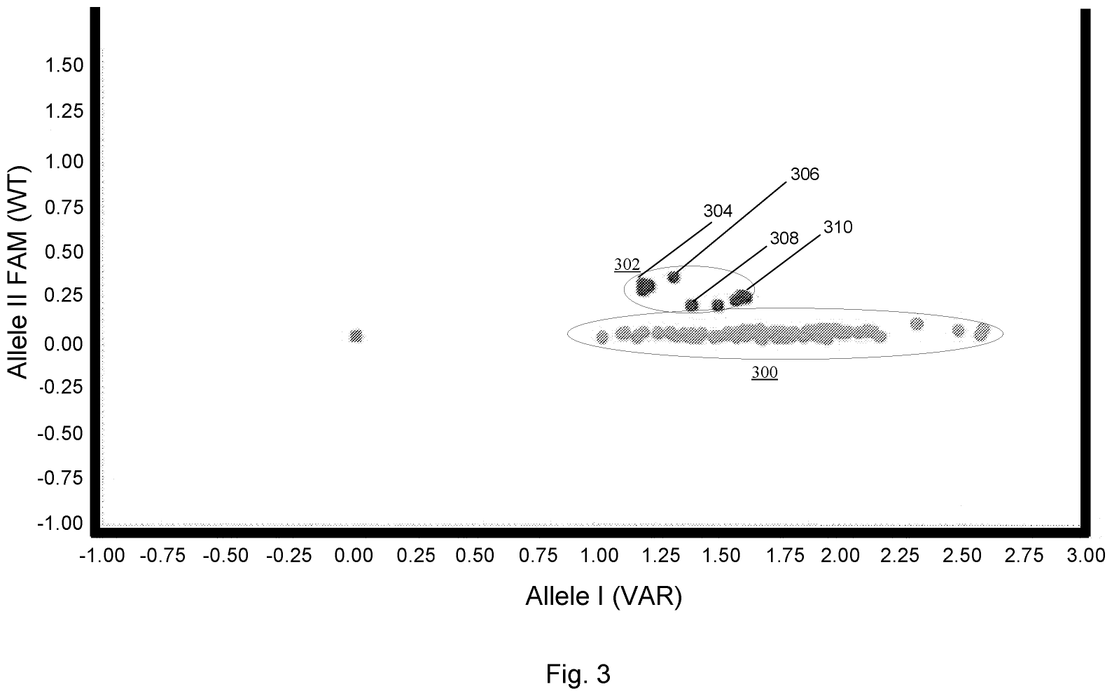

The testing result for each DNA sample was then plotted in aggregate as a function of the emission intensities from VIC and FAM, respectively (FIG. 3). Based on the results, the samples can be classified into two groups: groups 300 and 302. DNA samples in both groups show substantial fluorescent signals from VIC ranging from about 1.0 to 2.75, indicating the presence of at least one WT allele in at least one of the two loci corresponding to rs5030862 and rs56337013. The finding of one or more WT alleles in these genomic loci is consistent with the high prevalence of the WT alleles in populations. By contrast, a substantial majority of the samples show no FAM emission, indicating the absence of any VAR SNP at either loci. This finding is again consistent with the rarity of the VAR SNPs.

Notably, group 302 represents a good number of the DNA samples with distinguishable FAM emission signals at about 0.25. This distribution indicates the presence in these particular samples of at least one or more VAR SNPs across the tested loci. Next, each of these DNA samples would be selected and undergo single SNP test for each loci separately to determine their SNP genotypes. For example, the DNA samples corresponding to data points 304, 306, 308, and 310 were selected. Each of these DNAs was transferred to a respective reaction vessel on a multi-well plate, and then mixed with TaqMan® PCR reagents and the pairing TaqMan® probes configured for the corresponding loci for rs5030862. Similar PCR amplification as described above was performed and the fluorescent signal of each sample was measured to determine its SNP status at this particular loci. The same test was repeated for the corresponding loci for rs56337013 so the SNP status of both loci were determined for each sample.

### Example 4—Multiplexing Genotyping Assay Reduces the Number of Tests Required

Frequency (i.e., probability) of a SNP is defined as the relative ratio of the number of occurrence of the SNP within a population to the overall size of the population. Table 6 below summarizes the corresponding frequency that each SNP described in Examples 1, 2, and 3 is found within the human population (data source: GenomAD_exome).

TABLE 6
SNP Frequency

| NCBI SNP | SNP |
|---|---|
| Reference | Frequency |
| rs5030863 | 0.00011 |
| (rs201377835) | |
| rs28371685 | 0.00393 |
| rs5030867 | 0.00118 |
| rs17884712 | 0.00124 |
| rs72552267 | 0.00031 |
| rs72558187 | 0.00011 |
| rs5030862 | 0.00007 |
| rs56337013 | 0.00001 |

As described for many of the embodiments provided herein, the first round of a multiplex genotyping assay screens a plurality of genomic loci of a test subject in a single reaction, to determine if any of these loci carry a SNP associated with a relevant pharmacogenetic effect. When no SNPs are present at any of the tested loci, testing is terminated and the result that the subject does not carry a pharmacogenetic allele at any of the tested loci is reported. However, when one or more SNPs are present in the sample, a second round of genotyping assays is triggered. In the second round, in contrast to the multiplexing assay, a series of single genotyping tests are performed to characterize the allele status of each respective loci, where each individual loci is being tested separately.

The average frequency of triggering the second round of assays is calculated using the following formula: f=1−(1−F₁)²×(1−F₂)²×(1−F₃)²× . . . ×(1−Fₙ)². Here, f equals the overall frequency of at least one SNP being present in the genomic loci tested, and F₁ to Fₙ are the respective frequency of each respective SNP in the human population. This equation can be used to determine the expected frequency with which additional testing will be required for any combination of SNPs detected together in a multiplexed assay.

For instance, in Example 1, three genomic loci corresponding to three SNPs: rs5030863, rs28371685, and rs5030867, were screened first in a single test (i.e., the first round). Based on the formula above, the frequency/probability of any of these SNPs being present in a subject is 0.0104=1−(1−0.00011)²×(1−0.00393)²×(1−0.00118)², or 1.04%. In other words, when screening the three genomic loci for any of the three SNPs above, there is a frequency of 0.0104, or probability of 1.04%, that the nucleic acid sample will need to undergo a second round of testing.

Accordingly, for every 1000 subjects tested according to the Example 2, it is expected that approximately ten subjects will have to undergo a second round of genotyping assays. Thus, for every 1000 subjects tested, it would be expected that about 1030 assays would need to be performed to determine the allele status at each of the three identified loci in all of the subjects (1000 multiplexed assays+1000 (total patients)×3 (secondary assays required per re-test)×0.0104 (frequency of re-testing)=1031). In comparison, using conventional, non-multiplexed genotyping assays to determine the allele status of the three genomic loci for the 1000 samples, each of the three loci in a sample must be tested separately, requiring 3000 genotyping reactions. Therefore, probabilistically, the multiplexing genotyping assays describe herein significantly reduces the number of tests required by approximately two-thirds.

Similarly, in Example 2, three genomic loci corresponding to three SNPs: rs17884712, rs72552267, and rs72558187, were screened first in a single test (i.e., the first round). Based on the formula above, the frequency/probability of any of these SNPs being present in a subject is 0.0033=1−(1−0.00124)²×(1−0.00031)²×(1−0.00011)², or 0.33%. In other words, when screening the three genomic loci for any of the three SNPs above, there is a frequency of 0.0033, or probability of 0.33%, that the nucleic acid sample will need to undergo a second round of testing.

Accordingly, for every 1000 subjects tested according to the Example 2, it is expected that approximately three subjects will have to undergo a second round of genotyping assays. Thus, for every 1000 subjects tested, it would be expected that about 1009 assays would need to be performed to determine the allele status at each of the three identified loci in all of the subjects (1000 multiplexed assays+1000 (total patients)×3 (secondary assays required per re-test)×0.0033 (frequency of re-testing)=1010). In comparison, using conventional, non-multiplexed genotyping assays to determine the allele status of the three genomic loci for the 1000 samples, each of the three loci in a sample must be tested separately, requiring 3000 genotyping reactions. Therefore, probabilistically, the multiplexing genotyping assays describe herein significantly reduces the number of tests required by approximately two-thirds.

Similarly, in Example 3, two genomic loci corresponding to two SNPs: rs5030862 and rs56337013, were screened first in a single test (i.e., the first round). Based on the formula above, the frequency/probability of any of these SNPs being present in a subject is 0.0002=1−(1−0.00007)²×(1−0.00001)² or 0.02%. In other words, when screening the two genomic loci for any of the two SNPs above, there is a frequency of 0.0002, or probability of 0.02%, that the nucleic acid sample will need to undergo a second round of testing.

Accordingly, for every 10,000 subjects tested according to the Example 3, it is expected that approximately two subjects will have to undergo a second round of genotyping assays. Thus, for every 10,000 subjects tested, it would be expected that about 10,004 assays would need to be performed to determine the allele status at each of the three identified loci in all of the subjects (10,000 multiplexed assays+10,000 (total patients)×2 (secondary assays required per re-test)×0.0002 (frequency of re-testing)=10,004). In comparison, using conventional, non-multiplexed genotyping assays to determine the allele status of the two genomic loci for the 10,000 samples, each of the three loci in a sample must be tested separately, requiring 20,000 genotyping reactions. Therefore, probabilistically, the multiplexing genotyping assays describe herein significantly reduces the number of tests required by approximately half.

If the three multiplexed reactions described in Examples 1-3 were used to screen all eight of the alleles for 10,000 patients, it would be expected that 10,300+10,100+10,002=30,402 total reactions would have to be performed. In comparison, using conventional, non-multiplexed genotyping assays to determine the allele status of the three genomic loci for the 1000 samples, each of the eight loci in a sample must be tested separately, requiring 8000 genotyping reactions. Therefore, use of the three multiplexing genotyping assays would significantly reduce the number of tests required by approximately 62%.

Finally, if all eight of the alleles tested in Examples 1-3 were screened together in multiplex assay as described herein, the frequency/probability of any of these SNPs being present in a subject would be 0.0138=1−(1−0.00011)²×(1−0.00393)²×(1−0.00118)²×(1−0.00124)²×(1−0.00031)²×(1−0.00011)²×(1−0.00007)²×(1−0.00001)², or 1.38%. In other words, when screening the three genomic loci for any of the eight SNPs above, there is a frequency of 0.0138, or probability of 1.38%, that the nucleic acid sample will need to undergo a second round of testing.

Accordingly, for every 1000 subjects tested, it would be expected that approximately fourteen subjects will have to be re-tested. Thus, for every 1000 subjects tested, it would be expected that about 1112 assays would need to be performed to determine the allele status at each of the three identified loci in all of the subjects (1000 multiplexed assays+1000 (total patients)×8 (secondary assays required per re-test)×0.0138 (frequency of re-testing)=1110). In comparison, using conventional, non-multiplexed genotyping assays to determine the allele status of the three genomic loci for the 1000 samples, each of the eight loci in a sample must be tested separately, requiring 8000 genotyping reactions. Therefore, probabilistically, the multiplexing genotyping assays describe herein significantly reduces the number of tests required by approximately 86%.

### CONCLUSION

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method for determining an allele status at a plurality of genomic loci in a subject, the method comprising:
a) amplifying, in a single in vitro reaction vessel, a first plurality of genomic loci, by polymerase chain reaction (PCR), from nucleic acids isolated from a sample obtained from the subject, in the presence of a plurality of fluorescently-labeled detection reagents, wherein the plurality of fluorescently-labeled detection reagents includes, for each respective genomic locus in the first plurality of genomic loci:
a first detection reagent that is specific for the presence of a first allele at the respective genomic locus, wherein the first allele is the most prevalent allele in a population of the species of the subject and the first detection reagent is labeled with a first fluorescent moiety, and
a second detection reagent that is specific for the presence of a second allele at the respective genomic locus, wherein the second allele is a minor allele in the population and the second detection reagent is labeled with a second fluorescent moiety that is distinguishable from the first fluorescent moiety;
b) during or after the amplifying a), detecting a fluorescent signal corresponding to the first fluorescent moiety and the second fluorescent moiety in the reaction vessel; and
c) responsive to the detecting b):
when the contribution of the second fluorescent moiety to the fluorescent signal detected in the reaction vessel does not satisfy a threshold contribution, reporting that the subject does not carry the second allele at any of the first plurality of genomic loci, and
when the contribution of the second fluorescent moiety to the fluorescent signal detected in the reaction vessel satisfies the threshold contribution:
performing a first plurality of secondary allele detection assays, wherein each secondary allele detection assay in the first plurality of secondary allele detection assays determines the allele status at one respective genomic locus in the first plurality of genomic loci, and
reporting the allele status at each of the first plurality of genomic loci based on the first plurality of secondary allele detection assays.

2. The method of claim 1, wherein the first plurality of genomic loci is at least three genomic loci.

3. The method of claim 1, wherein the species is human and the first plurality of genomic loci comprise at least three genomic loci, at least four genomic loci, at least five genomic loci, or at least 10 genomic loci in Table 1 and/or Table 2.

4. The method of claim 1 or 2, wherein the species is human.

5. The method of any one of claims 1-4, wherein the sample obtained from the subject comprises buccal cells, saliva, or blood.

6. The method of any one of claims 1-5, wherein, for each respective genomic locus in the first plurality of genomic loci, the frequency of the first allele in the population is at least 95%.

7. The method of any one of claims 1-6, wherein, for each respective genomic locus in the first plurality of genomic loci, the frequency of the second allele in the population is no more than 5%.

8. The method of any one of claims 1-7, wherein the combined frequency, in the population, of the second allele for each respective loci in the first plurality of loci is no more than 10%.

9. The method of any one of claims 1-8, wherein, for each respective genomic locus in the first plurality of genomic loci:
the first detection reagent comprises a first oligonucleotide labeled with a first matching pair of electronic energy transfer chromophores, wherein the sequence of the first oligonucleotide is complementary to the first allele at the respective genomic locus; and
the second detection reagent comprises a second oligonucleotide labeled with a second matching pair of electronic energy transfer chromophores, wherein the sequence of the second oligonucleotide is complementary to the second allele at the respective genomic locus.

10. The method of claim 9, wherein, for each respective genomic locus in the first plurality of genomic loci:
the first matching pair of electronic energy transfer chromophores consists of a first excitation chromophore and a first quenching chromophore for the first excitation chromophore; and
the second matching pair of electronic energy transfer chromophores consists of a second excitation chromophore and a second quenching chromophore for the second excitation chromophore.

11. The method of claim 10, wherein, for each respective genomic locus in the first plurality of genomic loci:
one of the first matching pair of electronic energy transfer chromophores or the second matching pair of electronic energy transfer chromophores consists of a 6-carboxyfluorescein excitation chromophore and a 5-carboxytetramethylrhodamine quenching chromophore; and
the other of the first matching pair of electronic energy transfer chromophores or the second matching pair of electronic energy transfer chromophores consists of a 2′-chloro-7′phenyl-1,4-dichloro-6-carboxy-fluorescein excitation chromophore and a 5-carboxytetramethylrhodamine quenching chromophore.

12. The method of any one of claims 1-11, wherein:
the contribution of the second fluorescent moiety to the fluorescent signal detected in the reaction vessel satisfies the threshold contribution when the contribution of the second fluorescent moiety to the fluorescent signal indicates that, for at least one respective genomic locus in the plurality of genomic loci, the first allele is not present in at least one half of the nucleic acids encompassing the respective loci; and
the contribution of the second fluorescent moiety to the fluorescent signal detected in the reaction vessel does not satisfy the threshold contribution when the contribution of the second fluorescent moiety to the fluorescent signal indicates that, for each respective genomic locus in the plurality of genomic loci, the first allele is present in more than one half of the nucleic acids encompassing the respective loci.

13. The method of any one of claims 1-12, wherein the first plurality of genomic loci comprises the human alleles corresponding to the SNPs rs5030863, rs28371685, and rs5030867.

14. The method of claim 13, further comprising administering, to the subject, a low dose of a pharmaceutical agent that is metabolized by cytochrome P450 2D6 (CYP2D6) when the subject is determined to carry the rs5030863 SNP.

15. The method of claim 13 or 14, further comprising administering, to the subject, a low dose of a pharmaceutical agent that is metabolized by cytochrome P450 2C9 (CYP2C9) when the subject is determined to carry the rs28371685 SNP.

16. The method of any one of claims 13-15, further comprising administering, to the subject, a low dose of an antipsychotics when the subject is determined to carry the rs5030867 SNP.

17. The method of any one of claims 1-12, wherein the first plurality of genomic loci comprises the human alleles corresponding to the SNPs rs17884712, rs72552267, and rs72558187.

18. The method of claim 17, further comprising administering, to the subject, a low dose of a pharmaceutical agent that is metabolized by cytochrome P450 2C19 (CYP2C19) when the subject is determined to carry the rs17884712 SNP.

19. The method of claim 17 or 18, further comprising administering, to the subject, a low dose of a pharmaceutical agent that is metabolized by cytochrome P450 2C19 (CYP2C19) when the subject is determined to carry the rs72552267 SNP.

20. The method of any one of claims 17-19, further comprising administering, to the subject, a low dose of antiepileptic drug (AED) that is metabolized by cytochrome P450 2C9 (CYP2C9) when the subject is determined to carry the rs72558187 SNP.

21. The method of any one of claims 1-12, wherein the first plurality of genomic loci comprises the human alleles corresponding to the SNPs rs5030862 and rs56337013.

22. The method of claim 21, further comprising administering, to the subject, a low dose of a pharmaceutical agent that is metabolized by cytochrome P450 2D6 (CYP2D6) when the subject is determined to carry the rs5030862 SNP.

23. The method of claim 21 or 22, further comprising administering, to the subject, a low dose of a pharmaceutical agent that is metabolized by cytochrome P450 2C19 (CYP2C19) when the subject is determined to carry the rs56337013 SNP.

24. The method of any one of claims 1-23, wherein:
the amplifying a) further comprises:
amplifying, in the single in vitro reaction vessel, a second plurality of genomic loci, by polymerase chain reaction (PCR), from the nucleic acids isolated from the sample obtained from the subject, in the presence of the plurality of fluorescently-labeled detection reagents, wherein the plurality of fluorescently-labeled detection reagents includes, for each respective genomic locus in the second plurality of genomic loci:
a first detection reagent that is specific for the presence of a first allele at the respective genomic locus, wherein the first allele is the most prevalent allele in a population of the species of the subject and the first detection reagent is labeled with a third fluorescent moiety that is distinguishable from the first fluorescent moiety and the second fluorescent moiety, and
a second detection reagent that is specific for the presence of a second allele at the respective genomic locus, wherein the second allele is a minor allele in the population and the second detection reagent is labeled with a fourth fluorescent moiety that is distinguishable from the first fluorescent moiety, the second fluorescent moiety, and the third fluorescent moiety;
the detecting b) further comprises:
detecting a fluorescent signal corresponding to the third fluorescent moiety and the fourth fluorescent moiety in the reaction vessel; and
responsive to the detecting b), the method comprises:
when the contribution of the fourth fluorescent moiety to the fluorescent signal detected in the reaction vessel does not satisfy a threshold contribution, reporting that the subject does not carry the second allele at any of the second plurality of genomic loci, and
when the contribution of the fourth fluorescent moiety to the fluorescent signal detected in the reaction vessel satisfies the threshold contribution:
performing a second plurality of secondary allele detection assays, wherein each secondary allele detection assay in the second plurality of secondary allele detection assays determines the allele status at one respective genomic locus in the second plurality of genomic loci, and
reporting the allele status at each of the second plurality of genomic loci based on the second plurality of secondary allele detection assays.

25. A method for performing a high throughput genotyping assay, the method comprising:
a) dispensing, into each respective well in a first plurality of wells in a multiwell plate, in accordance with one or more template plate definitions associated with the high throughput genotyping assay, a respective template nucleic acid preparation, reagents for amplifying a first plurality of genomic loci, and a first plurality of fluorescently-labeled detection reagents, wherein:
the respective template nucleic acid preparation dispensed into each respective well is prepared from a respective biological sample obtained from a different test subject in a plurality of test subjects, and
the first plurality of fluorescently-labeled detection reagents includes, for each respective genomic locus in the first plurality of genomic loci:
a first detection reagent that is specific for the presence of a first allele at the respective genomic locus, wherein the first allele is the most prevalent allele in a population of the species of the subject and the first detection reagent is labeled with a first fluorescent moiety, and
a second detection reagent that is specific for the presence of a second allele at the respective genomic locus, wherein the second allele is a minor allele in the population and the second detection reagent is labeled with a second fluorescent moiety that is distinguishable from the first fluorescent moiety;
b) amplifying, after the dispensing a), the first plurality of genomic loci in each respective well;
c) detecting, during or after the amplifying b), in each respective well, a fluorescent signal corresponding to the first fluorescent moiety and the second fluorescent moiety in the reaction vessel; and
d) responsive to the detecting c), for each respective well:
when the contribution of the second fluorescent moiety to the fluorescent signal detected in the respective well does not satisfy a threshold contribution, reporting that the corresponding subject in the plurality of subjects does not carry the second allele at any of the first plurality of genomic loci, and
when the contribution of the second fluorescent moiety to the fluorescent signal detected in the respective well satisfies the threshold contribution:
performing a first plurality of secondary allele detection assays using a template nucleic acid preparation from the corresponding subject in the plurality of subjects, wherein each secondary allele detection assay in the first plurality of secondary allele detection assays determines the allele status at one respective genomic locus in the first plurality of genomic loci, and
reporting the allele status of the corresponding subject at each of the first plurality of genomic loci based on the first plurality of secondary allele detection assays.

26. The method of claim 25, wherein, for each respective genomic locus in the first plurality of genomic loci, the frequency of the first allele in the population is at least 95%.

27. The method of any one of claim 25 or 26, wherein, for each respective genomic locus in the first plurality of genomic loci, the frequency of the second allele in the population is no more than 5%.

28. The method of any one of claims 25-27, wherein the combined frequency, in the population, of the second allele for each respective loci in the first plurality of loci is no more than 10%.

29. The method of any one of claims 25-28, wherein the dispensing a) is performed by an automated liquid handler.

30. The method of any one of claims 25-28, further comprising:
a1) dispensing, into each respective well in a second plurality of wells in a multiwell plate, in accordance with the one or more template plate definitions associated with the high throughput genotyping assay, a respective template nucleic acid preparation, reagents for amplifying a second plurality of genomic loci, and a second plurality of fluorescently-labeled detection reagents, wherein:
the respective template nucleic acid preparation dispensed into each respective well is prepared from a respective biological sample obtained from a different test subject in the plurality of test subjects, and
the second plurality of fluorescently-labeled detection reagents includes, for each respective genomic locus in the second plurality of genomic loci:
a third detection reagent that is specific for the presence of a first allele at the respective genomic locus, wherein the first allele is the most prevalent allele in a population of the species of the subject and the third detection reagent is labeled with a third fluorescent moiety, and
a fourth detection reagent that is specific for the presence of a second allele at the respective genomic locus, wherein the second allele is a minor allele in the population and the fourth detection reagent is labeled with a second fluorescent moiety that is distinguishable from the third fluorescent moiety;
b1) after the dispensing a1), amplifying the second plurality of genomic loci in each respective well;
c1) during or after the amplifying b1), detecting, in each respective well, a fluorescent signal corresponding to the third fluorescent moiety and the fourth fluorescent moiety; and
d1) responsive to the detecting c1), for each respective well:
when the contribution of the fourth fluorescent moiety to the fluorescent signal detected in the respective well does not satisfy a threshold contribution, reporting that the corresponding subject in the plurality of subjects does not carry the second allele at any of the second plurality of genomic loci, and
when the contribution of the fourth fluorescent moiety to the fluorescent signal detected in the respective well satisfies the threshold contribution:
performing a second plurality of secondary allele detection assays using a template nucleic acid preparation from the corresponding subject in the plurality of subjects, wherein each secondary allele detection assay in the second plurality of secondary allele detection assays determines the allele status at one respective genomic locus in the second plurality of genomic loci, and
reporting the allele status of the corresponding subject at each of the second plurality of genomic loci based on the second plurality of secondary allele detection assays.

31. The method of claim 30, wherein the first plurality of wells and the second plurality of wells are in the same multiwell plate.

32. The method of claim 30, wherein the first plurality of wells and the second plurality of wells are in different multiwell plates.

33. The method of any one of claims 25-32, wherein the reporting further includes, when it is determined that a respective subject carries a minor allele at a respective genomic locus in the plurality of genomic loci, reporting a warning, precaution, or drug interaction for a pharmaceutical agent associated with the minor allele of the respective loci.

34. The method of any one of claims 25-33, wherein the a) dispensing, b) amplifying, and c) detecting are performed within six hours.

35. The method of any one of claims 25-33, wherein the a) dispensing, b) amplifying, and c) detecting are performed within four hours.

36. The method of any one of claims 25-34, wherein the plurality of genomic loci comprises at least three genomic loci, at least four genomic loci, at least five genomic loci, or at least 10 genomic loci in Table 1 and/or Table 2

37. The method of any one of claims 25-34, wherein the plurality of genomic loci consists of between two and twenty genomic loci in Table 1 and/or Table 2.

38. A method for providing guidance for the treatment of a neuropsychiatric disorder in a subject, the method comprising:
a) determining the allele status for a plurality of genomic loci, wherein each respective loci in the plurality of loci is associated with a therapeutic efficacy of at least one therapy for a neuropsychiatric disorder, the determining comprising:
i) amplifying, in a first single in vitro reaction vessel, a first set of two or more genomic loci in the plurality of genomic loci, by polymerase chain reaction (PCR), from nucleic acids isolated from a sample obtained from the subject, in the presence of a plurality of fluorescently-labeled detection reagents, wherein the plurality of fluorescently-labeled detection reagents includes, for each respective genomic locus in the two or more genomic loci:
a first detection reagent that is specific for the presence of a first allele at the respective genomic locus, wherein the first allele is the most prevalent allele in a population of the species of the subject and the first detection reagent is labeled with a first fluorescent moiety, and
a second detection reagent that is specific for the presence of a second allele at the respective genomic locus, wherein the second allele is a minor allele in the population and the second detection reagent is labeled with a second fluorescent moiety that is distinguishable from the first fluorescent moiety;
ii) during or after the amplifying i), detecting a fluorescent signal corresponding to the first fluorescent moiety and the second fluorescent moiety in the first single reaction vessel; and
iii) responsive to the detecting ii):
when the contribution of the second fluorescent moiety to the fluorescent signal detected in the reaction vessel does not satisfy a threshold contribution, determining that the subject is homozygous for the first allele at each respective loci in the first set of two or more genomic loci, and
when the contribution of the second fluorescent moiety to the fluorescent signal detected in the reaction vessel satisfies the threshold contribution, performing a first plurality of secondary allele detection assays, wherein each secondary allele detection assay in the first plurality of secondary allele detection assays determines the allele status at one respective genomic locus in the first set of two or more genomic loci, thereby determining the allele status at each respective loci in the first set of two or more genomic loci;
b) associating the allele status determined for the plurality of genomic loci with one or more recommendations for the treatment of the neuropsychiatric disorder; and
c) generating a patient-specific report comprising the one or more recommendations for the treatment of the neuropsychiatric disorder.

39. The method of claim 38, wherein the plurality of genomic loci comprises one or more genomic loci corresponding to a SNP selected from the group consisting of rs7997012, rs3813929, rs1045642, rs2032583, rs1800544, rs10994336, rs6265, rs1006737, rs4680, rs2470890 (CYP1A2*1B), rs2069514, rs35694136, rs2069526 (CYP1A2*1E), rs762551, rs12720461 (CYP1A2*1K), rs2069526 (CYP1A2*1K), rs72547513, rs2279343 (CYP2B6*4), rs3211371, rs3745274 (CYP2B6*6), rs2279343 (CYP2B6*6), rs4244285, rs17878459 (CYP2C19*2B), rs4986893 (CYP2C19*3), rs57081121 (CYP2C19*3), rs28399504, rs56337013, rs72552267, rs72558186, rs41291556, rs17884712, rs6413438, rs12248560, rs12769205 (CYP2C19*35), rs3758581 (CYP2C19*35), rs1799853, rs1057910, rs56165452, rs28371686, rs9332131, rs7900194, rs28371685, rs72558187, rs7900194 (CYP2C9*27), rs16947 (CYP2D6*2), rs1135840 (CYP2D6*2), rs1135824 (CYP2D6*3), rs35742686 (CYP2D6*3), rs3892097, rs5030655, rs5030867, rs5030865, rs5030656, rs1065852, rs5030863, rs5030862, rs5030865, rs774671100, rs28371706 (CYP2D6*17), rs16947 (CYP2D6*17), rs61736512 (CYP2D6*29), rs1058164 (CYP2D6*29), rs16947 (CYP2D6*29), rs59421388 (CYP2D6*29), rs1135840 (CYP2D6*29), rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023.

40. The method of claim 38, wherein the plurality of genomic loci comprises at least the genomic loci corresponding to SNPs rs7997012, rs3813929, rs1045642, rs2032583, rs1800544, rs10994336, rs6265, rs1006737, rs4680, rs2470890 (CYP1A2*1B), rs2069514, rs35694136, rs2069526 (CYP1A2*1E), rs762551, rs12720461 (CYP1A2*1K), rs2069526 (CYP1A2*1K), rs72547513, rs2279343 (CYP2B6*4), rs3211371, rs3745274 (CYP2B6*6), rs2279343 (CYP2B6*6), rs4244285, rs17878459 (CYP2C19*2B), rs4986893 (CYP2C19*3), rs57081121 (CYP2C19*3), rs28399504, rs56337013, rs72552267, rs72558186, rs41291556, rs17884712, rs6413438, rs12248560, rs12769205 (CYP2C19*35), rs3758581 (CYP2C19*35), rs1799853, rs1057910, rs56165452, rs28371686, rs9332131, rs7900194, rs28371685, rs72558187, rs7900194 (CYP2C9*27), rs16947 (CYP2D6*2), rs1135840 (CYP2D6*2), rs1135824 (CYP2D6*3), rs35742686 (CYP2D6*3), rs3892097, rs5030655, rs5030867, rs5030865, rs5030656, rs1065852, rs5030863, rs5030862, rs5030865, rs774671100, rs28371706 (CYP2D6*17), rs16947 (CYP2D6*17), rs61736512 (CYP2D6*29), rs1058164 (CYP2D6*29), rs16947 (CYP2D6*29), rs59421388 (CYP2D6*29), rs1135840 (CYP2D6*29), rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023.

41. The method of any one of claims 38-40, wherein the first set of two or more genomic loci comprises one of:
the human alleles corresponding to the SNPs rs5030863, rs28371685, and rs5030867,
the human alleles corresponding to the SNPs rs17884712, rs72552267, and rs72558187, or
the human alleles corresponding to the SNPs rs5030862 and rs56337013.

42. The method of any one of claims 38-41, wherein the determining a) further comprises:
iv) amplifying, in a second single in vitro reaction vessel, a second set of two or more genomic loci in the plurality of genomic loci, by polymerase chain reaction (PCR), from nucleic acids isolated from a sample obtained from the subject, in the presence of a plurality of fluorescently-labeled detection reagents, wherein the plurality of fluorescently-labeled detection reagents includes, for each respective genomic locus in the second set of two or more genomic loci:
a third detection reagent that is specific for the presence of a first allele at the respective genomic locus, wherein the first allele is the most prevalent allele in a population of the species of the subject and the third detection reagent is labeled with a third fluorescent moiety, and
a fourth detection reagent that is specific for the presence of a second allele at the respective genomic locus, wherein the second allele is a minor allele in the population and the fourth detection reagent is labeled with a fourth fluorescent moiety that is distinguishable from the third fluorescent moiety
v) during or after the amplifying iv), detecting a fluorescent signal corresponding to the third fluorescent moiety and the fourth fluorescent moiety in the second single reaction vessel; and
vi) responsive to the detecting v):
when the contribution of the fourth fluorescent moiety to the fluorescent signal detected in the reaction vessel does not satisfy a threshold contribution, determining that the subject is homozygous for the first allele at each respective loci in the second set of two or more loci, and
when the contribution of the fourth fluorescent moiety to the fluorescent signal detected in the reaction vessel satisfies the threshold contribution, performing a second plurality of secondary allele detection assays, wherein each secondary allele detection assay in the second plurality of secondary allele detection assays determines the allele status at one respective genomic locus in the second set of two or more genomic loci, thereby determining the allele status at each respective loci in the second set of two or more genomic loci.

43. The method of claim 42, wherein the second set of two or more genomic loci comprises one of:
the human alleles corresponding to the SNPs rs5030863, rs28371685, and rs5030867,
the human alleles corresponding to the SNPs rs17884712, rs72552267, and rs72558187, or
the human alleles corresponding to the SNPs rs5030862 and rs56337013.

44. The method of claim 42 or 43, wherein the determining a) further comprises:
vii) amplifying, in a third single in vitro reaction vessel, a third set of two or more genomic loci in the plurality of genomic loci, by polymerase chain reaction (PCR), from nucleic acids isolated from a sample obtained from the subject, in the presence of a plurality of fluorescently-labeled detection reagents, wherein the plurality of fluorescently-labeled detection reagents includes, for each respective genomic locus in the third set of two or more genomic loci:
a fifth detection reagent that is specific for the presence of a first allele at the respective genomic locus, wherein the first allele is the most prevalent allele in a population of the species of the subject and the fifth detection reagent is labeled with a third fluorescent moiety, and
a sixth detection reagent that is specific for the presence of a second allele at the respective genomic locus, wherein the second allele is a minor allele in the population and the sixth detection reagent is labeled with a sixth fluorescent moiety that is distinguishable from the fifth fluorescent moiety
viii) during or after the amplifying vii), detecting a fluorescent signal corresponding to the fifth fluorescent moiety and the sixth fluorescent moiety in the third single reaction vessel; and
ix) responsive to the detecting viii):
when the contribution of the sixth fluorescent moiety to the fluorescent signal detected in the reaction vessel does not satisfy a threshold contribution, determining that the subject is homozygous for the first allele at each respective loci in the second set of two or more loci, and
when the contribution of the sixth fluorescent moiety to the fluorescent signal detected in the reaction vessel satisfies the threshold contribution, performing a third plurality of secondary allele detection assays, wherein each secondary allele detection assay in the third plurality of secondary allele detection assays determines the allele status at one respective genomic locus in the third set of two or more genomic loci, thereby determining the allele status at each respective loci in the third set of two or more genomic loci.

45. The method of claim 44, wherein the first, second, and third set of two or more genomic loci comprise:
the human alleles corresponding to the SNPs rs5030863, rs28371685, and rs5030867,
the human alleles corresponding to the SNPs rs17884712, rs72552267, and rs72558187, and
the human alleles corresponding to the SNPs rs5030862 and rs56337013, respectively.

46. The method of any one of claims 38-45, wherein:
the plurality of genomic loci comprises the human allele corresponding to the rs7997012 SNP, and
when the subject is determined to carry the rs7997012 SNP, the one or more recommendations include assigning antidepressant therapy comprising administration of a selective serotonin reuptake inhibitor (SSRI).

47. The method of claim 46, wherein the SSRI is citalopram.

48. The method of any one of claims 38-47, wherein:
the plurality of genomic loci comprises the human allele corresponding to the rs3813929 SNP, and
when the subject is determined to carry the rs3813929 SNP, the one or more recommendations include assigning antidepressant therapy comprising administering a low dose of an antipsychotics.

49. The method of claim 48, wherein the antipsychotics is amisulpride, clozapine, haloperidol, iloperidone, olanzapine, quetiapine, risperidone, or ziprasidone.

50. The method of any one of claims 38-49, wherein:
the plurality of genomic loci comprises the human allele corresponding to the rs1045642 SNP, and
when the subject is determined to carry the rs1045642 SNP, the one or more recommendations include assigning antidepressant therapy comprising administration of a low dose of an antipsychotics.

51. The method of claim 50, wherein the antipsychotics is chlorpromazine.

52. The method of any one of claims 38-51, wherein:
the plurality of genomic loci comprises the human allele corresponding to the rs2032583 SNP, and
when the subject is determined to carry the rs2032583 SNP, the one or more recommendations include assigning antidepressant therapy comprising administration of a SSRI or a tricyclic antidepressant (TCA).

53. The method of claim 52, wherein the SSRI is citalopram, fluvoxamine, paroxetine, or sertraline.

54. The method of claim 52, wherein the TCA is amitriptyline.

55. The method of any one of claims 38-54, wherein:
the plurality of genomic loci comprises the human allele corresponding to the rs1800544 SNP, and
when the subject is determined to carry the rs1800544 SNP, the one or more recommendations include assigning antidepressant therapy comprising administration of a serotonin-norepinephrine reuptake inhibitor (SNRI).

56. The method of claim 55, wherein the SNRI is milnacipran.

57. The method of any one of claims 38-56, wherein:
the plurality of genomic loci comprises the human allele corresponding to the rs10994336 SNP, and
when the subject is determined to carry the rs10994336 SNP, the one or more recommendations include administering sodium channel modulating agents.

58. The method of claim 57, wherein the sodium channel modulating agent is lamotrigine.

59. The method of any one of claims 38-57, wherein:
the plurality of genomic loci comprises the human allele corresponding to the rs6265 SNP, and
when the subject is determined to carry the rs6265 SNP, the one or more recommendations include assigning antidepressant therapy comprising administration of a SSRI or an antipsychotics.

60. The method of claim 59, wherein the SSRI is paroxetine.

61. The method of claim 59, wherein the antipsychotics is clozapine.

62. The method of any one of claims 38-61, wherein:
the plurality of genomic loci comprises the human allele corresponding to the rs1006737 SNP, and
when the subject is determined to carry the rs1006737 SNP, the one or more recommendations include assigning antidepressant therapy comprising administering a low dose of SSRI.

63. The method of claim 62, wherein the SSRI is citalopram.

64. The method of any one of claims 38-63, wherein:
the plurality of genomic loci comprises the human allele corresponding to the rs4680 SNP, and
when the subject is determined to carry the rs4680 SNP, the one or more recommendations include assigning antidepressant therapy comprising administration of a SSRI or a SNRI.

65. The method of claim 64, wherein the SSRI is paroxetine.

66. The method of claim 64, wherein the SNRI is venlafaxine.

67. The method of any one of claims 38-66, wherein:
the plurality of genomic loci comprises the human allele corresponding to the rs2470890 SNP, and
when the subject is determined to carry the rs2470890 SNP, the one or more recommendations include assigning antidepressant therapy comprising administering a low dose of antipsychotics.

68. The method of claim 67, wherein the antipsychotics is clozapine.

69. The method of any one of claims 38-67, wherein:
the plurality of genomic loci comprises the human allele corresponding to the rs2069514 SNP, and
when the subject is determined to carry the rs2069514 SNP, the one or more recommendations include assigning antidepressant therapy comprising administration of a low dose of antipsychotics.

70. The method of any one of claims 38-69, wherein:
the plurality of genomic loci comprises the human allele corresponding to the rs35694136 SNP, and
when the subject is determined to carry the rs35694136 SNP, the one or more recommendations include assigning antidepressant therapy comprising administration of a low dose of antipsychotics.

71. The method of any one of claims 38-70, wherein:
the plurality of genomic loci comprises the human allele corresponding to the rs2069526 SNP, and
when the subject is determined to carry the rs2069526 SNP, the one or more recommendations include assigning antidepressant therapy comprising administering a low dose of a SSRI.

72. The method of claim 71, wherein the SSRI is escitalopram.

73. The method of any one of claims 38-72, wherein:
the plurality of genomic loci comprises the human allele corresponding to the rs762551 SNP, and
when the subject is determined to carry the rs762551 SNP, the one or more recommendations include assigning antidepressant therapy comprising administration of a high dose of a SSRI.

74. The method of claim 73, wherein the SSRI is paroxetine.

75. The method of any one of claims 38-74, wherein:
the plurality of genomic loci comprises the human allele corresponding to the rs72547513 SNP, and
when the subject is determined to carry the rs72547513 SNP, the one or more recommendations include administering a low dose of pharmaceutical agents that are normally metabolized by cytochrome P450 1A2.

76. The method of any one of claims 38-75, wherein:
the plurality of genomic loci comprises the human allele corresponding to the rs2279343 SNP, and
when the subject is determined to carry the rs2279343 SNP, the one or more recommendations include assigning therapy for substance abuse.

77. The method of claim 76, wherein the substance is heroin, and
the therapy comprises administering a high dose of methadone.

78. The method of claim 76, wherein the substance is nicotine, and
the therapy comprises administering bupropion.

79. The method of any one of claims 38-78, wherein:
the plurality of genomic loci comprises the human allele corresponding to the rs3211371 SNP, and
when the subject is determined to carry the rs3211371 SNP, the one or more recommendations include assigning non-heroin therapy comprising administration of a high dose of methadone.

80. The method of any one of claims 38-79, wherein:
the plurality of genomic loci comprises the human allele corresponding to the rs3745274 SNP, and
when the subject is determined to carry the rs3745274 SNP, the one or more recommendations include assigning therapy for substance abuse.

81. The method of claim 80, wherein the substance is heroin, and
the therapy comprises administering a high dose of methadone.

82. The method of claim 80, wherein the substance is nicotine, and
the therapy comprises administering bupropion.

83. The method of any one of claims 38-82, wherein:
the plurality of genomic loci comprises the human allele corresponding to the rs2279343 SNP, and
when the subject is determined to carry the rs2279343 SNP, the one or more recommendations include assigning therapy for substance abuse.

84. The method of claim 83, wherein the substance is heroin, and
the therapy comprises administering a high dose of methadone.

85. The method of claim 83, wherein the substance is nicotine, and
the therapy comprises administering bupropion.

86. The method of any one of claims 38-85, wherein:
the plurality of genomic loci comprises the human allele corresponding to the rs2279343 SNP, and
when the subject is determined to carry the rs2279343 SNP, the one or more recommendations include assigning antidepressant therapy comprising administration of mirtazapine.

87. The method of any one of claims 38-86, wherein:
the plurality of genomic loci comprises the human allele corresponding to the rs4244285 SNP, and
when the subject is determined to carry the rs4244285 SNP, the one or more recommendations include assigning antidepressant therapy comprising administration of a low dose of a TCA or a SSRI.

88. The method of claim 87, wherein the TCA is amitriptyline.

89. The method of claim 87, wherein the SSRI is citalopram or escitalopram.

90. The method of any one of claims 38-89, wherein:
the plurality of genomic loci comprises the human allele corresponding to the rs17878459 SNP, and
when the subject is determined to carry the rs17878459 SNP, the one or more recommendations include assigning antidepressant therapy comprising administering a low dose of antipsychotics.

91. The method of any one of claims 38-90, wherein:
the plurality of genomic loci comprises the human allele corresponding to the rs4986893 SNP, and
when the subject is determined to carry the rs4986893 SNP, the one or more recommendations include assigning antidepressant therapy comprising administering a low dose of a SSRI.

92. The method of claim 91, wherein the SSRI is citalopram or escitalopram.

93. The method of any one of claims 38-92, wherein:
the plurality of genomic loci comprises the human allele corresponding to the rs57081121 SNP, and
when the subject is determined to carry the rs57081121 SNP, the one or more recommendations include assigning antidepressant therapy comprising administering a low dose of a SSRI.

94. The method of claim 93, wherein the SSRI is citalopram or escitalopram.

95. The method of any one of claims 38-94, wherein:
the plurality of genomic loci comprises the human allele corresponding to the rs28399504 SNP, and
when the subject is determined to carry the rs28399504 SNP, the one or more recommendations include assigning antidepressant therapy comprising administration of a low dose of a SSRI.

96. The method of claim 95, wherein the SSRI is citalopram or escitalopram.

97. The method of any one of claims 38-96, wherein:
the plurality of genomic loci comprises the human allele corresponding to the rs56337013 SNP, and
when the subject is determined to carry the rs56337013 SNP, the one or more recommendations include administering a low dose of a pharmaceutical agent that is metabolized by cytochrome P450 2C19 (CYP2C19) when the subject is determined to carry the rs56337013 SNP.

98. The method of any one of claims 38-97, wherein:
the plurality of genomic loci comprises the human allele corresponding to the rs72552267 SNP, and
when the subject is determined to carry the rs72552267 SNP, the one or more recommendations include administering a low dose of a pharmaceutical agent that is metabolized by cytochrome P450 2C19 (CYP2C19) when the subject is determined to carry the rs72552267 SNP.

99. The method of any one of claims 38-98, wherein:
the plurality of genomic loci comprises the human allele corresponding to the rs72558186 SNP, and
when the subject is determined to carry the rs72558186 SNP, the one or more recommendations include administering a low dose of a pharmaceutical agent that is metabolized by cytochrome P450 2C19 (CYP2C19).

100. The method of any one of claims 38-99, wherein:
the plurality of genomic loci comprises the human allele corresponding to the rs41291556 SNP, and
when the subject is determined to carry the rs41291556 SNP, the one or more recommendations include administering a low dose of a pharmaceutical agent that is metabolized by cytochrome P450 2C19 (CYP2C19).

101. The method of any one of claims 38-100, wherein:
the plurality of genomic loci comprises the human allele corresponding to the rs17884712 SNP, and
when the subject is determined to carry the rs17884712 SNP, the one or more recommendations include administering a low dose of a pharmaceutical agent that is metabolized by cytochrome P450 2C19 (CYP2C19) when the subject is determined to carry the rs17884712 SNP.

102. The method of any one of claims 38-101, wherein:
the plurality of genomic loci comprises the human allele corresponding to the rs6413438 SNP, and
when the subject is determined to carry the rs6413438 SNP, the one or more recommendations include administering a low dose of a pharmaceutical agent that is metabolized by cytochrome P450 2C19 (CYP2C19).

103. The method of any one of claims 38-102, wherein:
the plurality of genomic loci comprises the human allele corresponding to the rs12248560 SNP, and
when the subject is determined to carry the rs12248560 SNP, the one or more recommendations include assigning antidepressant therapy comprising administration of a low dose of a SSRI.

104. The method of claim 103, wherein the SSRI is citalopram or escitalopram.

105. The method of any one of claims 38-104, wherein:
the plurality of genomic loci comprises the human allele corresponding to the rs12248560 SNP, and
when the subject is determined to carry the rs12248560 SNP, the one or more recommendations include assigning antidepressant therapy comprising administration of a low dose of a TCA.

106. The method of claim 105, wherein the TCA is amitriptyline or clomipramine.

107. The method of any one of claims 38-106, wherein:
the plurality of genomic loci comprises the human allele corresponding to the rs12769205 SNP, and
when the subject is determined to carry the rs12769205 SNP, the one or more recommendations include assigning antidepressant therapy comprising administration of a low dose of a SSRI or a TCA.

108. The method of claim 107, wherein the SSRI is sertraline or escitalopram.

109. The method of claim 107, wherein the TCA is amitriptyline or imipramine.

110. The method of any one of claims 38-109, wherein:
the plurality of genomic loci comprises the human allele corresponding to the rs3758581 SNP, and
when the subject is determined to carry the rs3758581 SNP, the one or more recommendations include assigning antidepressant therapy comprising administration of a low dose of a SSRI or a TCA.

111. The method of claim 110, wherein the SSRI is sertraline or escitalopram.

112. The method of claim 110, wherein the TCA is amitriptyline or imipramine.

113. The method of any one of claims 38-112, wherein:
the plurality of genomic loci comprises the human allele corresponding to the rs1799853 SNP, and
when the subject is determined to carry the rs1799853 SNP, the one or more recommendations include assigning psychotropic therapy comprising administration of a low dose of valproic acid.

114. The method of any one of claims 38-113, wherein:
the plurality of genomic loci comprises the human allele corresponding to the rs1057910 SNP, and
when the subject is determined to carry the rs1057910 SNP, the one or more recommendations include assigning psychotropic therapy comprising administration of a low dose of a TCA or valproic acid.

115. The method of claim 114, wherein the TCA is trimipramine or doxepin.

116. The method of any one of claims 38-115, wherein:
the plurality of genomic loci comprises the human allele corresponding to the rs56165452 SNP, and

117. when the subject is determined to carry the rs56165452 SNP, the one or more recommendations include administering low dose of a pharmaceutical agent that is metabolized by cytochrome P450 2C9. The method of any one of claims 38-116, wherein:
the plurality of genomic loci comprises the human allele corresponding to the rs28371686 SNP, and
when the subject is determined to carry the rs28371686 SNP, the one or more recommendations include administering low dose of a pharmaceutical agent that is metabolized by cytochrome P450 2C9.

118. The method of any one of claims 38-117, wherein:
the plurality of genomic loci comprises the human allele corresponding to the rs9332131 SNP, and
when the subject is determined to carry the rs9332131 SNP, the one or more recommendations include administering a low dose of an antiepileptic drug (AED).

119. The method of claim 118, wherein the AED is phenytoin.

120. The method of any one of claims 38-119, wherein:
the plurality of genomic loci comprises the human allele corresponding to the rs7900194 SNP, and
when the subject is determined to carry the rs7900194 SNP, the one or more recommendations include administering a low dose of an antiepileptic drug (AED).

121. The method of claim 120, wherein the AED is phenytoin.

122. The method of any one of claims 38-121, wherein:
the plurality of genomic loci comprises the human allele corresponding to the rs28371685 SNP, and
when the subject is determined to carry the rs28371685 SNP, the one or more recommendations include administering a low dose of a pharmaceutical agent that is metabolized by cytochrome P450 2C9 (CYP2C9) when the subject is determined to carry the rs28371685 SNP.

123. The method of any one of claims 38-122, wherein:
the plurality of genomic loci comprises the human allele corresponding to the rs72558187 SNP, and
when the subject is determined to carry the rs72558187 SNP, the one or more recommendations include administering a low dose of an antiepileptic drug (AED).

124. The method of claim 123, wherein the AED is phenytoin.

125. The method of any one of claims 38-124, wherein:
the plurality of genomic loci comprises the human allele corresponding to the rs1135840 SNP, and
when the subject is determined to carry the rs1135840 SNP, the one or more recommendations include assigning antidepressant therapy comprising administration of a TCA, a SSRI, or a norepinephrine reuptake inhibitor (NRI).

126. The method of claim 125, wherein the TCA is imipramine, amitriptyline, trimipramine, clomipramine, desipramine, or doxepin.

127. The method of claim 125, wherein the SSRI is paroxetine, citalopram, or escitalopram.

128. The method of claim 127, wherein the NRI is atomoxetine.

129. The method of any one of claims 38-128, wherein:
the plurality of genomic loci comprises the human allele corresponding to the rs16947 SNP, and
when the subject is determined to carry the rs16947 SNP, the one or more recommendations include assigning antidepressant therapy comprising administration of a TCA, a SSRI, or a norepinephrine reuptake inhibitor (NRI).

130. The method of claim 129, wherein the TCA is imipramine, amitriptyline, trimipramine, clomipramine, desipramine, or doxepin.

131. The method of claim 129, wherein the SSRI is paroxetine, citalopram, or escitalopram.

132. The method of claim 129, wherein the NRI is atomoxetine.

133. The method of any one of claims 38-132, wherein:
the plurality of genomic loci comprises the human allele corresponding to the rs1135824 SNP, and
when the subject is determined to carry the rs1135824 SNP, the one or more recommendations include assigning antidepressant therapy comprising administration of a low dose of a SSRI, a TCA or a NRI.

134. The method of claim 133, wherein the SSRI is paroxetine, citalopram, fluvoxamine, fluoxetine, or escitalopram.

135. The method of claim 133, wherein the TCA is imipramine, amitriptyline, trimipramine, clomipramine, desipramine, doxepin, trimipramine, or nortriptyline.

136. The method of claim 133, wherein the NRI is atomoxetine.

137. The method of any one of claims 38-136, wherein:
the plurality of genomic loci comprises the human allele corresponding to the rs35742686 SNP, and
when the subject is determined to carry the rs35742686 SNP, the one or more recommendations include assigning antidepressant therapy comprising administration of a low dose of a SSRI, a TCA or a NRI.

138. The method of claim 137, wherein the SSRI is paroxetine, citalopram, fluvoxamine, fluoxetine, or escitalopram.

139. The method of claim 137, wherein the TCA is imipramine, amitriptyline, trimipramine, clomipramine, desipramine, doxepin, trimipramine, or nortriptyline.

140. The method of claim 137, wherein the NRI is atomoxetine.

141. The method of any one of claims 38-140, wherein:
the plurality of genomic loci comprises the human allele corresponding to the rs3892097 SNP, and
when the subject is determined to carry the rs3892097 SNP, the one or more recommendations include assigning antidepressant therapy comprising administration of a low dose of a SSRI, a TCA, or a NRI.

142. The method of claim 141, wherein the SSRI is paroxetine, citalopram, fluvoxamine, fluoxetine, or escitalopram.

143. The method of claim 141, wherein the TCA is imipramine, amitriptyline, trimipramine, clomipramine, desipramine, doxepin, trimipramine, or nortriptyline.

144. The method of claim 141, wherein the NRI is atomoxetine.

145. The method of any one of claims 38-144, wherein:
the plurality of genomic loci comprises the human allele corresponding to the rs5030655 SNP, and
when the subject is determined to carry the rs5030655 SNP, the one or more recommendations include assigning antidepressant therapy comprising administration of a low dose of a SSRI, a TCA, or a NRI.

146. The method of claim 145, wherein the SSRI is paroxetine, citalopram, fluvoxamine, fluoxetine, or escitalopram.

147. The method of claim 145, wherein the TCA is imipramine, amitriptyline, trimipramine, clomipramine, desipramine, doxepin, trimipramine, or nortriptyline.

148. The method of claim 145, wherein the NRI is atomoxetine.

149. The method of any one of claims 38-148, wherein:
the plurality of genomic loci comprises the human allele corresponding to the rs5030867 SNP, and
when the subject is determined to carry the rs5030867 SNP, the one or more recommendations include administering a low dose of an antipsychotics when the subject is determined to carry the rs5030867 SNP.

150. The method of any one of claims 38-149, wherein:
the plurality of genomic loci comprises the human allele corresponding to the rs5030865 SNP, and
when the subject is determined to carry the rs5030865 SNP, the one or more recommendations include assigning antidepressant therapy comprising administering a low dose of an antipsychotics.

151. The method of claim 150, wherein the antipsychotics is risperidone.

152. The method of any one of claims 38-151, wherein:
the plurality of genomic loci comprises the human allele corresponding to the rs5030656 SNP, and
when the subject is determined to carry the rs5030656 SNP, the one or more recommendations include administering a low dose of a pharmaceutical agent that is metabolized by cytochrome P450 2D6.

153. The method of any one of claims 38-152, wherein:
the plurality of genomic loci comprises the human allele corresponding to the rs1065852 SNP, and
when the subject is determined to carry the rs1065852 SNP, the one or more recommendations include assigning antidepressant therapy comprising administration of a low dose of a SSRI, a TCA, a NRI or an antipsychotics.

154. The method of claim 153, wherein the SSRI is paroxetine, citalopram, fluvoxamine, fluoxetine, or escitalopram.

155. The method of claim 154, wherein the TCA is imipramine, amitriptyline, trimipramine, clomipramine, desipramine, doxepin, or nortriptyline.

156. The method of claim 155, wherein the NRI is atomoxetine.

157. The method of any one of claims 38-156, wherein:
the plurality of genomic loci comprises the human allele corresponding to the rs5030863 SNP, and
when the subject is determined to carry the rs5030863 SNP, the one or more recommendations include administering a low dose of a pharmaceutical agent that is metabolized by cytochrome P450 2D6 (CYP2D6) when the subject is determined to carry the rs5030863 SNP.

158. The method of any one of claims 38-157, wherein:
the plurality of genomic loci comprises the human allele corresponding to the rs5030862 SNP, and
when the subject is determined to carry the rs5030862 SNP, the one or more recommendations include administering a low dose of a pharmaceutical agent that is metabolized by cytochrome P450 2D6 (CYP2D6) when the subject is determined to carry the rs5030862 SNP.

159. The method of any one of claims 38-158, wherein:
the plurality of genomic loci comprises the human allele corresponding to the rs5030865(T) SNP, and
when the subject is determined to carry the rs5030865(T) SNP, the one or more recommendations include assigning antidepressant therapy comprising administration of a low dose of an antipsychotics.

160. The method of claim 159, wherein the antipsychotics is risperidone.

161. The method of any one of claims 38-160, wherein:
the plurality of genomic loci comprises the human allele corresponding to the rs774671100 SNP, and
when the subject is determined to carry the rs774671100 SNP, the one or more recommendations include administering a low dose of pharmaceutical agents that are normally metabolized by cytochrome P450 2D6.

162. The method of any one of claims 38-161, wherein:
the plurality of genomic loci comprises the human allele corresponding to the rs28371706 SNP, and
when the subject is determined to carry the rs28371706 SNP, the one or more recommendations include assigning antidepressant therapy comprising administration of a TCA.

163. The method of claim 161, wherein the TCA is haloperidol.

164. The method of claim 161, wherein the TCA is desipramine or nortriptyline, and a low dose of the TCA is administered.

165. The method of any one of claims 38-164, wherein:
the plurality of genomic loci comprises the human allele corresponding to the rs16947 SNP, and
when the subject is determined to carry the rs16947 SNP, the one or more recommendations include assigning antidepressant therapy comprising administration of a TCA.

166. The method of claim 165, wherein the TCA is haloperidol.

167. The method of claim 165, wherein the TCA is desipramine or nortriptyline, and a low dose of the TCA is administered.

168. The method of any one of claims 38-167, wherein:
the plurality of genomic loci comprises the human allele corresponding to the rs61736512 SNP, and
when the subject is determined to carry the rs61736512 SNP, the one or more recommendations include administering a low dose of a pharmaceutical agent that is metabolized by cytochrome P450 2D6 (CYP2D6).

169. The method of any one of claims 38-168, wherein:
the plurality of genomic loci comprises the human allele corresponding to the rs1058164 SNP, and
when the subject is determined to carry the rs1058164 SNP, the one or more recommendations include administering a low dose of a pharmaceutical agent that is metabolized by cytochrome P450 2D6 (CYP2D6).

170. The method of any one of claims 38-169, wherein:
the plurality of genomic loci comprises the human allele corresponding to the rs59421388 SNP, and
when the subject is determined to carry the rs59421388 SNP, the one or more recommendations include administering a low dose of a pharmaceutical agent that is metabolized by cytochrome P450 2D6 (CYP2D6).

171. The method of any one of claims 38-170, wherein:
the plurality of genomic loci comprises the human allele corresponding to the rs28371725 SNP, and
when the subject is determined to carry the rs28371725 SNP, the one or more recommendations include assigning antidepressant therapy comprising administration of a TCA, a SSRI or a SNRI.

172. The method of claim 171, wherein the SSRI is citalopram or escitalopram.

173. The method of claim 171, wherein the TCA is desipramine, aripiprazole, haloperidol, levomepromazine, quetiapine, or risperidone, and a low dose of the TCA is administered.

174. The method of claim 171, wherein the SSRI is desipramine, aripiprazole, haloperidol, levomepromazine, or quetiapine, and a low dose of the SSRI is administered.

175. The method of any one of claims 38-174, wherein:
the plurality of genomic loci comprises the human allele corresponding to the rs35599367 SNP, and
when the subject is determined to carry the rs35599367 SNP, the one or more recommendations include assigning antidepressant therapy comprising administration of a low dose of an antipsychotics.

176. The method of claim 175, wherein the antipsychotics is risperidone.

177. The method of any one of claims 38-176, wherein:
the plurality of genomic loci comprises the human allele corresponding to the rs776746 SNP, and
when the subject is determined to carry the rs776746 SNP, the one or more recommendations include administering a low dose of pharmaceutical agents that are normally metabolized by cytochrome P450 3A5.

178. The method of any one of claims 38-177, wherein:
the plurality of genomic loci comprises the human allele corresponding to the rs10264272 SNP, and
when the subject is determined to carry the rs10264272 SNP, the one or more recommendations include administering a low dose of pharmaceutical agents that are normally metabolized by cytochrome P450 3A5.

179. The method of any one of claims 38-178, wherein:
the plurality of genomic loci comprises the human allele corresponding to the rs41303343 SNP, and
when the subject is determined to carry the rs41303343 SNP, the one or more recommendations include administering a low dose of pharmaceutical agents that are normally metabolized by cytochrome P450 3A5.

180. The method of any one of claims 38-179, wherein:
the plurality of genomic loci comprises the human allele corresponding to the rs1799732 SNP, and
when the subject is determined to carry the rs1799732 SNP, the one or more recommendations include assigning antidepressant therapy comprising administration of a antipsychotics.

181. The method of claim 180, wherein the antipsychotics is aripiprazole, bromperidol, chlorpromazine, clozapine, nemonapride, olanzapine, or risperidone.

182. The method of claim 181, wherein the antipsychotics is risperidone, and a low dose of the antipsychotics is administered.

183. The method of any one of claims 38-182, wherein:
the plurality of genomic loci comprises the human allele corresponding to the rs1799732 SNP, and
when the subject is determined to carry the rs1799732 SNP, the one or more recommendations include assigning therapy for tobacco use disorder comprising administration of a non-nicotine replacement.

184. The method of any one of claims 38-183, wherein:
the plurality of genomic loci comprises the human allele corresponding to the rs2832407 SNP, and
when the subject is determined to carry the rs2832407 SNP, the one or more recommendations include assigning therapy for alcohol abuse comprising administration of a low dose of topiramate.

185. The method of any one of claims 38-184, wherein:
the plurality of genomic loci comprises the human allele corresponding to the rs1061235 SNP, and
when the subject is determined to carry the rs1061235 SNP, the one or more recommendations include administering a lose dose of an anticonvulsant or an AED.

186. The method of claim 185, wherein the anticonvulsant is carbamazepine, oxcarbazepine, or lamotrigine.

187. The method of claim 185, wherein the AED is phenytoin.

188. The method of any one of claims 38-187, wherein:
the plurality of genomic loci comprises the human allele corresponding to the rs2395148 SNP, and
when the subject is determined to carry the rs2395148 SNP, the one or more recommendations include administering a lose dose of an anticonvulsant or an AED.

189. The method of claim 188, wherein the anticonvulsant is carbamazepine, oxcarbazepine, or lamotrigine.

190. The method of claim 188, wherein the AED is phenytoin.

191. The method of any one of claims 38-190, wherein:
the plurality of genomic loci comprises the human allele corresponding to the rs489693 SNP, and
when the subject is determined to carry the rs489693 SNP, the one or more recommendations include assigning antidepressant therapy comprising administration of a low dose of an antipsychotics.

192. The method of claim 191, wherein the antipsychotics is amisulpride, aripiprazole, clozapine, haloperidol, olanzapine, paliperidone, quetiapine, risperidone, or ziprasidone.

193. The method of any one of claims 38-192, wherein:
the plurality of genomic loci comprises the human allele corresponding to the rs1801131 SNP, and
when the subject is determined to carry the rs1801131 SNP, the one or more recommendations include assigning antidepressant therapy comprising administration of a low dose of antipsychotics.

194. The method of claim 193, wherein the antipsychotics is olanzapine or clozapine.

195. The method of any one of claims 38-194, wherein:
the plurality of genomic loci comprises the human allele corresponding to the rs1801131 SNP, and
when the subject is determined to carry the rs1801131 SNP, the one or more recommendations include assigning anti-depression therapy comprising administration of 1-methylfolate or vitamin B-complex.

196. The method of any one of claims 38-195, wherein:
the plurality of genomic loci comprises the human allele corresponding to the rs1801133 SNP, and
when the subject is determined to carry the rs1801133 SNP, the one or more recommendations include assigning antidepressant therapy comprising administration of a low dose of antipsychotics.

197. The method of any one of claims 38-196, wherein:
the plurality of genomic loci comprises the human allele corresponding to the rs1801133 SNP, and
when the subject is determined to carry the rs1801133 SNP, the one or more recommendations include assigning anti-depression therapy comprising administration of 1-methylfolate or vitamin B-complex.

198. The method of any one of claims 38-197, wherein:
the plurality of genomic loci comprises the human allele corresponding to the rs1801133 SNP, and
when the subject is determined to carry the rs1801133 SNP, the one or more recommendations include assigning therapy for cocaine abuse comprising administration of disulfiram.

199. The method of any one of claims 38-198, wherein:
the plurality of genomic loci comprises the human allele corresponding to the rs1799971 SNP, and
when the subject is determined to carry the rs1799971 SNP, the one or more recommendations include assigning therapy for substance abuse.

200. The method of claim 199, wherein the substance is tobacco, and
the therapy comprises administering a nicotine-replacement.

201. The method of claim 199, wherein the substance is opioid, and
the therapy comprises administering a low dose of methadone.

202. The method of any one of claims 38-201, wherein:
the plurality of genomic loci comprises the human allele corresponding to the rs25531 SNP, and
when the subject is determined to carry the rs25531 SNP, the one or more recommendations include assigning antidepressant therapy comprising administration of a low dose of SSRI.

203. The method of claim 202, wherein the SSRI is fluoxetine or citalopram.

204. The method of any one of claims 38-203, wherein:
the plurality of genomic loci comprises the human allele corresponding to the rs63749047 SNP, and
when the subject is determined to carry the rs63749047 SNP, the one or more recommendations include assigning antidepressant therapy comprising administration of a low dose of SSRI.

205. The method of claim 204, wherein the SSRI is citalopram, escitalopram, fluoxetine, fluvoxamine, paroxetine, or sertraline.

206. The method of any one of claims 38-205, wherein:
the plurality of genomic loci comprises the human allele corresponding to the rs2011425 SNP, and
when the subject is determined to carry the rs2011425 SNP, the one or more recommendations include administering a low dose of lamotrigine, asenapine, or trifluoperazine.

207. The method of any one of claims 38-206, wherein:
the plurality of genomic loci comprises the human allele corresponding to the rs1902023 SNP, and
when the subject is determined to carry the rs1902023 SNP, the one or more recommendations include assigning psychotropic therapy comprising administration of a low dose of a benzodiazepine (BZD).

208. The method of claim 207, wherein the BZD is clonazepam, diazepam, lorazepam, oxazepam, or temazepam.

209. The method of claim 38 wherein the neuropsychiatric disorder is major depression, anxiety disorder, obsessive-compulsive disorder, attention deficit hyperactivity disorder (ADHD), bipolar disorder, post-traumatic stress disorder (PTSD), autism, schizophrenia, personality disorder, chronic pain, or substance abuse.

210. A method for providing treatment guidance in a subject, the method comprising:
a) determining the allele status for a plurality of genomic loci, wherein the determining comprises:
i) amplifying, in a first single in vitro reaction vessel, a first set of two or more genomic loci in the plurality of genomic loci, by polymerase chain reaction (PCR), from nucleic acids isolated from a sample obtained from the subject, in the presence of a plurality of fluorescently-labeled detection reagents, wherein the plurality of fluorescently-labeled detection reagents includes, for each respective genomic locus in the two or more genomic loci:
a first detection reagent that is specific for the presence of a first allele at the respective genomic locus, wherein the first allele is the most prevalent allele in a population of the species of the subject and the first detection reagent is labeled with a first fluorescent moiety, and
a second detection reagent that is specific for the presence of a second allele at the respective genomic locus, wherein the second allele is a minor allele in the population and the second detection reagent is labeled with a second fluorescent moiety that is distinguishable from the first fluorescent moiety;
ii) during or after the amplifying i), detecting a fluorescent signal corresponding to the first fluorescent moiety and the second fluorescent moiety in the first single reaction vessel; and
iii) responsive to the detecting ii):
when the contribution of the second fluorescent moiety to the fluorescent signal detected in the reaction vessel does not satisfy a threshold contribution, determining that the subject is homozygous for the first allele at each respective loci in the first set of two or more genomic loci, and
when the contribution of the second fluorescent moiety to the fluorescent signal detected in the reaction vessel satisfies the threshold contribution, performing a first plurality of secondary allele detection assays, wherein each secondary allele detection assay in the first plurality of secondary allele detection assays determines the allele status at one respective genomic locus in the first set of two or more genomic loci, thereby determining the allele status at each respective loci in the first set of two or more genomic loci;
b) associating the allele status determined for the plurality of genomic loci with one or more recommendations for the treatment of the neuropsychiatric disorder; and
c) generating a patient-specific report comprising the one or more recommendations for the treatment of a condition in fulfillment of an ICD-10 code selected from the group consisting of F31.0, F31.1, F31.2, F31.3, F31.5, F31.6, F31.7, F31.8, F31.9, F32.0, F32.2, F32.3, F32.4, F32.5, F32.8, F32.9, F33.0, F33.1, F33.2, F33.3, F33.4, F33.8, F33.9, F40.0, F40.1, F40.2, F40.8, F40.9, F41.0, F41.1, F41.3, F41.8, F41.9, F42.2, F42.3, F42.4, F42.8, F42.9, F60.5, F90.0, F90.1, F90.2, F90.8, F90.9, F43.1, F84.0, F20.0, F20.1, F20.2, F20.3, F20.5, F20.8, F20.9, F60.0, F60.1, F60.2, F60.3, F60.4, F60.5, F60.6, F60.7, F60.8, F60.9, F07.0, F07.8, F07.9, G89.2, G89.4, F10.1, F10.2, F10.9, F11.1, F11.2, F11.9, F12.1, F12.2, F12.9, F13.1, F13.2, F13.9, F14.1, F14.2, F14.9, F15.1, F15.2, F15.9, F16.1, F16.2, F16.9, F17.2, F18.1, F18.2, F18.9, F19.1, F19.2, F19.9, F55.0, F55.1, F55.2, F55.3, F55.4, and F55.8.

211. The method of claim 210, wherein the plurality of genomic loci comprises one or more genomic loci corresponding to a SNP selected from the group consisting of rs7997012, rs3813929, rs1045642, rs2032583, rs1800544, rs10994336, rs6265, rs1006737, rs4680, rs2470890 (CYP1A2*1B), rs2069514, rs35694136, rs2069526 (CYP1A2*1E), rs762551, rs12720461 (CYP1A2*1K), rs2069526 (CYP1A2*1K), rs72547513, rs2279343 (CYP2B6*4), rs3211371, rs3745274 (CYP2B6*6), rs2279343 (CYP2B6*6), rs4244285, rs17878459 (CYP2C19*2B), rs4986893 (CYP2C19*3), rs57081121 (CYP2C19*3), rs28399504, rs56337013, rs72552267, rs72558186, rs41291556, rs17884712, rs6413438, rs12248560, rs12769205 (CYP2C19*35), rs3758581 (CYP2C19*35), rs1799853, rs1057910, rs56165452, rs28371686, rs9332131, rs7900194, rs28371685, rs72558187, rs7900194 (CYP2C9*27), rs16947 (CYP2D6*2), rs1135840 (CYP2D6*2), rs1135824 (CYP2D6*3), rs35742686 (CYP2D6*3), rs3892097, rs5030655, rs5030867, rs5030865, rs5030656, rs1065852, rs5030863, rs5030862, rs5030865, rs774671100, rs28371706 (CYP2D6*17), rs16947 (CYP2D6*17), rs61736512 (CYP2D6*29), rs1058164 (CYP2D6*29), rs16947 (CYP2D6*29), rs59421388 (CYP2D6*29), rs1135840 (CYP2D6*29), rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023.

212. The method of claim 210, wherein the plurality of genomic loci comprises at least the genomic loci corresponding to SNPs rs7997012, rs3813929, rs1045642, rs2032583, rs1800544, rs10994336, rs6265, rs1006737, rs4680, rs2470890 (CYP1A2*1B), rs2069514, rs35694136, rs2069526 (CYP1A2*1E), rs762551, rs12720461 (CYP1A2*1K), rs2069526 (CYP1A2*1K), rs72547513, rs2279343 (CYP2B6*4), rs3211371, rs3745274 (CYP2B6*6), rs2279343 (CYP2B6*6), rs4244285, rs17878459 (CYP2C19*2B), rs4986893 (CYP2C19*3), rs57081121 (CYP2C19*3), rs28399504, rs56337013, rs72552267, rs72558186, rs41291556, rs17884712, rs6413438, rs12248560, rs12769205 (CYP2C19*35), rs3758581 (CYP2C19*35), rs1799853, rs1057910, rs56165452, rs28371686, rs9332131, rs7900194, rs28371685, rs72558187, rs7900194 (CYP2C9*27), rs16947 (CYP2D6*2), rs1135840 (CYP2D6*2), rs1135824 (CYP2D6*3), rs35742686 (CYP2D6*3), rs3892097, rs5030655, rs5030867, rs5030865, rs5030656, rs1065852, rs5030863, rs5030862, rs5030865, rs774671100, rs28371706 (CYP2D6*17), rs16947 (CYP2D6*17), rs61736512 (CYP2D6*29), rs1058164 (CYP2D6*29), rs16947 (CYP2D6*29), rs59421388 (CYP2D6*29), rs1135840 (CYP2D6*29), rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023.

213. The method of any one of claims 210-212, wherein the first set of two or more genomic loci comprises one of:
the human alleles corresponding to the SNPs rs5030863, rs28371685, and rs5030867,
the human alleles corresponding to the SNPs rs17884712, rs72552267, and rs72558187, or
the human alleles corresponding to the SNPs rs5030862 and rs56337013.

214. The method of any one of claims 210-213, wherein the determining a) further comprises:
iv) amplifying, in a second single in vitro reaction vessel, a second set of two or more genomic loci in the plurality of genomic loci, by polymerase chain reaction (PCR), from nucleic acids isolated from a sample obtained from the subject, in the presence of a plurality of fluorescently-labeled detection reagents, wherein the plurality of fluorescently-labeled detection reagents includes, for each respective genomic locus in the second set of two or more genomic loci:
a third detection reagent that is specific for the presence of a first allele at the respective genomic locus, wherein the first allele is the most prevalent allele in a population of the species of the subject and the third detection reagent is labeled with a third fluorescent moiety, and
a fourth detection reagent that is specific for the presence of a second allele at the respective genomic locus, wherein the second allele is a minor allele in the population and the fourth detection reagent is labeled with a fourth fluorescent moiety that is distinguishable from the third fluorescent moiety
v) during or after the amplifying iv), detecting a fluorescent signal corresponding to the third fluorescent moiety and the fourth fluorescent moiety in the second single reaction vessel; and
vi) responsive to the detecting v):
when the contribution of the fourth fluorescent moiety to the fluorescent signal detected in the reaction vessel does not satisfy a threshold contribution, determining that the subject is homozygous for the first allele at each respective loci in the second set of two or more loci, and
when the contribution of the fourth fluorescent moiety to the fluorescent signal detected in the reaction vessel satisfies the threshold contribution, performing a second plurality of secondary allele detection assays, wherein each secondary allele detection assay in the second plurality of secondary allele detection assays determines the allele status at one respective genomic locus in the second set of two or more genomic loci, thereby determining the allele status at each respective loci in the second set of two or more genomic loci.

215. The method of claim 214, wherein the second set of two or more genomic loci comprises one of:
the human alleles corresponding to the SNPs rs5030863, rs28371685, and rs5030867,
the human alleles corresponding to the SNPs rs17884712, rs72552267, and rs72558187, or
the human alleles corresponding to the SNPs rs5030862 and rs56337013.

216. The method of claim 210 or 215, wherein the determining a) further comprises:
vii) amplifying, in a third single in vitro reaction vessel, a third set of two or more genomic loci in the plurality of genomic loci, by polymerase chain reaction (PCR), from nucleic acids isolated from a sample obtained from the subject, in the presence of a plurality of fluorescently-labeled detection reagents, wherein the plurality of fluorescently-labeled detection reagents includes, for each respective genomic locus in the third set of two or more genomic loci:
a fifth detection reagent that is specific for the presence of a first allele at the respective genomic locus, wherein the first allele is the most prevalent allele in a population of the species of the subject and the fifth detection reagent is labeled with a third fluorescent moiety, and
a sixth detection reagent that is specific for the presence of a second allele at the respective genomic locus, wherein the second allele is a minor allele in the population and the sixth detection reagent is labeled with a sixth fluorescent moiety that is distinguishable from the fifth fluorescent moiety
viii) during or after the amplifying vii), detecting a fluorescent signal corresponding to the fifth fluorescent moiety and the sixth fluorescent moiety in the third single reaction vessel; and
ix) responsive to the detecting viii):
when the contribution of the sixth fluorescent moiety to the fluorescent signal detected in the reaction vessel does not satisfy a threshold contribution, determining that the subject is homozygous for the first allele at each respective loci in the second set of two or more loci, and
when the contribution of the sixth fluorescent moiety to the fluorescent signal detected in the reaction vessel satisfies the threshold contribution, performing a third plurality of secondary allele detection assays, wherein each secondary allele detection assay in the third plurality of secondary allele detection assays determines the allele status at one respective genomic locus in the third set of two or more genomic loci, thereby determining the allele status at each respective loci in the third set of two or more genomic loci.

217. The method of claim 216, wherein the first, second, and third set of two or more genomic loci comprise:
the human alleles corresponding to the SNPs rs5030863, rs28371685, and rs5030867,
the human alleles corresponding to the SNPs rs17884712, rs72552267, and rs72558187, and
the human alleles corresponding to the SNPs rs5030862 and rs56337013, respectively.

218. The method of any one of claims 210-217, wherein:
the plurality of genomic loci comprises the human allele corresponding to the rs7997012 SNP, and
when the subject is determined to carry the rs7997012 SNP, the one or more recommendations include assigning antidepressant therapy comprising administration of a selective serotonin reuptake inhibitor (SSRI).

219. The method of claim 218, wherein the SSRI is citalopram.

220. The method of any one of claims 210-219, wherein:
the plurality of genomic loci comprises the human allele corresponding to the rs3813929 SNP, and
when the subject is determined to carry the rs3813929 SNP, the one or more recommendations include assigning antidepressant therapy comprising administering a low dose of an antipsychotics.

221. The method of claim 220, wherein the antipsychotics is amisulpride, clozapine, haloperidol, iloperidone, olanzapine, quetiapine, risperidone, or ziprasidone.

222. The method of any one of claims 210-221, wherein:
the plurality of genomic loci comprises the human allele corresponding to the rs1045642 SNP, and
when the subject is determined to carry the rs1045642 SNP, the one or more recommendations include assigning antidepressant therapy comprising administration of a low dose of an antipsychotics.

223. The method of claim 222, wherein the antipsychotics is chlorpromazine.

224. The method of any one of claims 210-223, wherein:
the plurality of genomic loci comprises the human allele corresponding to the rs2032583 SNP, and
when the subject is determined to carry the rs2032583 SNP, the one or more recommendations include assigning antidepressant therapy comprising administration of a SSRI or a tricyclic antidepressant (TCA).

225. The method of claim 224, wherein the SSRI is citalopram, fluvoxamine, paroxetine, or sertraline.

226. The method of claim 224, wherein the TCA is amitriptyline.

227. The method of any one of claims 210-226, wherein:
the plurality of genomic loci comprises the human allele corresponding to the rs1800544 SNP, and
when the subject is determined to carry the rs1800544 SNP, the one or more recommendations include assigning antidepressant therapy comprising administration of a serotonin-norepinephrine reuptake inhibitor (SNRI).

228. The method of claim 227, wherein the SNRI is milnacipran.

229. The method of any one of claims 210-228, wherein:
the plurality of genomic loci comprises the human allele corresponding to the rs10994336 SNP, and
when the subject is determined to carry the rs10994336 SNP, the one or more recommendations include administering sodium channel modulating agents.

230. The method of claim 229, wherein the sodium channel modulating agent is lamotrigine.

231. The method of any one of claims 210-229, wherein:
the plurality of genomic loci comprises the human allele corresponding to the rs6265 SNP, and
when the subject is determined to carry the rs6265 SNP, the one or more recommendations include assigning antidepressant therapy comprising administration of a SSRI or an antipsychotics.

232. The method of claim 231, wherein the SSRI is paroxetine.

233. The method of claim 231, wherein the antipsychotics is clozapine.

234. The method of any one of claims 210-233, wherein:
the plurality of genomic loci comprises the human allele corresponding to the rs1006737 SNP, and
when the subject is determined to carry the rs1006737 SNP, the one or more recommendations include assigning antidepressant therapy comprising administering a low dose of SSRI.

235. The method of claim 234, wherein the SSRI is citalopram.

236. The method of any one of claims 210-235, wherein:
the plurality of genomic loci comprises the human allele corresponding to the rs4680 SNP, and
when the subject is determined to carry the rs4680 SNP, the one or more recommendations include assigning antidepressant therapy comprising administration of a SSRI or a SNRI.

237. The method of claim 236, wherein the SSRI is paroxetine.

238. The method of claim 236, wherein the SNRI is venlafaxine.

239. The method of any one of claims 210-238, wherein:
the plurality of genomic loci comprises the human allele corresponding to the rs2470890 SNP, and
when the subject is determined to carry the rs2470890 SNP, the one or more recommendations include assigning antidepressant therapy comprising administering a low dose of antipsychotics.

240. The method of claim 239, wherein the antipsychotics is clozapine.

241. The method of any one of claims 210-239, wherein:
the plurality of genomic loci comprises the human allele corresponding to the rs2069514 SNP, and
when the subject is determined to carry the rs2069514 SNP, the one or more recommendations include assigning antidepressant therapy comprising administration of a low dose of antipsychotics.

242. The method of any one of claims 210-241, wherein:
the plurality of genomic loci comprises the human allele corresponding to the rs35694136 SNP, and
when the subject is determined to carry the rs35694136 SNP, the one or more recommendations include assigning antidepressant therapy comprising administration of a low dose of antipsychotics.

243. The method of any one of claims 210-242, wherein:
the plurality of genomic loci comprises the human allele corresponding to the rs2069526 SNP, and
when the subject is determined to carry the rs2069526 SNP, the one or more recommendations include assigning antidepressant therapy comprising administering a low dose of a SSRI.

244. The method of claim 243, wherein the SSRI is escitalopram.

245. The method of any one of claims 210-244, wherein:
the plurality of genomic loci comprises the human allele corresponding to the rs762551 SNP, and
when the subject is determined to carry the rs762551 SNP, the one or more recommendations include assigning antidepressant therapy comprising administration of a high dose of a SSRI.

246. The method of claim 245, wherein the SSRI is paroxetine.

247. The method of any one of claims 210-246, wherein:
the plurality of genomic loci comprises the human allele corresponding to the rs72547513 SNP, and
when the subject is determined to carry the rs72547513 SNP, the one or more recommendations include administering a low dose of pharmaceutical agents that are normally metabolized by cytochrome P450 1A2.

248. The method of any one of claims 210-247, wherein:
the plurality of genomic loci comprises the human allele corresponding to the rs2279343 SNP, and
when the subject is determined to carry the rs2279343 SNP, the one or more recommendations include assigning therapy for substance abuse.

249. The method of claim 248, wherein the substance is heroin, and
the therapy comprises administering a high dose of methadone.

250. The method of claim 248, wherein the substance is nicotine, and
the therapy comprises administering bupropion.

251. The method of any one of claims 210-250, wherein:
the plurality of genomic loci comprises the human allele corresponding to the rs3211371 SNP, and
when the subject is determined to carry the rs3211371 SNP, the one or more recommendations include assigning non-heroin therapy comprising administration of a high dose of methadone.

252. The method of any one of claims 210-251, wherein:
the plurality of genomic loci comprises the human allele corresponding to the rs3745274 SNP, and
when the subject is determined to carry the rs3745274 SNP, the one or more recommendations include assigning therapy for substance abuse.

253. The method of claim 252, wherein the substance is heroin, and
the therapy comprises administering a high dose of methadone.

254. The method of claim 252, wherein the substance is nicotine, and
the therapy comprises administering bupropion.

255. The method of any one of claims 210-254, wherein:
the plurality of genomic loci comprises the human allele corresponding to the rs2279343 SNP, and
when the subject is determined to carry the rs2279343 SNP, the one or more recommendations include assigning therapy for substance abuse.

256. The method of claim 255, wherein the substance is heroin, and
the therapy comprises administering a high dose of methadone.

257. The method of claim 255, wherein the substance is nicotine, and
the therapy comprises administering bupropion.

258. The method of any one of claims 210-257, wherein:
the plurality of genomic loci comprises the human allele corresponding to the rs2279343 SNP, and
when the subject is determined to carry the rs2279343 SNP, the one or more recommendations include assigning antidepressant therapy comprising administration of mirtazapine.

259. The method of any one of claims 210-258, wherein:
the plurality of genomic loci comprises the human allele corresponding to the rs4244285 SNP, and
when the subject is determined to carry the rs4244285 SNP, the one or more recommendations include assigning antidepressant therapy comprising administration of a low dose of a TCA or a SSRI.

260. The method of claim 259, wherein the TCA is amitriptyline.

261. The method of claim 259, wherein the SSRI is citalopram or escitalopram.

262. The method of any one of claims 210-261, wherein:
the plurality of genomic loci comprises the human allele corresponding to the rs17878459 SNP, and
when the subject is determined to carry the rs17878459 SNP, the one or more recommendations include assigning antidepressant therapy comprising administering a low dose of antipsychotics.

263. The method of any one of claims 210-262, wherein:
the plurality of genomic loci comprises the human allele corresponding to the rs4986893 SNP, and
when the subject is determined to carry the rs4986893 SNP, the one or more recommendations include assigning antidepressant therapy comprising administering a low dose of a SSRI.

264. The method of claim 263, wherein the SSRI is citalopram or escitalopram.

265. The method of any one of claims 210-264, wherein:
the plurality of genomic loci comprises the human allele corresponding to the rs57081121 SNP, and
when the subject is determined to carry the rs57081121 SNP, the one or more recommendations include assigning antidepressant therapy comprising administering a low dose of a SSRI.

266. The method of claim 265, wherein the SSRI is citalopram or escitalopram.

267. The method of any one of claims 210-266, wherein:
the plurality of genomic loci comprises the human allele corresponding to the rs28399504 SNP, and
when the subject is determined to carry the rs28399504 SNP, the one or more recommendations include assigning antidepressant therapy comprising administration of a low dose of a SSRI.

268. The method of claim 267, wherein the SSRI is citalopram or escitalopram.

269. The method of any one of claims 210-268, wherein:
the plurality of genomic loci comprises the human allele corresponding to the rs56337013 SNP, and
when the subject is determined to carry the rs56337013 SNP, the one or more recommendations include administering a low dose of a pharmaceutical agent that is metabolized by cytochrome P450 2C19 (CYP2C19) when the subject is determined to carry the rs56337013 SNP.

270. The method of any one of claims 210-269, wherein:
the plurality of genomic loci comprises the human allele corresponding to the rs72552267 SNP, and
when the subject is determined to carry the rs72552267 SNP, the one or more recommendations include administering a low dose of a pharmaceutical agent that is metabolized by cytochrome P450 2C19 (CYP2C19) when the subject is determined to carry the rs72552267 SNP.

271. The method of any one of claims 210-270, wherein:
the plurality of genomic loci comprises the human allele corresponding to the rs72558186 SNP, and
when the subject is determined to carry the rs72558186 SNP, the one or more recommendations include administering a low dose of a pharmaceutical agent that is metabolized by cytochrome P450 2C19 (CYP2C19).

272. The method of any one of claims 210-271, wherein:
the plurality of genomic loci comprises the human allele corresponding to the rs41291556 SNP, and
when the subject is determined to carry the rs41291556 SNP, the one or more recommendations include administering a low dose of a pharmaceutical agent that is metabolized by cytochrome P450 2C19 (CYP2C19).

273. The method of any one of claims 210-272, wherein:
the plurality of genomic loci comprises the human allele corresponding to the rs17884712 SNP, and
when the subject is determined to carry the rs17884712 SNP, the one or more recommendations include administering a low dose of a pharmaceutical agent that is metabolized by cytochrome P450 2C19 (CYP2C19) when the subject is determined to carry the rs17884712 SNP.

274. The method of any one of claims 210-273, wherein:
the plurality of genomic loci comprises the human allele corresponding to the rs6413438 SNP, and
when the subject is determined to carry the rs6413438 SNP, the one or more recommendations include administering a low dose of a pharmaceutical agent that is metabolized by cytochrome P450 2C19 (CYP2C19).

275. The method of any one of claims 210-274, wherein:
the plurality of genomic loci comprises the human allele corresponding to the rs12248560 SNP, and
when the subject is determined to carry the rs12248560 SNP, the one or more recommendations include assigning antidepressant therapy comprising administration of a low dose of a SSRI.

276. The method of claim 275, wherein the SSRI is citalopram or escitalopram.

277. The method of any one of claims 210-276, wherein:
the plurality of genomic loci comprises the human allele corresponding to the rs12248560 SNP, and
when the subject is determined to carry the rs12248560 SNP, the one or more recommendations include assigning antidepressant therapy comprising administration of a low dose of a TCA.

278. The method of claim 277, wherein the TCA is amitriptyline or clomipramine.

279. The method of any one of claims 210-278, wherein:
the plurality of genomic loci comprises the human allele corresponding to the rs12769205 SNP, and
when the subject is determined to carry the rs12769205 SNP, the one or more recommendations include assigning antidepressant therapy comprising administration of a low dose of a SSRI or a TCA.

280. The method of claim 279, wherein the SSRI is sertraline or escitalopram.

281. The method of claim 279, wherein the TCA is amitriptyline or imipramine.

282. The method of any one of claims 210-281, wherein:
the plurality of genomic loci comprises the human allele corresponding to the rs3758581 SNP, and
when the subject is determined to carry the rs3758581 SNP, the one or more recommendations include assigning antidepressant therapy comprising administration of a low dose of a SSRI or a TCA.

283. The method of claim 282, wherein the SSRI is sertraline or escitalopram.

284. The method of claim 282, wherein the TCA is amitriptyline or imipramine.

285. The method of any one of claims 210-284, wherein:
the plurality of genomic loci comprises the human allele corresponding to the rs1799853 SNP, and
when the subject is determined to carry the rs1799853 SNP, the one or more recommendations include assigning psychotropic therapy comprising administration of a low dose of valproic acid.

286. The method of any one of claims 210-285, wherein:
the plurality of genomic loci comprises the human allele corresponding to the rs1057910 SNP, and
when the subject is determined to carry the rs1057910 SNP, the one or more recommendations include assigning psychotropic therapy comprising administration of a low dose of a TCA or valproic acid.

287. The method of claim 286, wherein the TCA is trimipramine or doxepin.

288. The method of any one of claims 210-287, wherein:
the plurality of genomic loci comprises the human allele corresponding to the rs56165452 SNP, and

289. when the subject is determined to carry the rs56165452 SNP, the one or more recommendations include administering low dose of a pharmaceutical agent that is metabolized by cytochrome P450 2C9. The method of any one of claims 210-288, wherein:
the plurality of genomic loci comprises the human allele corresponding to the rs28371686 SNP, and
when the subject is determined to carry the rs28371686 SNP, the one or more recommendations include administering low dose of a pharmaceutical agent that is metabolized by cytochrome P450 2C9.

290. The method of any one of claims 210-289, wherein:
the plurality of genomic loci comprises the human allele corresponding to the rs9332131 SNP, and
when the subject is determined to carry the rs9332131 SNP, the one or more recommendations include administering a low dose of an antiepileptic drug (AED).

291. The method of claim 290, wherein the AED is phenytoin.

292. The method of any one of claims 210-291, wherein:
the plurality of genomic loci comprises the human allele corresponding to the rs7900194 SNP, and
when the subject is determined to carry the rs7900194 SNP, the one or more recommendations include administering a low dose of an antiepileptic drug (AED).

293. The method of claim 292, wherein the AED is phenytoin.

294. The method of any one of claims 210-293, wherein:
the plurality of genomic loci comprises the human allele corresponding to the rs28371685 SNP, and
when the subject is determined to carry the rs28371685 SNP, the one or more recommendations include administering a low dose of a pharmaceutical agent that is metabolized by cytochrome P450 2C9 (CYP2C9) when the subject is determined to carry the rs28371685 SNP.

295. The method of any one of claims 210-294, wherein:
the plurality of genomic loci comprises the human allele corresponding to the rs72558187 SNP, and
when the subject is determined to carry the rs72558187 SNP, the one or more recommendations include administering a low dose of an antiepileptic drug (AED).

296. The method of claim 295, wherein the AED is phenytoin.

297. The method of any one of claims 210-296, wherein:
the plurality of genomic loci comprises the human allele corresponding to the rs1135840 SNP, and
when the subject is determined to carry the rs1135840 SNP, the one or more recommendations include assigning antidepressant therapy comprising administration of a TCA, a SSRI, or a norepinephrine reuptake inhibitor (NRI).

298. The method of claim 297, wherein the TCA is imipramine, amitriptyline, trimipramine, clomipramine, desipramine, or doxepin.

299. The method of claim 297, wherein the SSRI is paroxetine, citalopram, or escitalopram.

300. The method of claim 297, wherein the NRI is atomoxetine.

301. The method of any one of claims 210-300, wherein:
the plurality of genomic loci comprises the human allele corresponding to the rs16947 SNP, and
when the subject is determined to carry the rs16947 SNP, the one or more recommendations include assigning antidepressant therapy comprising administration of a TCA, a SSRI, or a norepinephrine reuptake inhibitor (NRI).

302. The method of claim 301, wherein the TCA is imipramine, amitriptyline, trimipramine, clomipramine, desipramine, or doxepin.

303. The method of claim 301, wherein the SSRI is paroxetine, citalopram, or escitalopram.

304. The method of claim 301, wherein the NRI is atomoxetine.

305. The method of any one of claims 210-304, wherein:
the plurality of genomic loci comprises the human allele corresponding to the rs1135824 SNP, and
when the subject is determined to carry the rs1135824 SNP, the one or more recommendations include assigning antidepressant therapy comprising administration of a low dose of a SSRI, a TCA or a NRI.

306. The method of claim 305, wherein the SSRI is paroxetine, citalopram, fluvoxamine, fluoxetine, or escitalopram.

307. The method of claim 305, wherein the TCA is imipramine, amitriptyline, trimipramine, clomipramine, desipramine, doxepin, trimipramine, or nortriptyline.

308. The method of claim 305, wherein the NRI is atomoxetine.

309. The method of any one of claims 210-308, wherein:
the plurality of genomic loci comprises the human allele corresponding to the rs35742686 SNP, and
when the subject is determined to carry the rs35742686 SNP, the one or more recommendations include assigning antidepressant therapy comprising administration of a low dose of a SSRI, a TCA or a NRI.

310. The method of claim 309, wherein the SSRI is paroxetine, citalopram, fluvoxamine, fluoxetine, or escitalopram.

311. The method of claim 309, wherein the TCA is imipramine, amitriptyline, trimipramine, clomipramine, desipramine, doxepin, trimipramine, or nortriptyline.

312. The method of claim 309, wherein the NRI is atomoxetine.

313. The method of any one of claims 210-312, wherein:
the plurality of genomic loci comprises the human allele corresponding to the rs3892097 SNP, and
when the subject is determined to carry the rs3892097 SNP, the one or more recommendations include assigning antidepressant therapy comprising administration of a low dose of a SSRI, a TCA, or a NRI.

314. The method of claim 313, wherein the SSRI is paroxetine, citalopram, fluvoxamine, fluoxetine, or escitalopram.

315. The method of claim 313, wherein the TCA is imipramine, amitriptyline, trimipramine, clomipramine, desipramine, doxepin, trimipramine, or nortriptyline.

316. The method of claim 313, wherein the NRI is atomoxetine.

317. The method of any one of claims 210-316, wherein:
the plurality of genomic loci comprises the human allele corresponding to the rs5030655 SNP, and
when the subject is determined to carry the rs5030655 SNP, the one or more recommendations include assigning antidepressant therapy comprising administration of a low dose of a SSRI, a TCA, or a NRI.

318. The method of claim 317, wherein the SSRI is paroxetine, citalopram, fluvoxamine, fluoxetine, or escitalopram.

319. The method of claim 317, wherein the TCA is imipramine, amitriptyline, trimipramine, clomipramine, desipramine, doxepin, trimipramine, or nortriptyline.

320. The method of claim 317, wherein the NRI is atomoxetine.

321. The method of any one of claims 210-320, wherein:
the plurality of genomic loci comprises the human allele corresponding to the rs5030867 SNP, and
when the subject is determined to carry the rs5030867 SNP, the one or more recommendations include administering a low dose of an antipsychotics when the subject is determined to carry the rs5030867 SNP.

322. The method of any one of claims 210-321, wherein:
the plurality of genomic loci comprises the human allele corresponding to the rs5030865 SNP, and
when the subject is determined to carry the rs5030865 SNP, the one or more recommendations include assigning antidepressant therapy comprising administering a low dose of an antipsychotics.

323. The method of claim 322, wherein the antipsychotics is risperidone.

324. The method of any one of claims 210-323, wherein:
the plurality of genomic loci comprises the human allele corresponding to the rs5030656 SNP, and
when the subject is determined to carry the rs5030656 SNP, the one or more recommendations include administering a low dose of a pharmaceutical agent that is metabolized by cytochrome P450 2D6.

325. The method of any one of claims 210-324, wherein:
the plurality of genomic loci comprises the human allele corresponding to the rs1065852 SNP, and
when the subject is determined to carry the rs1065852 SNP, the one or more recommendations include assigning antidepressant therapy comprising administration of a low dose of a SSRI, a TCA, a NRI or an antipsychotics.

326. The method of claim 325, wherein the SSRI is paroxetine, citalopram, fluvoxamine, fluoxetine, or escitalopram.

327. The method of claim 325, wherein the TCA is imipramine, amitriptyline, trimipramine, clomipramine, desipramine, doxepin, or nortriptyline.

328. The method of claim 325, wherein the NRI is atomoxetine.

329. The method of any one of claims 210-328, wherein:
the plurality of genomic loci comprises the human allele corresponding to the rs5030863 SNP, and
when the subject is determined to carry the rs5030863 SNP, the one or more recommendations include administering a low dose of a pharmaceutical agent that is metabolized by cytochrome P450 2D6 (CYP2D6) when the subject is determined to carry the rs5030863 SNP.

330. The method of any one of claims 210-329, wherein:
the plurality of genomic loci comprises the human allele corresponding to the rs5030862 SNP, and
when the subject is determined to carry the rs5030862 SNP, the one or more recommendations include administering a low dose of a pharmaceutical agent that is metabolized by cytochrome P450 2D6 (CYP2D6) when the subject is determined to carry the rs5030862 SNP.

331. The method of any one of claims 210-330, wherein:
the plurality of genomic loci comprises the human allele corresponding to the rs5030865(T) SNP, and
when the subject is determined to carry the rs5030865(T) SNP, the one or more recommendations include assigning antidepressant therapy comprising administration of a low dose of an antipsychotics.

332. The method of claim 331, wherein the antipsychotics is risperidone.

333. The method of any one of claims 210-332, wherein:
the plurality of genomic loci comprises the human allele corresponding to the rs774671100 SNP, and
when the subject is determined to carry the rs774671100 SNP, the one or more recommendations include administering a low dose of pharmaceutical agents that are normally metabolized by cytochrome P450 2D6.

334. The method of any one of claims 210-333, wherein:
the plurality of genomic loci comprises the human allele corresponding to the rs28371706 SNP, and
when the subject is determined to carry the rs28371706 SNP, the one or more recommendations include assigning antidepressant therapy comprising administration of a TCA.

335. The method of claim 334, wherein the TCA is haloperidol.

336. The method of claim 334, wherein the TCA is desipramine or nortriptyline, and a low dose of the TCA is administered.

337. The method of any one of claims 210-336, wherein:
the plurality of genomic loci comprises the human allele corresponding to the rs16947 SNP, and
when the subject is determined to carry the rs16947 SNP, the one or more recommendations include assigning antidepressant therapy comprising administration of a TCA.

338. The method of claim 337, wherein the TCA is haloperidol.

339. The method of claim 337, wherein the TCA is desipramine or nortriptyline, and a low dose of the TCA is administered.

340. The method of any one of claims 210-339, wherein:
the plurality of genomic loci comprises the human allele corresponding to the rs61736512 SNP, and
when the subject is determined to carry the rs61736512 SNP, the one or more recommendations include administering a low dose of a pharmaceutical agent that is metabolized by cytochrome P450 2D6 (CYP2D6).

341. The method of any one of claims 210-340, wherein:
the plurality of genomic loci comprises the human allele corresponding to the rs1058164 SNP, and
when the subject is determined to carry the rs1058164 SNP, the one or more recommendations include administering a low dose of a pharmaceutical agent that is metabolized by cytochrome P450 2D6 (CYP2D6).

342. The method of any one of claims 210-341, wherein:
the plurality of genomic loci comprises the human allele corresponding to the rs59421388 SNP, and
when the subject is determined to carry the rs59421388 SNP, the one or more recommendations include administering a low dose of a pharmaceutical agent that is metabolized by cytochrome P450 2D6 (CYP2D6).

343. The method of any one of claims 210-342, wherein:
the plurality of genomic loci comprises the human allele corresponding to the rs28371725 SNP, and
when the subject is determined to carry the rs28371725 SNP, the one or more recommendations include assigning antidepressant therapy comprising administration of a TCA, a SSRI or a SNRI.

344. The method of claim 343, wherein the SSRI is citalopram or escitalopram.

345. The method of claim 343, wherein the TCA is desipramine, aripiprazole, haloperidol, levomepromazine, quetiapine, or risperidone, and a low dose of the TCA is administered.

346. The method of claim 343, wherein the SSRI is desipramine, aripiprazole, haloperidol, levomepromazine, or quetiapine, and a low dose of the SSRI is administered.

347. The method of any one of claims 210-346, wherein:
the plurality of genomic loci comprises the human allele corresponding to the rs35599367 SNP, and
when the subject is determined to carry the rs35599367 SNP, the one or more recommendations include assigning antidepressant therapy comprising administration of a low dose of an antipsychotics.

348. The method of claim 347, wherein the antipsychotics is risperidone.

349. The method of any one of claims 210-348, wherein:
the plurality of genomic loci comprises the human allele corresponding to the rs776746 SNP, and
when the subject is determined to carry the rs776746 SNP, the one or more recommendations include administering a low dose of pharmaceutical agents that are normally metabolized by cytochrome P450 3A5.

350. The method of any one of claims 210-349, wherein:
the plurality of genomic loci comprises the human allele corresponding to the rs10264272 SNP, and
when the subject is determined to carry the rs10264272 SNP, the one or more recommendations include administering a low dose of pharmaceutical agents that are normally metabolized by cytochrome P450 3A5.

351. The method of any one of claims 210-350, wherein:
the plurality of genomic loci comprises the human allele corresponding to the rs41303343 SNP, and
when the subject is determined to carry the rs41303343 SNP, the one or more recommendations include administering a low dose of pharmaceutical agents that are normally metabolized by cytochrome P450 3A5.

352. The method of any one of claims 210-351, wherein:
the plurality of genomic loci comprises the human allele corresponding to the rs1799732 SNP, and
when the subject is determined to carry the rs1799732 SNP, the one or more recommendations include assigning antidepressant therapy comprising administration of a antipsychotics.

353. The method of claim 352, wherein the antipsychotics is aripiprazole, bromperidol, chlorpromazine, clozapine, nemonapride, olanzapine, or risperidone.

354. The method of claim 352, wherein the antipsychotics is risperidone, and
a low dose of the antipsychotics is administered.

355. The method of any one of claims 210-354, wherein:
the plurality of genomic loci comprises the human allele corresponding to the rs1799732 SNP, and
when the subject is determined to carry the rs1799732 SNP, the one or more recommendations include assigning therapy for tobacco use disorder comprising administration of a non-nicotine replacement.

356. The method of any one of claims 210-355, wherein:
the plurality of genomic loci comprises the human allele corresponding to the rs2832407 SNP, and
when the subject is determined to carry the rs2832407 SNP, the one or more recommendations include assigning therapy for alcohol abuse comprising administration of a low dose of topiramate.

357. The method of any one of claims 210-356, wherein:
the plurality of genomic loci comprises the human allele corresponding to the rs1061235 SNP, and
when the subject is determined to carry the rs1061235 SNP, the one or more recommendations include administering a lose dose of an anticonvulsant or an AED.

358. The method of claim 357, wherein the anticonvulsant is carbamazepine, oxcarbazepine, or lamotrigine.

359. The method of claim 357, wherein the AED is phenytoin.

360. The method of any one of claims 210-359, wherein:
the plurality of genomic loci comprises the human allele corresponding to the rs2395148 SNP, and
when the subject is determined to carry the rs2395148 SNP, the one or more recommendations include administering a lose dose of an anticonvulsant or an AED.

361. The method of claim 360, wherein the anticonvulsant is carbamazepine, oxcarbazepine, or lamotrigine.

362. The method of claim 360, wherein the AED is phenytoin.

363. The method of any one of claims 210-362, wherein:
the plurality of genomic loci comprises the human allele corresponding to the rs489693 SNP, and
when the subject is determined to carry the rs489693 SNP, the one or more recommendations include assigning antidepressant therapy comprising administration of a low dose of an antipsychotics.

364. The method of claim 363, wherein the antipsychotics is amisulpride, aripiprazole, clozapine, haloperidol, olanzapine, paliperidone, quetiapine, risperidone, or ziprasidone.

365. The method of any one of claims 210-364, wherein:
the plurality of genomic loci comprises the human allele corresponding to the rs1801131 SNP, and
when the subject is determined to carry the rs1801131 SNP, the one or more recommendations include assigning antidepressant therapy comprising administration of a low dose of antipsychotics.

366. The method of claim 365, wherein the antipsychotics is olanzapine or clozapine.

367. The method of any one of claims 210-366, wherein:
the plurality of genomic loci comprises the human allele corresponding to the rs1801131 SNP, and
when the subject is determined to carry the rs1801131 SNP, the one or more recommendations include assigning anti-depression therapy comprising administration of 1-methylfolate or vitamin B-complex.

368. The method of any one of claims 210-367, wherein:
the plurality of genomic loci comprises the human allele corresponding to the rs1801133 SNP, and
when the subject is determined to carry the rs1801133 SNP, the one or more recommendations include assigning antidepressant therapy comprising administration of a low dose of antipsychotics.

369. The method of any one of claims 210-368, wherein:
the plurality of genomic loci comprises the human allele corresponding to the rs1801133 SNP, and
when the subject is determined to carry the rs1801133 SNP, the one or more recommendations include assigning anti-depression therapy comprising administration of 1-methylfolate or vitamin B-complex.

370. The method of any one of claims 210-369, wherein:
the plurality of genomic loci comprises the human allele corresponding to the rs1801133 SNP, and
when the subject is determined to carry the rs1801133 SNP, the one or more recommendations include assigning therapy for cocaine abuse comprising administration of disulfiram.

371. The method of any one of claims 210-370, wherein:
the plurality of genomic loci comprises the human allele corresponding to the rs1799971 SNP, and
when the subject is determined to carry the rs1799971 SNP, the one or more recommendations include assigning therapy for substance abuse.

372. The method of claim 371, wherein the substance is tobacco, and
the therapy comprises administering a nicotine-replacement.

373. The method of claim 371, wherein the substance is opioid, and
the therapy comprises administering a low dose of methadone.

374. The method of any one of claims 210-373, wherein:
the plurality of genomic loci comprises the human allele corresponding to the rs25531 SNP, and
when the subject is determined to carry the rs25531 SNP, the one or more recommendations include assigning antidepressant therapy comprising administration of a low dose of SSRI.

375. The method of claim 202, wherein the SSRI is fluoxetine or citalopram.

376. The method of any one of claims 210-375, wherein:
the plurality of genomic loci comprises the human allele corresponding to the rs63749047 SNP, and
when the subject is determined to carry the rs63749047 SNP, the one or more recommendations include assigning antidepressant therapy comprising administration of a low dose of SSRI.

377. The method of claim 376, wherein the SSRI is citalopram, escitalopram, fluoxetine, fluvoxamine, paroxetine, or sertraline.

378. The method of any one of claims 210-377, wherein:
the plurality of genomic loci comprises the human allele corresponding to the rs2011425 SNP, and
when the subject is determined to carry the rs2011425 SNP, the one or more recommendations include administering a low dose of lamotrigine, asenapine, or trifluoperazine.

379. The method of any one of claims 210-378, wherein:
the plurality of genomic loci comprises the human allele corresponding to the rs1902023 SNP, and
when the subject is determined to carry the rs1902023 SNP, the one or more recommendations include assigning psychotropic therapy comprising administration of a low dose of a benzodiazepine (BZD).

380. The method of claim 379, wherein the BZD is clonazepam, diazepam, lorazepam, oxazepam, or temazepam.

381. The method of claim 210, wherein the treatment guidance is for bipolar disorder and the ICD-10 code is F31.0 (bipolar disorder, current episode hypomanic), F31.1 (bipolar disorder, current episode manic without psychotic features), F31.2 (bipolar disorder, current episode manic severe with psychotic features), F31.3 (bipolar disorder, current episode depressed, mild or moderate severity), F31.5 (bipolar disorder, current episode depressed, severe, with psychotic features), F31.6 (bipolar disorder, current episode mixed), F31.7 (bipolar disorder, currently in remission), F31.8 (other bipolar disorders), or F31.9 (bipolar disorder, unspecified).

382. The method of claim 210, wherein the treatment guidance is for major depressive disorder, single episode and the ICD-10 code is F32.0 (major depressive disorder, single episode, mild), F32.1 (major depressive disorder, single episode, moderate), F32.2 (major depressive disorder, single episode, severe without psychotic features), F32.3 (major depressive disorder, single episode, severe with psychotic features), F32.4 (major depressive disorder, single episode, in partial remission), F32.5 (major depressive disorder, single episode, in full remission), F32.8 (other depressive episodes), F32.9 (major depressive disorder, single episode, unspecified), F33.0 (major depressive disorder, recurrent, mild), F33.1 (major depressive disorder, recurrent, moderate), F33.2 (major depressive disorder, recurrent severe without psychotic features), F33.3 (major depressive disorder, recurrent, severe with psychotic symptoms), F33.4 (major depressive disorder, recurrent, in remission), F32.4 (major depressive disorder, single episode, in partial remission), F33.8 (other recurrent depressive disorders), or F33.9 (major depressive disorder, recurrent, unspecified).

383. The method of claim 210, wherein the treatment guidance is for anxiety disorder and the ICD-10 code is F40.0 (agoraphobia), F40.1 (social phobias), F40.2 (specific (isolated) phobias), F40.8 (other phobic anxiety disorders), F40.9 (phobic anxiety disorder, unspecified), F41.0 (panic disorder (episodic paroxysmal anxiety)), F41.1 (generalized anxiety disorder), F41.3 (other mixed anxiety disorders), F41.8 (other specified anxiety disorders), or F41.9 (anxiety disorder, unspecified).

384. The method of claim 210, wherein the treatment guidance is for obsessive-compulsive disorder and the ICD-10 code is F42.2 (mixed obsessional thoughts and acts), F42.3 (hoarding disorder), F42.4 (excoriation (skin-picking) disorder), F42.8 (other obsessive-compulsive disorder), F42.9 (obsessive-compulsive disorder, unspecified), or F60.5 (obsessive-compulsive personality disorder).

385. The method of claim 210, wherein the treatment guidance is for attention-deficit hyperactivity disorder and the ICD-10 code is F90.0 (attention-deficit hyperactivity disorder, predominantly inattentive type), F90.1 (attention-deficit hyperactivity disorder, predominantly hyperactive type), F90.2 (attention-deficit hyperactivity disorder, combined type), F90.8 (attention-deficit hyperactivity disorder, other type), or F90.9 (attention-deficit hyperactivity disorder, unspecified type).

386. The method of claim 210, wherein the treatment guidance is for post-traumatic stress disorder and the ICD-10 code is F43.1 (post-traumatic stress disorder (PTSD)).

387. The method of claim 210, wherein the treatment guidance is for autistic disorder and the ICD-10 code is F84.0 (autistic disorder).

388. The method of claim 210, wherein the treatment guidance is for schizophrenia and the ICD-10 code is F20.0 (paranoid schizophrenia), F20.1 (disorganized schizophrenia) F20.2 (catatonic schizophrenia) F20.3 (undifferentiated schizophrenia) F20.5 (residual schizophrenia), F20.8 (other schizophrenia), or F20.9 (schizophrenia, unspecified).

389. The method of claim 210, wherein the treatment guidance is for personality disorder and the ICD-10 code is F60.0 (paranoid personality disorder), F60.1 (schizoid personality disorder), F60.2 (antisocial personality disorder), F60.3 (borderline personality disorder), F60.4 (histrionic personality disorder), F60.5 (obsessive-compulsive personality disorder), F60.6 (avoidant personality disorder), F60.7 (dependent personality disorder), F60.8 (other specific personality disorders), F60.9 (personality disorder, unspecified), F07.0 (personality change due to known physiological condition), F07.8 (other personality and behavioral disorders due to known physiological condition), or F07.9 (unspecified personality and behavioral disorder due to known physiological condition).

390. The method of claim 210, wherein the treatment guidance is for chronic pain and the ICD-10 code is G89.2 (chronic pain, not elsewhere classified), or G89.4 (chronic pain syndrome).

391. The method of claim 210, wherein the treatment guidance is for substance abuse and the ICD-10 code is F10.1 (alcohol abuse), F10.2 (alcohol dependence), F10.9 (alcohol use, unspecified), F11.1 (opioid abuse), F11.2 (opioid dependence), F11.9 (opioid use, unspecified), F12.1 (cannabis abuse), F12.2 (cannabis dependence), F12.9 (cannabis use, unspecified), F13.1 (sedative, hypnotic or anxiolytic-related abuse), F13.2 (sedative, hypnotic or anxiolytic-related dependence), F13.9 (sedative, hypnotic or anxiolytic-related use, unspecified), F14.1 (cocaine abuse), F14.2 (cocaine dependence), F14.9 (cocaine use, unspecified), F15.1 (other stimulant abuse), F15.2 (other stimulant dependence), F15.9 (other stimulant use, unspecified), F16.1 (hallucinogen abuse), F16.2 (hallucinogen dependence), F16.9 (hallucinogen use, unspecified), F17.2 (nicotine dependence), F18.1 (inhalant abuse), F18.2 (inhalant dependence), F18.9 (inhalant use, unspecified), F19.1 (other psychoactive substance abuse), F19.2 (other psychoactive substance dependence), F19.9 (other psychoactive substance use, unspecified), F55.0 (abuse of antacids), F55.1 (abuse of herbal or folk remedies), F55.2 (abuse of laxatives), F55.3 (abuse of steroids or hormones), F55.4 (abuse of vitamins), or F55.8 (abuse of other non-psychoactive substances).

392. A method for determining an allele status at a plurality of genomic loci in a subject, the method comprising:
a) amplifying, in a single in vitro reaction vessel, a first plurality of genomic loci, by polymerase chain reaction (PCR), from nucleic acids isolated from a sample obtained from the subject, in the presence of a plurality of fluorescently-labeled detection reagents, wherein the plurality of fluorescently-labeled detection reagents includes, for each respective genomic locus in the first plurality of genomic loci:
a first detection reagent that is specific for the presence of a first allele at the respective genomic locus, wherein the first allele is the most prevalent allele in a population of the species of the subject and the first detection reagent is labeled with a first fluorescent moiety, and
a second detection reagent that is specific for the presence of a second allele at the respective genomic locus, wherein the second allele is a minor allele in the population and the second detection reagent is labeled with a second fluorescent moiety that is distinguishable from the first fluorescent moiety;
b) during or after the amplifying a), detecting a fluorescent signal corresponding to the first fluorescent moiety and the second fluorescent moiety in the reaction vessel; and
c) when the contribution of the second fluorescent moiety to the fluorescent signal detected in the reaction vessel does not satisfy a threshold contribution, reporting that the subject does not carry the second allele at any of the first plurality of genomic loci.

393. The method of claim 392, wherein, when the contribution of the second fluorescent moiety to the fluorescent signal detected in the reaction vessel satisfies the threshold contribution, the method further comprises:
performing a first plurality of secondary allele detection assays, wherein each secondary allele detection assay in the first plurality of secondary allele detection assays determines the allele status at one respective genomic locus in the first plurality of genomic loci, and
reporting the allele status at each of the first plurality of genomic loci based on the first plurality of secondary allele detection assays.

394. A method for determining an allele status at a plurality of genomic loci in a subject, the method comprising:
a) amplifying, in a single in vitro reaction vessel, a first plurality of genomic loci, by polymerase chain reaction (PCR), from nucleic acids isolated from a sample obtained from the subject, in the presence of a plurality of fluorescently-labeled detection reagents, wherein the plurality of fluorescently-labeled detection reagents includes, for each respective genomic locus in the first plurality of genomic loci:
a first detection reagent that is specific for the presence of a first allele at the respective genomic locus, wherein the first allele is the most prevalent allele in a population of the species of the subject and the first detection reagent is labeled with a first fluorescent moiety, and
a second detection reagent that is specific for the presence of a second allele at the respective genomic locus, wherein the second allele is a minor allele in the population and the second detection reagent is labeled with a second fluorescent moiety that is distinguishable from the first fluorescent moiety;
b) during or after the amplifying a), detecting a fluorescent signal corresponding to the first fluorescent moiety and the second fluorescent moiety in the reaction vessel; and
c) when the contribution of the second fluorescent moiety to the fluorescent signal detected in the reaction vessel satisfies the threshold contribution:
performing a first plurality of secondary allele detection assays, wherein each secondary allele detection assay in the first plurality of secondary allele detection assays determines the allele status at one respective genomic locus in the first plurality of genomic loci, and
reporting the allele status at each of the first plurality of genomic loci based on the first plurality of secondary allele detection assays

395. The method of claim 394, wherein, when the contribution of the second fluorescent moiety to the fluorescent signal detected in the reaction vessel does not satisfy a threshold contribution, the method further comprises reporting that the subject does not carry the second allele at any of the first plurality of genomic loci.

396. A method for performing a high throughput genotyping assay, the method comprising:
a) dispensing, into each respective well in a first plurality of wells in a multiwell plate, in accordance with one or more template plate definitions associated with the high throughput genotyping assay, a respective template nucleic acid preparation, reagents for amplifying a first plurality of genomic loci, and a first plurality of fluorescently-labeled detection reagents, wherein:
the respective template nucleic acid preparation dispensed into each respective well is prepared from a respective biological sample obtained from a different test subject in a plurality of test subjects, and
the first plurality of fluorescently-labeled detection reagents includes, for each respective genomic locus in the first plurality of genomic loci:
a first detection reagent that is specific for the presence of a first allele at the respective genomic locus, wherein the first allele is the most prevalent allele in a population of the species of the subject and the first detection reagent is labeled with a first fluorescent moiety, and
a second detection reagent that is specific for the presence of a second allele at the respective genomic locus, wherein the second allele is a minor allele in the population and the second detection reagent is labeled with a second fluorescent moiety that is distinguishable from the first fluorescent moiety;
b) amplifying, after the dispensing a), the first plurality of genomic loci in each respective well;
c) detecting, during or after the amplifying b), in each respective well, a fluorescent signal corresponding to the first fluorescent moiety and the second fluorescent moiety in the reaction vessel; and
d) when the contribution of the second fluorescent moiety to the fluorescent signal detected in the respective well does not satisfy a threshold contribution, reporting that the corresponding subject in the plurality of subjects does not carry the second allele at any of the first plurality of genomic loci.

397. The method of claim 396, wherein, when the contribution of the second fluorescent moiety to the fluorescent signal detected in the respective well satisfies the threshold contribution, the method further comprises:
performing a first plurality of secondary allele detection assays using a template nucleic acid preparation from the corresponding subject in the plurality of subjects, wherein each secondary allele detection assay in the first plurality of secondary allele detection assays determines the allele status at one respective genomic locus in the first plurality of genomic loci, and
reporting the allele status of the corresponding subject at each of the first plurality of genomic loci based on the first plurality of secondary allele detection assays.

398. A method for performing a high throughput genotyping assay, the method comprising:
a) dispensing, into each respective well in a first plurality of wells in a multiwell plate, in accordance with one or more template plate definitions associated with the high throughput genotyping assay, a respective template nucleic acid preparation, reagents for amplifying a first plurality of genomic loci, and a first plurality of fluorescently-labeled detection reagents, wherein:
the respective template nucleic acid preparation dispensed into each respective well is prepared from a respective biological sample obtained from a different test subject in a plurality of test subjects, and
the first plurality of fluorescently-labeled detection reagents includes, for each respective genomic locus in the first plurality of genomic loci:
a first detection reagent that is specific for the presence of a first allele at the respective genomic locus, wherein the first allele is the most prevalent allele in a population of the species of the subject and the first detection reagent is labeled with a first fluorescent moiety, and
a second detection reagent that is specific for the presence of a second allele at the respective genomic locus, wherein the second allele is a minor allele in the population and the second detection reagent is labeled with a second fluorescent moiety that is distinguishable from the first fluorescent moiety;
b) amplifying, after the dispensing a), the first plurality of genomic loci in each respective well;
c) detecting, during or after the amplifying b), in each respective well, a fluorescent signal corresponding to the first fluorescent moiety and the second fluorescent moiety in the reaction vessel; and
d) when the contribution of the second fluorescent moiety to the fluorescent signal detected in the respective well satisfies the threshold contribution, the method further comprises:
performing a first plurality of secondary allele detection assays using a template nucleic acid preparation from the corresponding subject in the plurality of subjects, wherein each secondary allele detection assay in the first plurality of secondary allele detection assays determines the allele status at one respective genomic locus in the first plurality of genomic loci, and
reporting the allele status of the corresponding subject at each of the first plurality of genomic loci based on the first plurality of secondary allele detection assays.

399. The method of claim 398, wherein, when the contribution of the second fluorescent moiety to the fluorescent signal detected in the respective well does not satisfy a threshold contribution, the method further comprises reporting that the corresponding subject in the plurality of subjects does not carry the second allele at any of the first plurality of genomic loci.

400. A method for providing guidance for the treatment of a neuropsychiatric disorder in a subject, the method comprising:
a) determining the allele status for a plurality of genomic loci, wherein each respective loci in the plurality of loci is associated with a therapeutic efficacy of at least one therapy for a neuropsychiatric disorder, the determining comprising:
i) amplifying, in a first single in vitro reaction vessel, a first set of two or more genomic loci in the plurality of genomic loci, by polymerase chain reaction (PCR), from nucleic acids isolated from a sample obtained from the subject, in the presence of a plurality of fluorescently-labeled detection reagents, wherein the plurality of fluorescently-labeled detection reagents includes, for each respective genomic locus in the two or more genomic loci:
a first detection reagent that is specific for the presence of a first allele at the respective genomic locus, wherein the first allele is the most prevalent allele in a population of the species of the subject and the first detection reagent is labeled with a first fluorescent moiety, and
a second detection reagent that is specific for the presence of a second allele at the respective genomic locus, wherein the second allele is a minor allele in the population and the second detection reagent is labeled with a second fluorescent moiety that is distinguishable from the first fluorescent moiety;
ii) during or after the amplifying i), detecting a fluorescent signal corresponding to the first fluorescent moiety and the second fluorescent moiety in the first single reaction vessel; and
iii) when the contribution of the second fluorescent moiety to the fluorescent signal detected in the reaction vessel does not satisfy a threshold contribution, determining that the subject is homozygous for the first allele at each respective loci in the first set of two or more genomic loci;
b) associating the allele status determined for the plurality of genomic loci with one or more recommendations for the treatment of the neuropsychiatric disorder; and
c) generating a patient-specific report comprising the one or more recommendations for the treatment of the neuropsychiatric disorder.

401. The method of claim 400, wherein, when the contribution of the second fluorescent moiety to the fluorescent signal detected in the reaction vessel satisfies the threshold contribution, the method further comprises performing a first plurality of secondary allele detection assays, wherein each secondary allele detection assay in the first plurality of secondary allele detection assays determines the allele status at one respective genomic locus in the first set of two or more genomic loci, thereby determining the allele status at each respective loci in the first set of two or more genomic loci.

402. A method for providing guidance for the treatment of a neuropsychiatric disorder in a subject, the method comprising:
a) determining the allele status for a plurality of genomic loci, wherein each respective loci in the plurality of loci is associated with a therapeutic efficacy of at least one therapy for a neuropsychiatric disorder, the determining comprising:
i) amplifying, in a first single in vitro reaction vessel, a first set of two or more genomic loci in the plurality of genomic loci, by polymerase chain reaction (PCR), from nucleic acids isolated from a sample obtained from the subject, in the presence of a plurality of fluorescently-labeled detection reagents, wherein the plurality of fluorescently-labeled detection reagents includes, for each respective genomic locus in the two or more genomic loci:
a first detection reagent that is specific for the presence of a first allele at the respective genomic locus, wherein the first allele is the most prevalent allele in a population of the species of the subject and the first detection reagent is labeled with a first fluorescent moiety, and
a second detection reagent that is specific for the presence of a second allele at the respective genomic locus, wherein the second allele is a minor allele in the population and the second detection reagent is labeled with a second fluorescent moiety that is distinguishable from the first fluorescent moiety;
ii) during or after the amplifying i), detecting a fluorescent signal corresponding to the first fluorescent moiety and the second fluorescent moiety in the first single reaction vessel; and
iii) when the contribution of the second fluorescent moiety to the fluorescent signal detected in the reaction vessel satisfies the threshold contribution, performing a first plurality of secondary allele detection assays, wherein each secondary allele detection assay in the first plurality of secondary allele detection assays determines the allele status at one respective genomic locus in the first set of two or more genomic loci, thereby determining the allele status at each respective loci in the first set of two or more genomic loci;
b) associating the allele status determined for the plurality of genomic loci with one or more recommendations for the treatment of the neuropsychiatric disorder; and
c) generating a patient-specific report comprising the one or more recommendations for the treatment of the neuropsychiatric disorder.

403. The method of claim 402, wherein, when the contribution of the second fluorescent moiety to the fluorescent signal detected in the reaction vessel does not satisfy a threshold contribution, the method further comprises determining that the subject is homozygous for the first allele at each respective loci in the first set of two or more genomic loci, and

404. A method for providing treatment guidance in a subject, the method comprising:
a) determining the allele status for a plurality of genomic loci, wherein the determining comprises:
i) amplifying, in a first single in vitro reaction vessel, a first set of two or more genomic loci in the plurality of genomic loci, by polymerase chain reaction (PCR), from nucleic acids isolated from a sample obtained from the subject, in the presence of a plurality of fluorescently-labeled detection reagents, wherein the plurality of fluorescently-labeled detection reagents includes, for each respective genomic locus in the two or more genomic loci:
a first detection reagent that is specific for the presence of a first allele at the respective genomic locus, wherein the first allele is the most prevalent allele in a population of the species of the subject and the first detection reagent is labeled with a first fluorescent moiety, and
a second detection reagent that is specific for the presence of a second allele at the respective genomic locus, wherein the second allele is a minor allele in the population and the second detection reagent is labeled with a second fluorescent moiety that is distinguishable from the first fluorescent moiety;
ii) during or after the amplifying i), detecting a fluorescent signal corresponding to the first fluorescent moiety and the second fluorescent moiety in the first single reaction vessel; and
iii) when the contribution of the second fluorescent moiety to the fluorescent signal detected in the reaction vessel does not satisfy a threshold contribution, determining that the subject is homozygous for the first allele at each respective loci in the first set of two or more genomic loci;
b) associating the allele status determined for the plurality of genomic loci with one or more recommendations for the treatment of the neuropsychiatric disorder; and
c) generating a patient-specific report comprising the one or more recommendations for the treatment of a condition in fulfillment of an ICD-10 code selected from the group consisting of F31.0, F31.1, F31.2, F31.3, F31.5, F31.6, F31.7, F31.8, F31.9, F32.0, F32.2, F32.3, F32.4, F32.5, F32.8, F32.9, F33.0, F33.1, F33.2, F33.3, F33.4, F33.8, F33.9, F40.0, F40.1, F40.2, F40.8, F40.9, F41.0, F41.1, F41.3, F41.8, F41.9, F42.2, F42.3, F42.4, F42.8, F42.9, F60.5, F90.0, F90.1, F90.2, F90.8, F90.9, F43.1, F84.0, F20.0, F20.1, F20.2, F20.3, F20.5, F20.8, F20.9, F60.0, F60.1, F60.2, F60.3, F60.4, F60.5, F60.6, F60.7, F60.8, F60.9, F07.0, F07.8, F07.9, G89.2, G89.4, F10.1, F10.2, F10.9, F11.1, F11.2, F11.9, F12.1, F12.2, F12.9, F13.1, F13.2, F13.9, F14.1, F14.2, F14.9, F15.1, F15.2, F15.9, F16.1, F16.2, F16.9, F17.2, F18.1, F18.2, F18.9, F19.1, F19.2, F19.9, F55.0, F55.1, F55.2, F55.3, F55.4, and F55.8.

405. The method of claim 404, wherein, when the contribution of the second fluorescent moiety to the fluorescent signal detected in the reaction vessel satisfies the threshold contribution, the method further comprises performing a first plurality of secondary allele detection assays, wherein each secondary allele detection assay in the first plurality of secondary allele detection assays determines the allele status at one respective genomic locus in the first set of two or more genomic loci, thereby determining the allele status at each respective loci in the first set of two or more genomic loci

406. A method for providing treatment guidance in a subject, the method comprising:
a) determining the allele status for a plurality of genomic loci, wherein the determining comprises:
i) amplifying, in a first single in vitro reaction vessel, a first set of two or more genomic loci in the plurality of genomic loci, by polymerase chain reaction (PCR), from nucleic acids isolated from a sample obtained from the subject, in the presence of a plurality of fluorescently-labeled detection reagents, wherein the plurality of fluorescently-labeled detection reagents includes, for each respective genomic locus in the two or more genomic loci:
a first detection reagent that is specific for the presence of a first allele at the respective genomic locus, wherein the first allele is the most prevalent allele in a population of the species of the subject and the first detection reagent is labeled with a first fluorescent moiety, and
a second detection reagent that is specific for the presence of a second allele at the respective genomic locus, wherein the second allele is a minor allele in the population and the second detection reagent is labeled with a second fluorescent moiety that is distinguishable from the first fluorescent moiety;
ii) during or after the amplifying i), detecting a fluorescent signal corresponding to the first fluorescent moiety and the second fluorescent moiety in the first single reaction vessel; and
iii) when the contribution of the second fluorescent moiety to the fluorescent signal detected in the reaction vessel satisfies the threshold contribution, performing a first plurality of secondary allele detection assays, wherein each secondary allele detection assay in the first plurality of secondary allele detection assays determines the allele status at one respective genomic locus in the first set of two or more genomic loci, thereby determining the allele status at each respective loci in the first set of two or more genomic loci;
b) associating the allele status determined for the plurality of genomic loci with one or more recommendations for the treatment of the neuropsychiatric disorder; and
c) generating a patient-specific report comprising the one or more recommendations for the treatment of a condition in fulfillment of an ICD-10 code selected from the group consisting of F31.0, F31.1, F31.2, F31.3, F31.5, F31.6, F31.7, F31.8, F31.9, F32.0, F32.2, F32.3, F32.4, F32.5, F32.8, F32.9, F33.0, F33.1, F33.2, F33.3, F33.4, F33.8, F33.9, F40.0, F40.1, F40.2, F40.8, F40.9, F41.0, F41.1, F41.3, F41.8, F41.9, F42.2, F42.3, F42.4, F42.8, F42.9, F60.5, F90.0, F90.1, F90.2, F90.8, F90.9, F43.1, F84.0, F20.0, F20.1, F20.2, F20.3, F20.5, F20.8, F20.9, F60.0, F60.1, F60.2, F60.3, F60.4, F60.5, F60.6, F60.7, F60.8, F60.9, F07.0, F07.8, F07.9, G89.2, G89.4, F10.1, F10.2, F10.9, F11.1, F11.2, F11.9, F12.1, F12.2, F12.9, F13.1, F13.2, F13.9, F14.1, F14.2, F14.9, F15.1, F15.2, F15.9, F16.1, F16.2, F16.9, F17.2, F18.1, F18.2, F18.9, F19.1, F19.2, F19.9, F55.0, F55.1, F55.2, F55.3, F55.4, and F55.8.

407. The method of claim 406, wherein, when the contribution of the second fluorescent moiety to the fluorescent signal detected in the reaction vessel does not satisfy a threshold contribution, determining that the subject is homozygous for the first allele at each respective loci in the first set of two or more genomic loci, and


SEQUENCE LISTING (8 sequences)

SEQ ID NO: 1 - DNA, 61 bp, Artificial Sequence
  Synthetic genomic nucleotide and targeted loci
ggctgaacacgtccccgaagcggcgccgcaagctgcagagggagggtcagggcctcttgtc

SEQ ID NO: 2 - DNA, 52 bp, Artificial Sequence
  Synthetic genomic nucleotide and targeted loci
gattgaacgtgtgattggcagaaactcggagcccctgcatgcaagacaggag

SEQ ID NO: 3 - DNA, 52 bp, Artificial Sequence
  Synthetic genomic nucleotide and targeted loci
gatgggctcacgctgcacatccggagtgtaggatcatgagcaggaggcccca

SEQ ID NO: 4 - DNA, 52 bp, Artificial Sequence
  Synthetic genomic nucleotide and targeted loci
atggggaagaggagcattgaggaccagtgttcaagaggaagcccgctgcctt

SEQ ID NO: 5 - DNA, 52 bp, Artificial Sequence
  Synthetic genomic nucleotide and targeted loci
cggcgtttctccctcatgacgctgcaggaattttgggatggggaagaggagc

SEQ ID NO: 6 - DNA, 52 bp, Artificial Sequence
  Synthetic genomic nucleotide and targeted loci
gcagtgaaggaagccctgattgatccttggagaggagttttctggaagaggc

SEQ ID NO: 7 - DNA, 52 bp, Artificial Sequence
  Synthetic genomic nucleotide and targeted loci
tccacatgcagcaggttgcccagccctgggcagtggcagggggcctggtggg

SEQ ID NO: 8 - DNA, 52 bp, Artificial Sequence
  Synthetic genomic nucleotide and targeted loci
cctatgtttgttattttcaggaaaactggatttgtgtgggagagggcctggc